(19) United States
(12) Patent Application Publication
(10) Pub. No.: US 2022/0296628 A1
(43) Pub. Date: Sep. 22, 2022

(54) RNA COMBINATIONS AND COMPOSITIONS WITH DECREASED IMMUNOSTIMULATORY PROPERTIES

(71) Applicant: CureVac AG, Tübingen (DE)

(72) Inventors: Andreas THESS, Tübingen (DE); Frédéric CHEVESSIER-TÜNNESEN, Tübingen (DE); Johannes LUTZ, Tübingen (DE); Thomas SCHLAKE, Tübingen (DE)

(73) Assignee: CureVac AG, Tübingen (DE)

(21) Appl. No.: 17/634,958

(22) PCT Filed: Aug. 11, 2020
(86) PCT No.: PCT/EP2020/072516

§ 371 Date: Feb. 11, 2022

(30) Foreign Application Priority Data
Aug. 14, 2019 (EP) PCT/EP2019/071878

(51) Int. Cl. A61K 31/713; C12N 15/117; A61P 37/04

(52) U.S. Cl. CPC A61K 31/713; C12N 15/117; A61P 37/04; C12N 2310/17; C12N 2320/53; C12N 2310/321; C12N 2310/3513

(57) ABSTRACT

The invention relates inter alia to a combination comprising (i) a first component comprising at least one therapeutic RNA and (ii) a second component comprising at least one antagonist of at least one RNA sensing pattern recognition receptor. Further provided are compositions comprising at least one therapeutic RNA and at least one antagonist of at least one RNA sensing pattern recognition receptor. The combination of the two components is able to reduce immunostimulatory properties of the first component as well as promote expression after administration. Additionally, first and second medical uses, and methods of treating or preventing diseases, disorders or conditions are provided.

### INTRODUCTION

The invention relates inter alia to a combination comprising (i) a first component comprising at least one therapeutic RNA and (ii) a second component comprising at least one antagonist of at least one RNA sensing pattern recognition receptor. Further provided are compositions comprising at least one therapeutic RNA and at least one antagonist of at least one RNA sensing pattern recognition receptor. Additionally, first and second medical uses, and methods of treating or preventing diseases, disorders or conditions are provided.

RNA-based therapeutics can be used in e.g. passive and active immunotherapy, protein replacement therapy, or genetic engineering. Accordingly, therapeutic RNA has the potential to provide highly specific and individual treatment options for the therapy of a large variety of diseases, disorders, or conditions.

Besides used as vaccines, RNA molecules may also be used as therapeutics for replacement therapies, such as e.g. protein replacement therapies for substituting missing or mutated proteins such as growth factors or enzymes, in patients. However, successful development of safe and efficacious RNA-based replacement therapies are based on different preconditions compared to vaccines. When applying coding RNA for protein replacement therapies, the therapeutic coding RNA should confer sufficient expression of the protein of interest in terms of expression level and duration and minimal stimulation of the innate immune system to avoid inflammation in the patient to be treated, and to avoid specific immune responses against the administered RNA molecule and the encoded protein.

Whereas the inherent immunostimulatory property of therapeutic RNA may be considered as a desirable feature for vaccines, this effect may cause undesired complications in replacement therapies. This is especially the case for the treatment of chronic diseases in which the RNA therapeutic needs to be administered repeatedly over an extended period of time. The potential capacity of therapeutic RNA to elicit innate immune responses may represent limitations to its in vivo application.

Induction and/or enhancement of immune responses of the innate and/or the adaptive immune system plays an important role in numerous diseases. Some innate immune receptors have been identified that are specialized to detect foreign or damage-associated nucleic acids. One of these groups of nucleic acid-sensing immune receptors are the Toll-like receptors (TLRs) which are pattern recognition receptors (PRR) that are preferentially located in the endolysosomal compartment of distinct immune cell subsets and certain somatic cells. The latter receptors serve to identify pathogen-associated molecular patterns (PAMPs) and danger-associated molecular patterns (DAMPs). The PPRs act as the primary defense against pathogenic entities and control the activation and progression of the adaptive immunity by activating the production not only of pro-inflammatory cytokines, chemokines and interferons, but also B and T cells. Among the PPRs, the Toll-like Receptors (TLRs) are of special interest. Their discovery more than 30 years ago has improved knowledge in the regulation of innate immunity, inflammation and cytokines induction Stimulation of nucleic acid-sensing receptors typically results in the induction of cytokines (e.g., type I interferons) and chemokines to alarm neighboring cells, and e.g. to recruit immune cells. For example, TLR3, TLR7, TLR8 and TLR9 are intracellular TLRs that recognize nucleic acids (e.g. RNA) that are taken up by the cell via endocytosis and transferred to endosomes. Further nucleic-acid sensing immune receptors include RIG-1 family of helicases (e.g., RIG-I, MDA5, LGP2), NOD-like receptors, PKR, OAS, SAMHD1, ADAR1, IFIT1 and/or IFIT5.

Accordingly, the induction of innate immune responses, primarily mediated by RNA sensing pattern recognition receptors such as toll-like receptors 7 and 8, can compromise the effectiveness of RNA-based therapeutics and may therefore lead to reduced therapeutic efficacy. Even if the induction of a certain cytokine profile may be advantageous for prophylactic vaccines, a reactogenicity to the RNA vaccine characterized by e.g. fever and illness has to be avoided. Therefore it is a challenge in the field to find a balance between inducing an innate immune response to support an adaptive immune response while avoiding fever and illness.

In the art, that problem has been partially addressed by using modified RNA nucleotides. By introducing modified nucleotides, the therapeutic RNA can show reduced innate immune stimulation in vivo. However, therapeutic RNA comprising modified nucleotides often shows reduced expression or reduced activity in vivo because modifications can also prevent recruitment of beneficial RNA-binding proteins and thus impede activity of the therapeutic RNA, e.g. protein translation.

Prior art describes the use of immune regulatory oligonucleotide (IRO) with modified CpG motifs as antagonists of TLRs to inhibit and/or suppress a TLR-mediated immune response induced by endogenous and/or exogenous nucleic acids such as modified messenger RNA (mmRNA) therapeutics or DNA used in gene therapy (WO2017136399). Small synthetic oligodeoxynucleotides (ODN) containing unmethylated deoxycytidine-deoxyguanosine (CpG) dinucleotides are able to mimic the immune stimulatory activity of bacterial DNA via recognition by TLR9 (Pohar et al, Selectivity of Human TLR9 for Double CpG Motifs and Implications for the Recognition of Genomic DNA, J Immunol Mar. 1, 2017, 198 (5) 2093-2104 and El-Zayat et al Toll-like receptors activation, signaling, and targeting: an overview, Bulletin of the National Research Centre (2019) 43:187).

Summarizing the above, it is problematic to reduce immunostimulatory properties of a therapeutic RNA and, at the same time, to retain the efficacy, e.g. translatability of such an RNA in a cell and/or inducing an adaptive immune response. However, in most therapeutic settings, both features (reduced or low immunostimulatory properties, high translation rates in vivo) are of paramount importance for an RNA medicament.

The objects outlined above are solved by the claimed subject matter of the invention.

### Definitions

For the sake of clarity and readability the following definitions are provided. Any technical feature mentioned for these definitions may be read on each and every embodiment of the invention.

Percentages in the context of numbers should be understood as relative to the total number of the respective items. In other cases, and depending on the context, percentages should be understood as percentages by weight (wt.-%).

About: The term “about” is used when parameters or values do not necessarily need to be identical, i.e. 100% the same. Accordingly, “about” means, that a parameter or values may diverge by 0.1% to 20%, preferably by 0.1% to 10%; in particular, by 0.5%, 1%, 2%, 3%, 4%, 5%, 6%, 7%, 8%, 9%, 10%, 11%, 12%, 13%, 14%, 15%, 16%, 17%, 18%, 19%, 20%. The skilled person will know that e.g. certain parameters or values may slightly vary based on the method how the parameter was determined. For example, if a certain parameter or value is defined herein to have e.g. a length of “about 1000 nucleotides”, the length may diverge by 0.1% to 20%, preferably by 0.1% to 10%; in particular, by 0.5%, 1%, 2%, 3%, 4%, 5%, 6%, 7%, 8%, 9%, 10%, 11%, 12%, 13%, 14%, 15%, 16%, 17%, 18%, 19%, 20%. Accordingly, the skilled person will know that in that specific example, the length may diverge by 1 to 200 nucleotides, preferably by 1 to 100 nucleotides; in particular, by 5, 10, 20, 30, 40, 50, 60, 70, 80, 90, 100, 110, 120, 130, 140, 150, 160, 170, 180, 190, 200 nucleotides.

Adaptive immune response: The term “adaptive immune response” as used herein will be recognized and understood by the person of ordinary skill in the art, and is e.g. intended to refer to an antigen-specific response of the immune system (the adaptive immune system). Antigen specificity allows for the generation of responses that are tailored to specific pathogens or pathogen-infected cells. The ability to mount these tailored responses is usually maintained in the body by “memory cells” (B-cells). In the context of the invention, an antigen may be provided by the at least one therapeutic RNA of the inventive combination/composition.

Antibody, antibody fragment: As used herein, the term “antibody” includes both an intact antibody and an antibody fragment. Typically, an intact “antibody” is an immunoglobulin that specifically binds to a particular antigen. An antibody may be a member of any immunoglobulin class, including any of the human classes: IgG, IgM, IgE, IgA and IgD. Typically, an intact antibody is a tetramer. Each tetramer consists of two identical pairs of polypeptide chains, each pair having a “light” chain and a “heavy” chain. An “antibody fragment” includes a portion of an intact antibody, such as the antigen-binding or variable region of an antibody. Examples of antibody fragments include Fab, Fab′, F(ab′) 2 and Fv fragments; the tribes; Tetra; linear antibodies; single-chain antibody molecules; and multi specific antibodies formed from antibody fragments. E.g., the antibody fragments comprise isolated fragments, “Fv” fragments consisting of heavy and light chain variable regions, recombinant single chain polypeptide molecules in which the light and heavy chain variable regions are linked together by a peptide linker (“ScFv Proteins”) and minimal recognition units consisting of amino acid residues that mimic the hypervariable region. Examples of antigen-binding fragments of an antibody include, but are not limited to, Fab fragment, Fab′ fragment, F (ab′) 2 fragment, scFv fragment, Fv fragment, dsFv diabody, dAb fragment, fragment Fd′, Fd fragment and an isolated complementarity determining region (CDR). Suitable antibodies that may be encoded by the therapeutic RNA of the invention include monoclonal antibodies, polyclonal antibodies, antibody mixtures or cocktails, human or humanized antibodies, chimeric antibodies, Fab fragments, or bispecific antibodies. In the context of the invention, an antibody may be provided by the at least one therapeutic RNA of the inventive combination/composition.

Agonist: the term “agonist” is used for a substance that binds to a receptor of a cell and induces a response. An agonist often mimics the action of a naturally occurring substance such as a ligand.

Antagonist: The “term antagonist” generally refers to a substance that attenuates the effect of an agonist Antigen: The term “antigen” as used herein will be recognized and understood by the person of ordinary skill in the art, and is e.g. intended to refer to a substance which may be recognized by the immune system, preferably by the adaptive immune system, and is capable of triggering an antigen-specific immune response, e.g. by formation of antibodies and/or antigen-specific T cells as part of an adaptive immune response. Typically, an antigen may be or may comprise a peptide or protein which may be presented by the MHC to T-cells. Also fragments, variants and derivatives of peptides or proteins derived from e.g. cancer antigens comprising at least one epitope may be understood as antigens. In the context of the present invention, an antigen may be the product of translation of a provided therapeutic RNA (e.g. coding RNA, replicon RNA, mRNA). The term “antigenic peptide or protein” will be recognized and understood by the person of ordinary skill in the art, and is e.g. intended to refer to a peptide or protein derived from a (antigenic) protein which may stimulate the body's adaptive immune system to provide an adaptive immune response. Therefore an “antigenic peptide or protein” comprises at least one epitope or antigen of the protein it is derived from (e.g. a tumor antigen, a viral antigen, a bacterial antigen, a protozoan antigen). In the context of the invention, an antigen may be provided by the at least one therapeutic RNA of the inventive combination/composition.

Carrier: The term “carrier” encompasses any excipient, diluent, filler, salt, buffer, stabilizer, solubilizer, oil, lipid, lipid containing vesicle, microspheres, liposomal encapsulation, or other material well known in the art for use in pharmaceutical formulations. It will be understood that the characteristics of the carrier, excipient, or diluent will depend on the route of administration for a particular application. The preparation of pharmaceutically acceptable formulations containing these materials is described in, e. g, Remington's Pharmaceutical Sciences, 18th Edition, ed. A. Gennaro, Mack Publishing Co., Easton, Pa., 1990 Cationic, cationisable: Unless a different meaning is clear from the specific context, the term “cationic” means that the respective structure bears a positive charge, either permanently or not permanently but in response to certain conditions such as e.g. pH. Thus, the term “cationic” covers both “permanently cationic” and “cationisable”. The term “cationisable” as used herein means that a compound, or group or atom, is positively charged at a lower pH and uncharged at a higher pH of its environment. Also in non-aqueous environments where no pH value can be determined, a cationisable compound, group or atom is positively charged at a high hydrogen ion concentration and uncharged at a low concentration or activity of hydrogen ions. It depends on the individual properties of the cationisable or polycationisable compound, in particular the pKa of the respective cationisable group or atom, at which pH or hydrogen ion concentration it is charged or uncharged. In diluted aqueous environments, the fraction of cationisable compounds, groups or atoms bearing a positive charge may be estimated using the so-called Henderson-Hasselbalch equation which is well-known to a person skilled in the art. E.g., if a compound or moiety is cationisable, it is preferred that it is positively charged at a pH value of about 1 to 9, preferably 4 to 9, 5 to 8 or even 6 to 8, more preferably of a pH value of or below 9, of or below 8, of or below 7, most preferably at physiological pH values, e.g. about 7.3 to 7.4, i.e. under physiological conditions, particularly under physiological salt conditions of the cell in vivo. In embodiments, it is preferred that the cationisable compound or moiety is predominantly neutral at physiological pH values, e.g. about 7.0-7.4, but becomes positively charged at lower pH values. In some embodiments, the preferred range of pKa for the cationisable compound or moiety is about 5 to about 7.

Derived from: The term “derived from” as used throughout the present specification in the context of a nucleic acid, i.e. for a nucleic acid “derived from” (another) nucleic acid, means that the nucleic acid, which is derived from (another) nucleic acid, shares e.g. at least about 70%, 80, 90%, 91%, 92%, 93%, 94%, 95%, 96%, 97%, 98%, or about 99% sequence identity with the nucleic acid from which it is derived. The skilled person is aware that sequence identity is typically calculated for the same types of nucleic acids, i.e. for DNA sequences or for RNA sequences. Thus, it is understood, if a DNA is “derived from” an RNA or if an RNA is “derived from” a DNA, in a first step the RNA sequence is converted into the corresponding DNA sequence (in particular by replacing U by T throughout the sequence) or, vice versa, the DNA sequence is converted into the corresponding RNA sequence (in particular by replacing the T by U throughout the sequence). Thereafter, the sequence identity of the DNA sequences or the sequence identity of the RNA sequences is determined. Preferably, a nucleic acid “derived from” a nucleic acid also refers to nucleic acid, which is modified in comparison to the nucleic acid from which it is derived, e.g. in order to increase RNA stability even further and/or to prolong and/or increase protein production. In the context of amino acid sequences, the term “derived from” means that the amino acid sequence, which is derived from (another) amino acid sequence, shares e.g. at least about 70%, 80, 90%, 91%, 92%, 93%, 94%, 95%, 96%, 97%, 98%, or about 99% sequence identity with the amino acid sequence from which it is derived.

CRISPR-associated protein: The term “CRISPR-associated protein” or “CRISPR-associated endonuclease” will be recognized and understood by the person of ordinary skill in the art. The term “CRISPR-associated protein” refers to RNA-guided endonucleases that are part of a CRISPR (Clustered Regularly Interspaced Short Palindromic Repeats) system (and their homologs, variants, fragments or derivatives), which is used by prokaryotes to confer adaptive immunity against foreign DNA elements. CRISPR-associated proteins include, without limitation, Cas9, Cpf1 (Cas12), C2c1, C2c3, C2c2, Cas13, CasX and CasY. As used herein, the term “CRISPR-associated protein” includes wild-type proteins as well as homologs, variants, fragments and derivatives thereof. Therefore, when referring to artificial nucleic acid molecules encoding Cas9, Cpf1 (Cas12), C2c1, C2c3, and C2c2, Cas13, CasX and CasY, said artificial nucleic acid molecules may encode the respective wild-type proteins, or homologs, variants, fragments and derivatives thereof. Besides Cas9 and Cas12 (Cpf1), several other CRISPR-associated protein exist that are suitable for genetic engineering in the context of the invention, including Cas13, CasX and CasY. In the context of the invention, a CRISPR-associated protein may be provided by the at least one therapeutic RNA of the inventive combination or composition.

Fragment: The term “fragment” as used throughout the present specification in the context of a nucleic acid sequence or an amino acid (aa) sequence may typically be a shorter portion of a full-length sequence of e.g. a nucleic acid sequence or an amino acid sequence. A fragment typically consists of a sequence that is identical to the corresponding stretch within the full-length sequence. The term “fragment” as used throughout the present specification in the context of proteins or peptides may, typically, comprise a sequence of a protein or peptide as defined herein, which is, with regard to its amino acid sequence (or its encoded nucleic acid molecule), N-terminally and/or C-terminally truncated compared to the amino acid sequence of the original (native) protein (or its encoded nucleic acid molecule). Such truncation may thus occur either on the aa level or correspondingly on the nucleic acid level. A sequence identity with respect to such a fragment as defined herein may therefore preferably refer to the entire protein or peptide as defined herein or to the entire (coding) nucleic acid molecule of such a protein or peptide. Fragments of antigenic proteins or peptides may comprise at least one epitope of those proteins or peptides. Furthermore also domains of a protein, like the extracellular domain, the intracellular domain or the transmembrane domain and shortened or truncated versions of a protein may be understood to comprise a fragment of a protein.

Heterologous: The terms “heterologous” or “heterologous sequence” as used throughout the present specification in the context of a nucleic acid sequence or an amino acid sequence refers to a sequence (e.g. DNA, RNA, amino acid) will be recognized and understood by the person of ordinary skill in the art, and is intended to refer to a sequence that is derived from another gene, from another allele, from another species. Two sequences are typically understood to be “heterologous” if they are not derivable from the same gene or in the same allele. I.e., although heterologous sequences may be derivable from the same organism, they naturally (in nature) do not occur in the same nucleic acid molecule, such as e.g. in the same RNA or protein.

Identity (of a sequence): The term “identity” as used throughout the present specification in the context of a nucleic acid sequence or an amino acid sequence will be recognized and understood by the person of ordinary skill in the art, and is e.g. intended to refer to the percentage to which two sequences are identical. To determine the percentage to which two sequences are identical, e.g. nucleic acid sequences or amino acid (aa) sequences as defined herein, preferably the aa sequences encoded by the nucleic acid sequence as defined herein or the aa sequences themselves, the sequences can be aligned in order to be subsequently compared to one another. Therefore, e.g. a position of a first sequence may be compared with the corresponding position of the second sequence. If a position in the first sequence is occupied by the same residue as is the case at a position in the second sequence, the two sequences are identical at this position. If this is not the case, the sequences differ at this position. If insertions occur in the second sequence in comparison to the first sequence, gaps can be inserted into the first sequence to allow a further alignment. If deletions occur in the second sequence in comparison to the first sequence, gaps can be inserted into the second sequence to allow a further alignment. The percentage to which two sequences are identical is then a function of the number of identical positions divided by the total number of positions including those positions which are only occupied in one sequence. The percentage to which two sequences are identical can be determined using an algorithm, e.g. an algorithm integrated in the BLAST program.

Immune response: The term “immune response” will be recognized and understood by the person of ordinary skill in the art, and is e.g. intended to refer to a specific reaction of the adaptive immune system to a particular antigen (so called specific or adaptive immune response) or an unspecific reaction of the innate immune system (so called unspecific or innate immune response), or a combination thereof.

Immune system: The term “immune system” will be recognized and understood by the person of ordinary skill in the art, and is e.g. intended to refer to a system of the organism that may protect the organisms from infection. If a pathogen succeeds in passing a physical barrier of an organism and enters this organism, the innate immune system provides an immediate, but non-specific response. If pathogens evade this innate response, vertebrates possess a second layer of protection, the adaptive immune system. Here, the immune system adapts its response during an infection to improve its recognition of the pathogen. This improved response is then retained after the pathogen has been eliminated, in the form of an immunological memory, and allows the adaptive immune system to mount faster and stronger attacks each time this pathogen is encountered. According to this, the immune system comprises the innate and the adaptive immune system. Each of these two parts typically contains so called humoral and cellular components.

Treatment: The term “treatment” generally refers to an approach intended to obtain a beneficial or desired results, Which may include alleviation of symptoms, or delaying or ameliorating a disease progression.

Messenger RNA (mRNA): The term “messenger RNA” (mRNA) refers to one type of RNA molecule. In vivo, transcription of DNA usually results in the so-called premature RNA which has to be processed into so-called messenger RNA, usually abbreviated as mRNA. Typically, an mRNA comprises a 5-cap, a 5′-UTR, an open reading frame/coding sequence, a 3-UTR and a poly(A).

Nucleoside: The term “nucleoside” generally refers to compounds consisting of a sugar, usually ribose or deoxyribose, and a purine or pyrimidine base.

Nucleotide: The term “nucleotide” generally refers to a nucleoside comprising a phosphate group attached to the sugar.

Nucleic acid sequence, RNA sequence: The terms “nucleic acid sequence” or “RNA sequence” will be recognized and understood by the person of ordinary skill in the art, and are e.g. intended to refer to particular and individual order of the succession of its nucleotides or amino acids respectively.

Variant (of a sequence): The term “variant” as used throughout the present specification in the context of a nucleic acid sequence will be recognized and understood by the person of ordinary skill in the art, and is e.g. intended to refer to a variant of a nucleic acid sequence derived from another nucleic acid sequence. E.g., a variant of a nucleic acid sequence may exhibit one or more nucleotide deletions, insertions, additions and/or substitutions compared to the nucleic acid sequence from which the variant is derived. A variant of a nucleic acid sequence may at least 50%, 60%, 70%, 80%, 90%, or 95% identical to the nucleic acid sequence the variant is derived from. The variant is preferably a functional variant in the sense that the variant has retained at least 50%, 60%, 70%, 80%, 90%, or 95% or more of the function of the sequence where it is derived from. A “variant” of a nucleic acid sequence may have at least 70%, 75%, 80%, 85%, 90%, 95%, 98% or 99% nucleotide identity over a stretch of at least 10, 20, 30, 50, 75 or 100 nucleotide of such nucleic acid sequence.

The term “variant” as used throughout the present specification in the context of proteins or peptides will be recognized and understood by the person of ordinary skill in the art, and is e.g. intended to refer to a proteins or peptide variant having an amino acid sequence which differs from the original sequence in one or more mutation(s), such as one or more substituted, inserted and/or deleted amino acid(s). Preferably, these fragments and/or variants have the same biological function or specific activity compared to the full-length native protein, e.g. its specific antigenic property. “Variants” of proteins or peptides as defined herein may comprise conservative amino acid substitution(s) compared to their native, i.e. non-mutated physiological, sequence. A “variant” of a protein or peptide may have at least 70%, 75%, 80%, 85%, 90%, 95%, 98% or 99% amino acid identity over a stretch of at least 10, 20, 30, 50, 75 or 100 amino acids of such protein or peptide. Preferably, a variant of a protein comprises a functional variant of the protein, which means that the variant exerts the same effect or functionality or at least 40%, 50%, 60%, 70%, 80%, 90%, or 95% of the effect or functionality as the protein it is derived from.

### SHORT DESCRIPTION OF THE INVENTION

The present invention is based on the finding that the co-administration of a component comprising at least one antagonist of at least one RNA sensing pattern recognition receptor results in a reduced (innate) immune stimulation induced by a therapeutic RNA for example as compared to administration of the corresponding therapeutic RNA alone. Surprisingly, co-administration of a component comprising at least one antagonist of at least one RNA sensing pattern recognition receptor preferably increases and/or prolongs the expression of a peptide or protein encoded by the therapeutic RNA.

As outlined in the Example section, the inventors found that the addition of a chemically modified oligonucleotide had an immunosuppressive effect to a co-administered immune stimulatory RNA sequence (“RNAdjuvant”) (see e.g. FIG. 1A). Additionally, the inventors showed that a chemically modified oligonucleotide efficiently antagonised the immunostimulation of RNA (see e.g. Example 2 (in vitro) or Example 3 (in vivo)), an unwanted side-effect that is typically triggered by RNA sensing receptors. The oligonucleotide used herein has been described to antagonize Toll-like receptors (TLR) 7 and 8, RNA sensing pattern recognition receptors involved in innate immune responses (see Schmitt et al. 2017. RNA 23:1344-135). The invention is based on the findings showing that a combination or composition comprising at least one antagonist of at least one RNA sensing receptor and at least one therapeutic RNA can reduce the immunostimulatory properties of said at least one therapeutic RNA. Unexpectedly, the addition of the antagonistic oligonucleotide also increased and/or prolonged expression of the encoded protein of the co-administered therapeutic RNA, suggesting that a combination or composition comprising an antagonist of at least one RNA sensing pattern recognition receptor (e.g. a TLR7 antagonist) and therapeutic RNA (e.g. mRNA) results in reduced immunostimulation and increased and/or prolonged protein expression—features that are of paramount importance for most RNA-based medicaments.

In a first aspect, the present invention relates to a combination comprising (i) at least one first component comprising at least one therapeutic RNA and (ii) at least one second component comprising at least one antagonist of at least one RNA sensing pattern recognition receptor.

In a second aspect, the present invention relates to a pharmaceutical composition comprising or consisting of a combination comprising (i) at least one therapeutic RNA, preferably as described in the first aspect; (ii) at least one antagonist of at least one RNA sensing pattern recognition receptor, preferably as described in the first aspect, and optionally at least one pharmaceutically acceptable carrier.

In a third aspect, the present invention relates to a kit or kit of parts comprising the first and the second component of the combination of the first aspect, and/or comprising the composition of the second aspect.

In a fourth aspect, the invention relates to the combination of the first aspect, the composition of the second aspect, or the kit or kit of parts of the third aspect for use as a medicament.

In further aspects, the invention relates to the combination of the first aspect, the composition of the second aspect, or the kit or kit of parts of the third aspect for use as a medicament in a chronic medical treatment or as a vaccine. Other aspects relate methods of treating or preventing a disease, disorder, or condition, a method of reducing the (innate) immune stimulation of a therapeutic RNA, a method of reducing the reactogenicity of a therapeutic RNA composition, and a method of increasing and/or prolonging the expression of a peptide or protein encoded by a (coding) therapeutic RNA.

### DETAILED DESCRIPTION OF THE INVENTION

The present application is filed together with a sequence listing in electronic format, which is part of the description of the present application (WIPO standard ST.25). The information contained in the electronic format of the sequence listing filed together with this application is incorporated herein by reference in its entirety. For many sequences, the sequence listing also provides additional detailed information, e.g. regarding certain structural features, sequence modifications, GenBank identifiers, or additional detailed information. In particular, such information is provided under numeric identifier <223> in the WIPO standard ST.25 sequence listing. Accordingly, information provided under said numeric identifier <223> is explicitly included herein in its entirety and has to be understood as integral part of the description of the underlying invention.

Combination

In a first aspect, the invention is inter alia directed to a combination comprising a first component comprising a therapeutic RNA and a second component comprising an antagonist of an RNA sensing pattern recognition receptor.

In the context of the present invention, the term “combination” preferably means a combined occurrence of the at least one therapeutic RNA (herein referred to as “first component”) and of the at least one antagonist of at least one RNA sensing pattern recognition receptor (herein referred to as “second component”). Therefore, said combination may occur either as one composition, comprising all these components in one and the same composition or mixture (but as separate entities), or may occur as a kit of parts, wherein the different components form different parts of such a kit of parts (as defined in the third aspect). Thus, the administration of the first and the second component of the combination may occur either simultaneously or timely staggered, either at the same site of administration or at different sites of administration, as further outlined below. The components may be formulated together as a co-formulation (as further described in the context of the second aspect), or may be formulated as different separate formulations (and optionally combined after formulation) as outlined below.

In the first aspect, the combination comprises

(i) at least one first component comprising at least one therapeutic RNA;

(ii) at least one second component comprising at least one antagonist of at least one RNA sensing pattern recognition receptor.

In the following, advantageous embodiments and features of the at least one antagonist of at least one RNA sensing pattern recognition receptor of the second component are described. Notably, all described embodiments and features of said at least one antagonist described in the context of the inventive combination (first aspect) are likewise be applicable to the at least one antagonist of the pharmaceutical composition (second aspect), or the kit or kit of parts (third aspect), or to any further aspect described herein (e.g. medical use, method of treatment).

The term “Pattern recognition receptor” (PRR) as used throughout the present specification will be recognized and understood by the person of ordinary skill in the art, and is e.g. intended to refer to receptors that are part of the innate immune system. Germline-encoded PRRs are responsible for sensing the presence of microbe-specific molecules (such as bacterial or viral DNA or RNA) via recognition of conserved structures, which are called pathogen-associated molecular patterns (PAMPs). Recent evidence indicates that PRRs are also responsible for recognizing endogenous molecules released from damaged cells, termed damage-associated molecular patterns (DAMPs). Currently, four different classes of PRR families have been identified. These families include transmembrane proteins such as the Toll-like receptors (TLRs) and C-type lectin receptors (CLRs), as well as cytoplasmic proteins such as the Retinoic acid-inducible gene (RIG)-l-like receptors (RLRs) and NOD-like receptors (NLRs). Based on their localization, PRRs may be divided into membrane-bound PRRs and cytoplasmic PRRs and are expressed not only in macrophages and DCs but also in various nonprofessional immune cells. (Takeuchi and Akira 2010. Pattern Recognition Receptors and Inflammation, Cell, Volume 140, ISSUE 6, P805-820) Typical Pattern recognition receptor” (PRR) in the context of the invention are Toll-like receptors, NOD-like receptors, RIG-1 like receptors, PKR, OAS1, IFIT1 and IFIT5.

The term “innate immune system”, also known as non-specific (or unspecific) immune system, as used throughout the present specification will be recognized and understood by the person of ordinary skill in the art, and is e.g. intended to refer to a system that typically comprises the cells and mechanisms that defend the host from infection by other organisms in a non-specific manner. This means that the cells of the innate system may recognize and respond to pathogens in a generic way, but unlike the adaptive immune system, it does not confer long-lasting or protective immunity to the host. The innate immune system may be, e.g., activated by ligands (e.g. PAMPs) of “Pattern recognition receptors” (PRR) or other auxiliary substances such as lipopolysaccharides, TNF-alpha, CD40 ligand, or cytokines, monokines, lymphokines, interleukins or chemokines, IL-1, IL-2, IL-3, IL-4, IL-5, IL-6, IL-7, IL-8, IL-9, IL-10, IL-11, IL-12, IL-13, IL-14, IL-15, IL-16, IL-17, IL-18, IL-19, IL-20, IL-21, IL-22, IL-23, IL-24, IL-25, IL-26, IL-27, IL-28, IL-29, IL-30, IL-31, IL-32, IL-33, IFN-alpha, IFN-beta, IFN-gamma, GM-CSF, G-CSF, M-CSF, LT-beta, TNF-alpha, growth factors, and hGH, a ligand of human Toll-like receptor TLR1, TLR2, TLR3, TLR4, TLR5, TLR6, TLR7, TLR8, TLR9, TLR10, a ligand of murine Toll-like receptor TLR1, TLR2, TLR3, TLR4, TLR5, TLR6, TLR7, TLR8, TLR9, TLR10, TLR11, TLR12 or TLR13, a ligand of a NOD-like receptor, a ligand of a RIG-1 like receptor, an immunostimulatory nucleic acid, an immunostimulatory RNA (isRNA), a CpG-DNA, an antibacterial agent, an anti-viral agent, a ligand of PKR and OAS1 (e.g. long double stranded RNA) or a ligand of IFIT1 and IFIT5 (5′ppp RNA).

Typically, a response of the innate immune system (after e.g. sensing an RNA) includes recruiting immune cells to sites of infection, through the production of chemical factors, including specialized chemical mediators, called cytokines; activation of the complement cascade; identification and removal of foreign substances present in organs, tissues, the blood and lymph, by specialized white blood cells; activation of the adaptive immune system; and/or acting as a physical and chemical barrier to infectious agents. Typically, protein synthesis is also reduced during the innate immune response. The inflammatory response is orchestrated by proinflammatory cytokines such as tumor necrosis factor (TNF), interleukin (IL)-1, and IL-6. These cytokines are pleiotropic proteins that regulate the cell death of inflammatory tissues, modify vascular endothelial permeability, recruit blood cells to inflamed tissues, and induce the production of acute-phase proteins PRRs can be activated by a broad variety of pathogen associated molecular patterns (PAMPs) for example PAMPs derived from viruses, bacteria, fungi, protozoa, ranging from lipoproteins, carbohydrates, lipopolysaccharides, and various types of nucleic acids (DNA, RNA, dsRNA, non-capped RNA or 5′ ppp RNA).

PPRs may be present in different compartments of a cell (e.g. located in the membrane of an endosome or located in the cytoplasm). Upon sensing PAMPs, the PRRs trigger signaling cascades leading inter alia to expression of e.g. cytokines, chemokines. For example, toll like receptor 3 (TLR-3) typically detects long double-stranded RNA (>40 bp) and is also expressed on the surface of certain cell types. The expression of TLR7 in the human immune system is typically restricted to B cells and PDC, TLR8 is preferentially expressed in myeloid immune cells. Consequently, TLR7 ligands drive B cell activation and the production of large amounts of IFN-alpha in Plasmacytoid dendritic cells (PDC), while TLR8 induces the secretion of high amounts of IL-12p70 in myeloid immune cells. It has been demonstrated in the art that TLR8 selectively detects ssRNA, while TLR7 primarily detects short stretches of dsRNA but can also accommodate certain ssRNA oligonucleotides. TLR9 receptors are predominantly expressed in human B cells and plasmacytoid dendritic cells and detect single-stranded DNA containing unmethylated CpG dinucleotides. Additionally to the induction of cytokines, some RNA sensing pattern recognition receptors of the innate immune system can inhibit protein translation upon binding of its agonist (e.g. dsRNA, 5′ ppp RNA), such as e.g. PKR and OAS1. For example, binding of a long double-stranded RNA is taught to activate PKR to phosphorylate eIF2a leading to inhibition of translation of an mRNA molecule. IFIT1 and IFIT5 is taught to bind to 5′ ppp RNA leads to a blockade of eIF2a, thereby inhibiting translation of an mRNA molecule (reviewed in Hartmann, G. “Nucleic acid immunity.” Advances in immunology. Vol. 133. Academic Press, 2017. 121-169).

Accordingly, in the context of the invention, the term “RNA sensing pattern recognition receptor” as used herein refers to a class of PRRs capable to sense RNA. “Sense” in that context has to be understood as the capability of a receptor to bind to the RNA, and, in consequence, to trigger downstream signaling cascades (e.g. induction of cytokines or e.g. inhibition of translation).

Accordingly, the term “antagonist of at least one RNA sensing pattern recognition receptor” relates to a compound capable of inhibiting and/or suppressing a PRRs-mediated immune response induced by the therapeutic RNA of the invention. Further, such an antagonist may attenuate the effects (e.g. PRRs-mediated immune response) of an agonist (e.g. immune stimulating RNA species).

Accordingly, the at least one RNA sensing pattern recognition receptor preferably induces cytokines upon binding of an RNA agonist. Such an RNA agonist may be a single stranded RNA, a double stranded RNA, or a 5′ triphosphated RNA (5′ ppp RNA).

Alternatively or in addition, the at least one RNA sensing pattern recognition receptor may inhibit translation upon binding of an RNA agonist. Such an RNA agonist may be a single stranded, double stranded, or a 5′ triphosphated RNA (5′ ppp RNA).

Advantageously, the at least one antagonist of the second component reduces the cytokine induction of the at least one RNA sensing pattern recognition receptor upon binding of an RNA agonist and/or reduces translation inhibition by the at least one RNA sensing pattern recognition receptor upon binding of an RNA agonist. Accordingly, in preferred embodiments, administration of the combination of the at least one therapeutic RNA of the first component and the at least one antagonist of at least one RNA sensing pattern recognition receptor of the second component leads to a reduced innate immune response compared to administration of the at least one therapeutic RNA of the first component without combination with the at least one antagonist of at least one RNA sensing pattern recognition receptor of the second component.

Accordingly, administration of the combination (that is, administration of the first and the second component) to a cell, tissue, or organism results in a reduced (innate) immune stimulation as compared to administration of the corresponding first component only.

In further embodiments, administration of the combination (that is, administration of the first and the second component) to a cell, tissue, or organism results in essentially the same or at least a comparable (innate) immune stimulation as compared to administration of a control RNA comprising modified nucleotides (e.g. as defined herein) and having the same RNA sequence.

The induction or activation or stimulation of an innate immune response as described above is usually determined by measuring the induction of cytokines.

Preferably, reduced innate immune stimulation is characterized by a reduced level of at least one cytokine preferably selected from Rantes, MIP-1 alpha, MIP-1 beta, McP1, TNFalpha, IFNgamma, IFNalpha, IFNbeta, IL-12, IL-6, or IL-8.

The term “reduced level of at least one cytokine” has to be understood as that the administration of the combination according to the invention reduces the induction of cytokines compared to a control (e.g. first component only) to a certain percentage.

Accordingly, reduced innate immune stimulation in the context of the invention is characterized by a reduced level of at least one cytokine preferably selected from Rantes, MIP-1 alpha, MIP-1 beta, McP1, TNFalpha, IFNgamma, IFNalpha, IFNbeta, IL-12, IL-6, or IL-8, wherein the reduced level of at least one cytokine is a reduction of at least 10%, 15%, 20%, 25%, 30%, 35%, 40%, 45%, 50%, 55%, 60%, 65%, 70%, 75%, 80%, 85%, 90%, or 95%. Preferably, the reduced level of at least one cytokine is a reduction of at least 30%.

Methods to evaluate the (innate) immune stimulation (that is, the induction of e.g. Rantes, MIP-1 alpha, MIP-1 beta, McP1, TNFalpha, IFNgamma, IFNalpha, IFNbeta, IL-12, IL-6, or IL-8) by the therapeutic RNA in specific cells/organs/tissues are well known in the art for the skilled artisan. Typically, (innate) immune stimulation of the therapeutic RNA in combination with the second component is compared with the (innate) immune stimulation of the therapeutic RNA alone (or with a control RNA comprising modified nucleotides), that is, without the (additional) administration of the second component. The same conditions (e.g. the same cell lines, same organism, same application route, the same detection method, the same amount of therapeutic RNA, the same RNA sequence etc.) have to be used (if feasible) to allow a valid comparison. The person of skill in the art understands how to perform a comparison of the inventive combination and a respective control RNA (therapeutic RNA alone or control RNA comprising modified nucleotides and having the same RNA sequence).

In the context of the invention, the induction of cytokines is measured by administration of the combination into cells, a tissue or an organism, preferably hPBMCs, Hela cells or HEK cells. Preferred in that context are hPBMCs. Upon administration of the combination (or the corresponding control) to hPBMCs, Hela cells or HEK cells, an assay for measuring cytokine levels is performed. Cytokines secreted into culture media or supernatants can be quantified by techniques such as bead based cytokine assays (e.g. cytometric bead array (CBA)), ELISA, and Western blot.

Preferably, a bead based cytokine assays, most preferably a cytometric bead array (CBA) is performed to measure the induction of cytokines in cells after administration of the combination (and their corresponding controls).

CBA can quantify multiple cytokines from the same sample. The CBA system uses a broad range of fluorescence detection offered by flow cytometry and antibody-coated beads to capture cytokines. Each bead in the array has a unique fluorescence intensity so that beads can be mixed and acquired simultaneously. A suitable CBA assay in that context is described in a BD Bioscience application note of 2012, “Quantification of Cytokines Using BD™ Cytometric Bead Array on the BD™ FACSVerse System and Analysis in FCAP Array™ Software”, from Reynolds et al. An exemplary CBA assay for determining cytokine levels is described in the examples section of the present invention.

In various embodiments, the at least one RNA sensing pattern recognition receptor is an endosomal receptor or a cytoplasmic receptor. In preferred embodiments the at least one RNA sensing pattern recognition receptor is an endosomal receptor. A non-limiting list of exemplary endosomal RNA sensing pattern recognition receptors comprises TLR3, TLR7, or TLR8. In that context, “endosomal” has to be understood as localized in the endosome or localized in the endosomal membrane. A non-limiting list of exemplary cytoplasmic RNA sensing pattern recognition receptors comprises RIG1, MDA5, NLRP3, or NOD2.

In various embodiments, the at least one RNA sensing pattern recognition receptor is a receptor for single stranded RNA (ssRNA) and/or a receptor for double stranded RNA (dsRNA). A non-limiting list of exemplary RNA sensing pattern recognition receptors for dsRNA comprises TLR3, RIG1, MDA5, NLRP3, or NOD2. A non-limiting list of exemplary RNA sensing pattern recognition receptors for ssRNA comprises TRL7, TLR8, RIG1, NLRP3, or NOD2.

Accordingly, in preferred embodiments, the at least one second component comprises at least one antagonist of at least one RNA sensing pattern recognition receptor, wherein at least one RNA sensing pattern recognition receptor is selected from a Toll-like receptor (TLR), and/or a Retinoic acid-inducible gene-I-like receptor (RLR), and/or a NOD-like receptor and/or PKR, OAS, SAMHD1, ADAR1, IFIT1 and/or IFIT5.

In preferred embodiments, the at least one second component comprises at least one antagonist of at least one RNA sensing pattern recognition receptor, wherein at least one RNA sensing pattern recognition receptor is selected from PKR, OAS, SAMHD1, ADAR1, IFIT1 and/or IFIT5.

In preferred embodiments, the at least one Toll-like receptor is selected from TLR3, TLR7, TLR8 and/or TLR9. In particularly preferred embodiments, the Toll-like receptor is selected from TLR7 and/or TLR8. Accordingly in the context of the invention, it is preferred that “the at least one antagonist of at least one RNA sensing pattern recognition receptor” is an antagonist of a Toll-like receptor selected from TLR3, TLR7, TLR8 and/or TLR9, preferably TLR7 and/or TLR8.

In preferred embodiments, the at least one retinoic acid-inducible gene-I-like receptor (RLR) is selected from RIG-1, MDA5, LGP2, cGAS, AIM2, NLRP3, and/or NOD2. In particularly preferred embodiments, the RLR is RIG-1 and/or MDA5. Accordingly in the context of the invention, it is preferred that “the at least one antagonist of at least one RNA sensing pattern recognition receptor” is an antagonist of a retinoic acid-inducible gene-I-like receptor (RLR) selected from RIG-1, MDA5, LGP2, cGAS, AIM2, NLRP3, and/or NOD2, preferably RIG-1, MDA5.

In the context of the invention, the at least one antagonist of the second component as defined herein may be selected from a nucleotide, a nucleotide analogue, a nucleic acid, a peptide, a protein, an antibody, a small molecule, a lipid, or a fragment, variant, or derivative of any of these.

In some embodiments, the antagonist is a TLR antagonist including substituted quinoline compounds, substituted quinazole compounds, tricyclic TLR inhibitors (e.g., mianserin, desipramine, cyclobenzaprine, imiprimine, ketotifen, and amitriptyline), Vaccinia virus A52R protein (US 20050244430), Polymyxin-B (specific inhibitor of LPS-bioactivity), BX795, chloroquine, hydroxychloroquine, CU-CPT8m, CU-CPT9a, CU-CPT9b, CU-CPT9c, CU-CPT9d, CU-CPT9e, CU-CPT9f, CLI-095, RDP58, ST2825, ML120B, PHA-408, insulin (Clinical trial NCTO1 151605), oligodeoxynucleotides (ODN) that suppress CpG-induced immune responses, G-rich ODN, and ODN with TTAGGG motifs. In some embodiments, TLR antagonists include those described in patents or patent applications US20050119273, WO2014052931, WO2014108529, US20140094504, US20120083473, U.S. Pat. No. 8,729,088 and US20090215908. In some embodiments, TLR inhibitors include ST2 antibody; sST2-Fc (functional murine soluble ST2-human IgGI Fc fusion protein; see Biochemical and Biophysical Research Communications, 29 Dec. 2006, vol. 351, no. 4, 940-946); CRX-526 (Corixa); lipid IVA; RSLA (Rhodobacter sphaeroides lipid A); E5531 ((6-0-{2-deoxy-6-0-methyl-4-0-phosphono-3-0-[(R)-3-Z-dodec-5-endoyloxydecl]-2-[3-oxo-tetradecanoylamino]-0- phosphono-a-D-glucopyranose tetrasodium salt); E5564 (a-D-Glucopyranose,3-0-decyl-2- deoxy-6-0-[2-deoxy-3-0-[(3R)-3-methoxydecyl]-6-0- methyl-2- [[(11 Z)-1-oxo-11-octadecenyl] amino]-4-0-phosphono-D-glucopyranosyl]-2-[(1,3-dioxotetradecyl)amino]-l-(dihydrogen phosphate), tetrasodium salt); compound 4a (hydrocinnamoyl-L-valyl pyrrolidine; see PNAS, Jun. 24, 2003, vol. 100, no. 13, 7971-7976); CPG 52364 (Coley Pharmaceutical Group); LY294002 (2-(4-Morpholinyl)-8-phenyl-4H-1-benzopyran-4-one); PD98059 (2-(2-amino-3-methoxyphenyl)-4H-1-Benzopyran-4-one); chloroquine; (C2 dimer with a propylene spacer as antagonist of TLR7/8 (see Table A) and an immune regulatory oligonucleotide (see U.S. Patent Application Publication No. 2008/0089883). Further suitable TLR antagonists are described by Patinote et al (Patinote et al, Agonist and antagonist ligands of toll-like receptors 7 and 8: Ingenious tools for therapeutic purposes, Eur J Med Chem. 2020 May 1; 193: 112238.)

Accordingly, suitable chemical compounds, e.g. small molecule compounds that may be used as antagonist in the context of the invention may be selected from Chloroquine, CU-CPT9a, Hydroxychloroquin, quinacrine, monesin, bafilomycin Ai, wortmannin, β-aminoarteether maleate, (+)-morphinans, 9-aminoacridine, 4-aminoquinoline, 4-aminoquinolines, 7,8,9, 10-tetrahydro-6H-cyclohepta[b]quinolin-I 1- ylamine; 1-methyl-2,3-dihydro-IH-pyrrolo[2,3-b]quinolin-4-ylamine; 1,6-dimethyl-2,3- dihydro- IH-pyrrolo[2,3-b]quinolin-4-ylamine; 6-bromo-1-methyl-2,3-dihydro- 1H-pyrrolo[2,3-b]quinolin-4-ylamine; 1-methyl-2,3,4,5-tetrahydro-IH-azepino[2,3-b]quinolin-6- ylamine; 3,3-dimethyl-3,4-dihydro-acridin-9-ylamine; 1-benzyl-2,3-dihydro-IH-pyrrolo[2,3-b]quinolin-4-ylamine; 6-methyl-1-phenyl-2,3-dihydro-1 H-pyrrolo[2,3-b]quinolin-4-ylamine; N*2*,N*2*-Dimethyl-quinoline-2,4-diamine, 2,7-Dimethyl-dibenzo[b,g][1,8]naphthyridin-11-ylamine; 2,4-Dimethyl-benzo[b][I,8]naphthyridin-5-ylamine; 7-Fluoro-2,4-dimethyl- benzo[b][I,8]naphthyridin-5-ylamine; 1,2,3,4-Tetrahydro-acridin-9-ylamine Tacrine hydrochloridehydrate; 2,3-Dihydro-IH-cyclopenta[b]quinolin-9-ylamine; 2,4,9-Trimethyl- benzo[b][I,8]naphthyridin-5-ylamine; 9-Amino-3,3-dimethyl-I,2,3,4-tetrahydro-acridin-1-ol and 7-Ethoxy-N*3*-furan-2-ylmethyl-acridine-3,9-diamine; quinazolines, N,N-dimethyl-N′-{2-[4-(4-methyl-piperazin-1-yl)-phenyl]-3,4-dihydro-quinazoline-4-yl}-ethane- 1,2,-diamine; N′-[6,7-Dimethoxy-2-(4-phenyl-piperazin-1-yl)-quinazolin-4-yl]-N,N-dimethyl-ethane-1,2- diamine; N′-[6,7-Dimethoxy-2-(4-methyl-piperazin-1-yl)-quinazolin-4-yl]-N,N-dimethyl-ethane-1,2-diamine; N,N-Dimethyl-N′-(2-phenyl-quinazolin-4-yl)-ethane- 1,2-diamine; Dimethyl-(2-{2-[4-(4-methyl-piperazin-1-yl)-phenyl]-quinazolin-4-yloxy}-ethyl)-amine; N′-(2-Biphenyl-4-yl-quinazolin-4-yl)-N,N-dimethyl-ethane-I,2-diamine and Dimethyl-[2-(2- phenyl-quinazolin-4-yloxy)-ethyl]-amine, statins, atorvastatin.

In some embodiments the suitable chemical compounds, e.g. small molecule compounds may be selected from Chloroquine (C₁₈H₂₆ClN₃), an antimalarial medicine with anti-inflammatory, and potential chemosensitization and radiosensitization activities or CU-CPT9a (C₁₇H₁₅NO₂), which is potent and selective inhibitor of Toll-like receptor 8 (see Table A), (Zhang, S. et al, 2018. Small-molecule inhibition of TLR8 through stabilization of its resting state. Nat Chem Biol, 14(1): 58-64 and Mohamed et al, effect of toll-like receptor 7 and 9 targeted therapy to prevent the development of hepatocellular carcinoma, Liver International (2015).

TABLE A
Preferred small molecule antagonists of the invention:

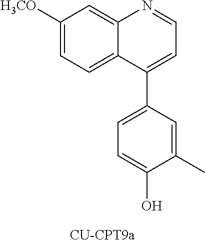 [SMILES: COc1ccc2c(-c3ccc(O)c(C)c3)ccnc2c1]

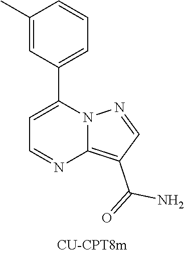 [SMILES: Cc1cccc(-c2ccnc3c(C(N)=O)cnn23)c1]

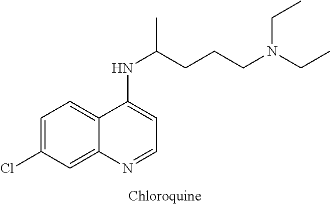 [SMILES: CCN(CC)CCCC(C)Nc1ccnc2cc(Cl)ccc12]

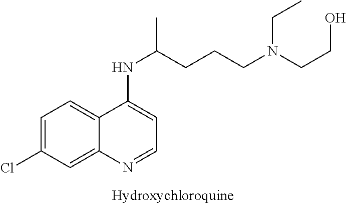 [SMILES: CCN(CCO)CCCC(C)Nc1ccnc2cc(Cl)ccc12]

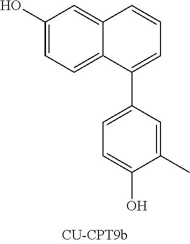 [SMILES: Cc1cc(-c2cccc3cc(O)ccc23)ccc1O]

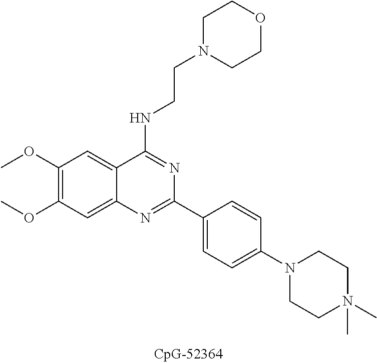

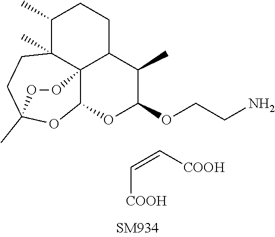

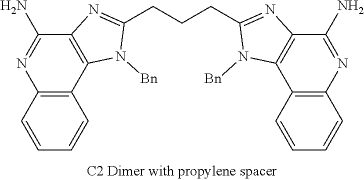 [SMILES: Nc1nc2ccccc2c2c1nc(CCCc1nc3c(N)nc4ccccc4c3n1Cc1ccccc1)n2Cc1ccccc1]

In preferred embodiments, the “at least one antagonist of at least one RNA sensing pattern recognition receptor” of the second component of the combination is a nucleic acid.

The terms “nucleic acid” or “nucleic acid molecule” will be recognized and understood by the person of ordinary skill in the art, and are e.g. intended to refer to a molecule comprising, preferably consisting of nucleic acid components. The term nucleic acid molecule preferably refers to DNA and RNA or mixtures thereof. It is preferably used synonymous with the term polynucleotide. Preferably, a nucleic acid or a nucleic acid molecule is a polymer comprising or consisting of nucleotide monomers (natural and/or modified), which are covalently linked to each other by phosphodiester-bonds of a sugar/phosphate-backbone. An example of suitable modified nucleotide are LNA or PNA nucleotides. The term “nucleic acid” also encompasses modified nucleic acid molecules, such as base-modified, sugar-modified or backbone-modified DNA or RNA molecules as defined herein. The term “nucleic acid” also encompasses single stranded, double stranded, and branched nucleic acid molecules.

In particularly preferred embodiments, the “at least one antagonist of at least one RNA sensing pattern recognition receptor” of the second component of the combination is a single stranded nucleic acid, for example a single stranded RNA.

In alternative embodiments, the “at least one antagonist of at least one RNA sensing pattern recognition receptor” of the second component of the combination is a double stranded nucleic acid, for example a double stranded RNA.

In preferred embodiments, the “at least one antagonist of at least one RNA sensing pattern recognition receptor” of the second component of the combination is a nucleic acid comprising or consisting of nucleotides selected from DNA nucleotides, RNA nucleotides, PNA nucleotides, and/or LNA nucleotides, or analogs, or derivatives of any of these.

In particularly preferred embodiments, the “at least one antagonist of at least one RNA sensing pattern recognition receptor” of the second component of the combination is a single stranded nucleic acid, wherein said nucleic acid comprises or consists of nucleotides selected from DNA nucleotides, RNA nucleotides, PNA nucleotides, and/or LNA nucleotides, or analogs of any of these.

In other embodiments, the “at least one antagonist of at least one RNA sensing pattern recognition receptor” of the second component of the combination is a double stranded nucleic acid, wherein said nucleic acid comprises or consists of nucleotides selected from DNA nucleotides, RNA nucleotides, PNA nucleotides, and/or LNA nucleotides, or analogs of any of these.

The term “LNA nucleotide” as used herein refers to a modified RNA nucleotide. A LNA nucleotide is a locked nucleic acid. The ribose moiety of an LNA nucleotide may be modified with an extra bridge connecting the 2′ oxygen and 4′ carbon. This bridge locks the ribose in the 3′-endo (North) conformation, which is often found in the A-form duplexes. LNA nucleotides can be mixed with DNA or RNA residues in an e.g. oligonucleotide. LNA nucleotides hybridize with DNA or RNA. Oligomers comprising LNA nucleotides are synthesized chemically and are commercially available. The locked ribose conformation enhances base stacking and backbone pre-organization.

The term “PNA nucleotide” as used herein refers to a modified nucleic acid. DNA and RNA have a deoxyribose and ribose sugar backbone. The backbone of PNA is composed of repeating N-(2-aminoethyl)-glycine units and it is linked by peptide bonds. Therefore, PNAs are depicted like peptides, i.e. from N-terminus to C-terminus.

PNAs exhibit a higher binding strength. PNA oligomers also show greater specificity in binding to complementary DNAs, with a PNA/DNA base mismatch being more destabilizing than a similar mismatch in a DNA/DNA duplex. This binding strength and specificity also applies to PNA/RNA duplexes. PNAs are not easily recognized by either nucleases or proteases and PNAs are also stable over a wide pH range.

In specific embodiments, the nucleic acid of the second component is a hybrid RNA nucleic acid, wherein said hybrid RNA nucleic acid comprises RNA nucleotides and, additionally at least one DNA, LNA, or PNA nucleotide.

In specific embodiments, the nucleic acid comprises at least one modified nucleotide and/or at least one nucleotide analogue or nucleotide derivative.

The terms “analog” or “derivative” can be used interchangeably to generally refer to any purine and/or pyrimidine nucleotide or nucleoside that has a modified base and/or sugar. A modified base is a base that is not guanine, cytosine, adenine, thymine or uracil. A modified sugar is any sugar that is not ribose or 2′deoxyribose and can be used in the backbone for an oligonucleotide.

In embodiments, the nucleic acid of the second component comprises at least one modified nucleotide and/or at least one nucleotide analogue, wherein the at least one modified nucleotide and/or at least one nucleotide analogue is selected from a backbone modified nucleotide, a sugar modified nucleotide and/or a base modified nucleotide or any combinations thereof.

A backbone modification in the context of the invention is a modification in which phosphates of the backbone of the nucleotides are chemically modified. A sugar modification in the context of the invention is a chemical modification of the sugar of the nucleotides. A base modification in the context of the invention is a chemical modification of the base moiety of the nucleotides.

In embodiments, the nucleotide analogues/modifications which may be incorporated into the nucleic acid of the second component as described herein are preferably selected from 2-amino-6-chloropurineriboside-5′-triphosphate, 2-Aminopurine-riboside-5′-triphosphate; 2-aminoadenosine-5′-triphosphate, 2′-Amino-2′-deoxycytidine-triphosphate, 2-thiocytidine-5′-triphosphate, 2-thiouridine-5′-triphosphate, 2′-Fluorothymidine-5′-triphosphate, 2′-O-Methyl-inosine-5′-triphosphate 4-thiouridine-5′-triphosphate, 5-aminoallylcytidine-5′-triphosphate, 5-aminoallyluridine-5′-triphosphate, 5-bromocytidine-5′-triphosphate, 5-bromouridine-5′-triphosphate, 5-Bromo-2′-deoxycytidine-5′-triphosphate, 5-Bromo-2′-deoxyuridine-5′-triphosphate, 5-iodocytidine-5′-triphosphate, 5-Iodo-2′-deoxycytidine-5′-triphosphate, 5-iodouridine-5′-triphosphate, 5-Iodo-2′-deoxyuridine-5′-triphosphate, 5-methylcytidine-5′-triphosphate, 5-methyluridine-5′-triphosphate, 5-Propynyl-2′-deoxycytidine-5′-triphosphate, 5-Propynyl-2′-deoxyuridine-5′-triphosphate, 6-azacytidine-5′-triphosphate, 6-azauridine-5′-triphosphate, 6-chloropurineriboside-5′-triphosphate, 7-deazaadenosine-5′-triphosphate, 7-deazaguanosine-5′-triphosphate, 8-azaadenosine-5′-triphosphate, 8-azidoadenosine-5′-triphosphate, benzimidazole-riboside-5′-triphosphate, N1-methyladenosine-5′-triphosphate, N1-methylguanosine-5′-triphosphate, N6-methyladenosine-5′-triphosphate, 06-methylguanosine-5′-triphosphate, pseudouridine-5′-triphosphate, or puromycin-5′-triphosphate, xanthosine-5′-triphosphate. Particular preference is given to nucleotides for base modifications selected from the group of base-modified nucleotides consisting of 5-methylcytidine-5′-triphosphate, 7-deazaguanosine-5′-triphosphate, 5-bromocytidine-5′-triphosphate, and pseudouridine-5′-triphosphate, pyridin-4-one ribonucleoside, 5-aza-uridine, 2-thio-5-aza-uridine, 2-thiouridine, 4-thio-pseudouridine, 2-thio-pseudouridine, 5-hydroxyuridine, 3-methyluridine, 5-carboxymethyl-uridine, 1-carboxymethyl-pseudouridine, 5-propynyl-uridine, 1-propynyl-pseudouridine, 5-taurinomethyluridine, 1-taurinomethyl-pseudouridine, 5-taurinomethyl-2-thio-uridine, 1-taurinomethyl-4-thio-uridine, 5-methyl-uridine, 1-methyl-pseudouridine, 4-thio-1-methyl-pseudouridine, 2-thio-1-methyl-pseudouridine, 1-methyl-1-deaza-pseudouridine, 2-thio-1-methyl-1-deaza-pseudouridine, dihydrouridine, dihydropseudouridine, 2-thio-dihydrouridine, 2-thio-dihydropseudouridine, 2-methoxyuridine, 2-methoxy-4-thio-uridine, 4-methoxy-pseudouridine, and 4-methoxy-2-thio-pseudouridine, 5-aza-cytidine, pseudoisocytidine, 3-methyl-cytidine, N4-acetylcytidine, 5-formylcytidine, N4-methylcytidine, 5-hydroxymethylcytidine, 1-methyl-pseudoisocytidine, pyrrolo-cytidine, pyrrolo-pseudoisocytidine, 2-thio-cytidine, 2-thio-5-methyl-cytidine, 4-thio-pseudoisocytidine, 4-thio-1-methyl-pseudoisocytidine, 4-thio-1-methyl- 1-deaza-pseudoisocytidine, 1-methyl-1-deaza-pseudoisocytidine, zebularine, 5-aza-zebularine, 5-methyl-zebularine, 5-aza-2-thio-zebularine, 2-thio-zebularine, 2-methoxy-cytidine, 2-methoxy-5-methyl-cytidine, 4-methoxy-pseudoisocytidine, and 4-methoxy-1-methyl-pseudoisocytidine, 2-aminopurine, 2, 6-diaminopurine, 7-deaza-adenine, 7-deaza-8-aza-adenine, 7-deaza-2-aminopurine, 7-deaza-8-aza-2-aminopurine, 7-deaza-2,6-diaminopurine, 7-deaza-8-aza-2,6-diaminopurine, 1-methyladenosine, N6-methyladenosine, N6-isopentenyladenosine, N6-(cis-hydroxyisopentenyl)adenosine, 2-methylthio-N6-(cis-hydroxyisopentenyl) adenosine, N6-glycinylcarbamoyladenosine, N6-threonylcarbamoyladenosine, 2-methylthio-N6-threonyl carbamoyladenosine, N6,N6-dimethyladenosine, 7-methyladenine, 2-methylthio-adenine, and 2-methoxy-adenine, inosine, 1-methyl-inosine, wyosine, wybutosine, 7-deaza-guanosine, 7-deaza-8-aza-guanosine, 6-thio-guanosine, 6-thio-7-deaza-guanosine, 6-thio-7-deaza-8-aza-guanosine, 7-methyl-guanosine, 6-thio-7-methyl-guanosine, 7-methylinosine, 6-methoxy-guanosine, 1-methylguanosine, N2-methylguanosine, N2,N2-dimethylguanosine, 8-oxo-guanosine, 7-methyl-8-oxo-guanosine, 1-methyl-6-thio-guanosine, N2-methyl-6-thio-guanosine, and N2,N2-dimethyl-6-thio-guanosine, 5′-O-(1-thiophosphate)-adenosine, 5′-O-(1-thiophosphate)-cytidine, 5′-O-(1-thiophosphate)-guanosine, 5′-O-(1-thiophosphate)-uridine, 5′-O-(1-thiophosphate)-pseudouridine, 6-aza-cytidine, 2-thio-cytidine, alpha-thio-cytidine, Pseudo-iso-cytidine, 5-aminoallyl-uridine, 5-iodo-uridine, N1-methyl-pseudouridine, 5,6-dihydrouridine, alpha-thio-uridine, 4-thio-uridine, 6-aza-uridine, 5-hydroxy-uridine, deoxy-thymidine, 5-methyl-uridine, Pyrrolo-cytidine, inosine, alpha-thio-guanosine, 6-methyl-guanosine, 5-methyl-cytdine, 8-oxo-guanosine, 7-deaza-guanosine, N1-methyl-adenosine, 2-amino-6-Chloro-purine, N6-methyl-2-amino-purine, Pseudo-iso-cytidine, 6-Chloro-purine, N6-methyl-adenosine, alpha-thio-adenosine, 8-azido-adenosine, 7-deaza-adenosine.

In preferred embodiments, the at least one modified nucleotide and/or the at least one nucleotide analogue is selected from a modified nucleotide found in bacterial tRNA. In particularly preferred embodiments, the at least one modified nucleotide and/or the at least one nucleotide analogue is selected from 1-methyladenosine, 2-methyladenosine, N6-methyladenosine, 2′-O-methyladenosine, 2-methylthio-N6-methyladenosine, N6-isopentenyladenosine, 2-methylthio-N6-isopentenyladenosine, N6-threonylcarbamoyladenosine, 2-methylthio-N6-threonyl carbamoyladenosine, N6-methyl-N6-threonylcarbamoyladenosine, N6-hydroxynorvalylcarbamoyladenosine, 2-methylthio-N6-hydroxynorvalyl carbamoyladenosine, inosine, 3-methylcytidine, 2′-O-methylcytidine, 2-thiocytidine, N4-acetylcytidine, lysidine, 1-methylguanosine, 7-methylguanosine, 2′-O-methylguanosine, queuosine, epoxyqueuosine, 7-cyano-7-deazaguanosine, 7-aminomethyl-7-deazaguanosine, pseudouridine, dihydrouridine, 5-methyluridine, 2′-O-methyluridine, 2-thiouridine, 4-thiouridine, 5-methyl-2-thiouridine, 3-(3-amino-3-carboxypropyl)uridine′, 5-hydroxyuridine, 5-methoxyuridine, uridine 5-oxyacetic acid, uridine 5-oxyacetic acid methyl ester, 5-aminomethyl-2-thiouridine, 5-methylaminomethyluridine, 5-methylaminomethyl-2-thiouridine, 5-methylaminomethyl-2-selenouridine, 5-carboxymethylaminomethyluridine, 5-carboxymethylaminomethyl- 2′-O-methyluridine, 5-carboxymethylaminomethyl-2-thiouridine, 5-(isopentenylaminomethyl)uridine, 5-(isopentenylaminomethyl)- 2-thiouridine, 5-(isopentenylaminomethyl)- 2′-O-methyluridine.

In preferred embodiments, the nucleic acid of the second component comprises at least one 2′-substituted RNA nucleotide (ribonucleoside).

The term “2′-substituted ribonucleoside” generally includes ribonucleosides in which the hydroxyl group at the 2′ position of the pentose moiety is substituted to produce a 2′-substituted or 2′-O-substituted ribonucleoside. In certain embodiments, such substitution is with a lower hydrocarbyl group containing 1-6 saturated or unsaturated carbon atoms, with a halogen atom, or with an aryl group having 6-10 carbon atoms, wherein such hydrocarbyl, or aryl group may be unsubstituted or may be substituted, e.g., with halo, hydroxy, trifiuoromethyl, cyano, nitro, acyl, acyloxy, alkoxy, carboxyl, carboalkoxy, or amino groups.

In preferred embodiments, the nucleic acid of the second component comprises at least one sugar modified nucleotide. Preferably, said sugar modified nucleotide is at least one 2′ Ribose modified (ribonucleoside) RNA nucleotide.

Examples of 2′-O-substituted ribonucleosides include, without limitation 2′-amino, 2′-fluoro, 2′-allyl, 2′-O-alkyl and 2′-propargyl ribonucleosides, 2′-O-methylribonucleosides and 2′-O-methoxyethoxyribonucleosides.

In particularly preferred embodiments, the at least one 2′ Ribose modified RNA nucleotide of the nucleic acid of the second component is a 2′-O-methylated RNA nucleotide (2′-O-methylribonucleotide).

In particularly preferred embodiments, the nucleic acid of the second component comprises at least one 2′ Ribose modified RNA nucleotide, wherein said at least one 2′ Ribose modified RNA nucleotide is a 2′-O-methylated RNA nucleotide. Preferably, 2′-O-methylated RNA nucleotides may be selected from 2′-O-methylated guanosine (Gm), 2′-O-methylated uracil (Um), 2′-O-methylated adenosine (Am), 2′-O-methylated cytosine (Cm), or a 2′-O-methylated analog of any of these nucleotides.

In particularly preferred embodiments, the nucleic acid of the second component comprises at least one 2′-O-methylated RNA nucleotide, preferably at least 2, 3, 4, 5, 6, 7, 8, 9, 10 or more 2′-O-methylated RNA nucleotides, wherein said at least one or said at least 2, 3, 4, 5, 6, 7, 8, 9, 10 or more 2′-O-methylated RNA nucleotides may be selected from 2′-O-methylated guanosine (Gm), 2′-O-methylated uracil (Um), 2′-O-methylated adenosine (Am), 2′-O-methylated cytosine (Cm), or a 2′-O-methylated analog of any of these nucleotides.

In preferred embodiments, the nucleic acid of the second component comprises at least one 2′-O-methylated RNA nucleotide, wherein, preferably, the at least one 2′-O-methylated RNA nucleotide is not located at the 5′ terminal end and/or the 3′ terminal end of the nucleic acid.

In preferred embodiments, the nucleic acid of the second component comprises at least one or more of a trinucleotide M-X-Y motifs,

wherein M is selected from Gm, Um, or Am, preferably wherein M is Gm;

wherein X is selected from G, A, or U, preferably wherein X is G or A; and

wherein Y is selected from G, A, U, C, or dihydrouridine, preferably wherein Y is C.

In particularly preferred embodiments, the nucleic acid of the second component comprises at least one or more of a trinucleotide M-X-Y motifs,

wherein M is Gm;

wherein X is G or A; and

wherein Y is C.

In particular embodiments, the nucleic acid of the second component comprises at least 2, 3, 4, 5, 6, 7, 8, 9, 10 or more trinucleotide M-X-Y motifs as defined herein, wherein each M-X-Y motif may be independently defined as described herein.

In particular embodiments, the nucleic acid of the second component comprises at least 2, 3, 4, 5, 6, 7, 8, 9, 10 or more trinucleotide M-X-Y motifs as defined herein, wherein said trinucleotide motif is not located at the 3′ terminus and/or the 5′ terminus.

In particularly preferred embodiments, the nucleic acid of the second component comprises or consists of at least one nucleic acid sequence according to formula I:

N_{W}-M-X-Y-N_{Z} (Formula I)

wherein N is independently selected from any nucleotide or nucleotide analog as defined herein, preferably G, A, U, C, Gm, Am, Um, Cm, or a modified nucleotide as defined herein;

wherein W is O or an integer of 1 to 15, preferably wherein W is an integer of 1 to 10, most preferably 1 to 5;

wherein Z is 0 or an integer of 1 to 15, preferably wherein Z is an integer of 1 to 10, most preferably 1 to 5;

wherein M, X, and Y are selected as defined herein.

In particularly preferred embodiments, the nucleic acid of the second component comprises or consists of at least one nucleic acid sequence according to formula (i),

wherein N is independently selected from G, A, U, C;

wherein W is an integer of 1 to 10;

wherein Z is an integer of 1 to 10;

wherein M is Gm;

wherein X is G;

and wherein Y is C.

Exemplary nucleic acid sequences that may be derived from Formula I are:

5′-MXYNNNNNNNN-3′

5′-NMXYNNNNNNN-3′

5′-NNMXYNNNNNN-3′

5′-NNNMXYNNNNN-3′

5′-NNNNMXYNNNN-3′

5′-NNNNNMXYNNN-3′

5′-NNNNNNMXYNN-3′

5′-NNNNNNNMXYN-3′

5′-NNNNNNNNMXY-3′

5′-MXYNNNNNNN-3′

5′-NMXYNNNNNN-3′

5′-NNMXYNNNNN-3′

5′-NNNMXYNNNN-3′

5′-NNNNMXYNNN-3′

5′-NNNNNMXYNN-3′

5′-NNNNNNMXYN-3′

5′-NNNNNNNMXY-3′

5′-MXYNNNNNN-3′

5′-NMXYNNNNN-3′

5′-NNMXYNNNN-3′

5′-NNNMXYNNN-3′

5′-NNNNMXYNN-3′

5′-NNNNNMXYN-3′

5′-NNNNNNMXY-3′

5′-MXYNNNNN-3′

5′-NMXYNNNN-3′

5′-NNMXYNNN-3′

5′-NNNMXYNN-3′

5′-NNNNMXYN-3′

5′-NNNNNMXY-3′

etc.

In particularly preferred embodiments, the nucleic acid of the second component comprises or consists of at least 2, 3, 4, 5, 6, 7, 8, 9, 10 or more nucleic acid sequences according to formula I, wherein each of the at least 2, 3, 4, 5, 6, 7, 8, 9, 10 or more nucleic acid sequences according to formula I may be identical or may be independently selected from each other.

In that context, exemplary nucleic acid sequences that may be derived from Formula I are:

5′-NNNNNMXYMXYNNNNNNNNNNNMXYN-3′

5′-NNNNNMXYMXYNNNNNNNNMXYN-3′

5′-NNMXYNNNNNMXYNNNMXYNNN-3′

5′-NNNMXYMXYNNNNNNNNNMXYN-3′

5′-NNMXYNNNMXYNNNMXYNNN-3′

5′-NNNMXYMXYNNNNNNMXYN-3′

5′-MXYNNNNNNNNNNNNNMXY-3′

5′-MXYNNNNNNNNNNNNNMXY-3′

5′-NNMXYNNNNNMXYNNNMN-3′

5′-MXYNNNNNNNNNNNMXY-3′

5′-NNMXYNNNMXYNNNNN-3′

5′-MXYNNNNNNNNNMXY-3′

etc.

In particularly preferred embodiments, the nucleic acid of the second component contains a 5′ end that is devoid of a triphosphate group. In other words, the 5′ end of the nucleic acid of the second component may comprise a monophosphate group or a diphosphate group or a hydroxyl group. It is particularly important in the context of the invention that the nucleic acid of the second component is lacking a 5′ terminal triphosphate group, as such a 5′ ppp group potentially stimulates the innate immune response upon administration (via RIG-1).

Accordingly, in embodiments the nucleic acid of the second component is generated using synthetic methods (e.g. RNA synthesis). In embodiments where the nucleic acid of the second component is generated using enzymatic processes (e.g. RNA in vitro transcription), it may be required to remove the 5′ppp group of the nucleic acid to obtain a nucleic acid that contains a 5′ end that is devoid of a triphosphate group (e.g. using a phosphatase treatment).

In alternative embodiments, the nucleic acid of the second component contains a triphosphate group at the 5′ end, wherein such a 5′ triphosphate group containing nucleic acid may be generated using synthetic methods or enzymatic processes.

The nucleic acid of the second component may have a length of 1 to about 200 nucleotides, about 3 to about 200 nucleotides, about 3 to about 50 nucleotides, about 3 to about 25 nucleotides, about 5 to about 25 nucleotides, about 5 to about 15, or about 5 to about 10 nucleotides.

In preferred embodiments, the nucleic acid of the second component component has a length of about 3 to about 50 nucleotides, about 5 to about 25 nucleotides, about 5 to about 15, or about 5 to about 10 nucleotides.

In particularly preferred embodiments, the nucleic acid of the second component component has a length of about 5 to about 15 nucleotides.

In various specific embodiments, the nucleic acid of the second component has a length of 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides.

In preferred specific embodiments, the nucleic acid of the second component has a length of 6 nucleotides, 7 nucleotides, 8 nucleotides, 9 nucleotides, 10 nucleotides, 11 nucleotides, or 12 nucleotides. Preferably, the nucleic acid of the second component has a length of 9 nucleotides.

In preferred embodiments, the nucleic acid of the second component is a single stranded oligonucleotide. In particularly preferred embodiments, the nucleic acid of the second component is a single stranded RNA oligonucleotide.

An RNA oligonucleotide in the context of the invention comprises RNA nucleotides and, preferably, at least one chemically modified RNA nucleotide. An RNA oligonucleotide is a short RNA molecule having a length that typically does not exceed 200 nucleotides. Typically, RNA oligonucleotides are chemically synthesized using building blocks, protected phosphoramidites of natural or chemically modified nucleosides.

The nucleoside residues of an oligonucleotide can be coupled to each other by any of the numerous known internucleoside linkages. Such internucleoside linkages include, without limitation, phosphodiester, phosphorothioate, phosphorodithioate, alkylphosphonate, alkylphosphonothioate, phosphotriester, phosphoramidate, siloxane, carbonate, carboalkoxy, acetamidate, carbamate, morpholino, borano, thioether, bridged phosphoramidate, bridged methylene phosphonate, bridged phosphorothioate, and sulfone internucleoside linkages. The term “oligonucleotide” also encompasses polynucleosides having one or more stereospecific internucleoside linkage (e.g., (Rp)- or (5)-phosphorothioate, alkylphosphonate, or phosphotriester linkages). Preferred in the context of the invention is phosphodiester linkage.

The oligonucleotide chain assembly proceeds in the direction from 3′- to 5-terminus by following a routine procedure referred to as a “synthetic cycle”. Completion of a single synthetic cycle results in the addition of one nucleotide residue to the growing chain. Accordingly, in the context of the invention, the nucleic acid of the second component is a single stranded synthetic RNA oligonucleotide.

In some embodiments, the antagonist of the second component, preferably the nucleic acid comprises two or more different nucleic acids e.g. oligonucleotides as defined herein linked to a nucleotide or a non-nucleotide linker, herein referred to as being “branched.” In some embodiments, the antagonist of the second component, preferably the nucleic acid comprises two or more different nucleic acids e.g. oligonucleotides as defined herein, wherein said two or more nucleic acids e.g. oligonucleotides are non- covalently linked, such as by electrostatic interactions, hydrophobic interactions, T-stacking interactions, hydrogen bonding and combinations thereof. Non-limiting examples of such non-covalent linkage includes Watson-Crick base pairing, Hoogsteen base pairing, and base stacking.

In some embodiments, the antagonist of the second component, preferably the nucleic acid comprises a motif selected from CpG, C*pG, C*pG* and CpG*, wherein C is 2′- deoxycytidine, G is 2′-deoxy guanosine, C* is 2′-deoxythymidine, I-(2′-deoxy-B-D- ribofuranosyl)-2-oxo-7-deaza-8-methyl-purine, 5-Me-dC, 2′-dideoxy-5-halocytosine, 2′-dideoxy-5-nitrocytosine, arabinocytidine, 2′-deoxy-2′-substituted arabinocytidine, 2′-O-substituted arabinocytidine, 2′-deoxy-5-hydroxycytidine, 2′-deoxy-N4-alkyl- cytidine, 2′-deoxy-4-thiouridine, 2′-O-substituted ribonucleotides (including, but not limited to, 2′-O-Me-5-Me-C, 2′-O-(2-methoxyethyl)-ribonucelotides or 2′-O-Me- ribonucleotides) or other cytosine nucleotide derivative, G* is 2′-deoxy-7- deazaguanosine, 2′-deoxy-6-thioguanosine, arabinoguanosine, 2′-deoxy-2′ substituted- arabinoguanosine, 2′-O-substituted-arabinoguanosine, 2′-deoxyinosine, 2′-O-substituted ribonucleotides (including, but not limited to, 2′-O-(2-methoxyethyl)- ribonucelotides; or 2′-O-Me-ribonucleotides) or other guanine nucleotide derivative, and p is an internucleoside linkage selected from the group consisting of phosphodiester, phosphorothioate, and phosphorodithioate.

In some embodiments, the antagonist of the second component, preferably the nucleic acid, comprises a 7-deazaguanosine (c7G) and at least one UpG-containing motif.

In the art it has been shown that bacterial tRNA^{Tyr} sequence fragments may function as TLR antagonists (Schmitt et al 2017. RNA 23:1344-135). Accordingly, in embodiments, the nucleic acid of the second component comprises or consists of a nucleic acid sequence derived from a bacterial tRNA sequence. Preferably, the nucleic acid sequence is or is derived from a bacterial tRNA^{Tyr} sequence.

In embodiments, the nucleic acid of the second component comprises or consists of a nucleic acid sequence derived from a bacterial tRNA^{Tyr} sequence, wherein the nucleic acid sequence is or is derived from the D-Loop of tRNA^{Tyr}. In preferred embodiments, the nucleic acid sequence is or is derived from the D-Loop of tRNA^{Tyr} of Escherichia coli.

In preferred embodiments, the nucleic acid of the second component is an RNA oligonucleotide, that is a fragment of the D-Loop of tRNA^{Tyr} of Escherichia coli, wherein the fragment has a length of about 5 to about 15 nucleotides, wherein the nucleic acid sequence comprises at least one 2′-O-methylated RNA nucleotide, preferably at least one M-X-Y motif, optionally wherein the RNA Oligonucleotide is devoid of a triphosphate 5′ terminus, optionally wherein the M-X-Y motif is not positioned at the 3′ terminus of the RNA oligonucleotide.

In embodiments of the invention, the nucleic acid of the second component, preferably the oligonucleotide, comprises or consists of a nucleic acid sequence identical or at least 70%, 80%, 85%, 86%, 87%, 88%, 89%, 90%, 91%, 92%, 93%, 94%, 95%, 96%, 97%, 98%, or 99% identical to a nucleic acid sequence selected from the group consisting of SEQ ID NOs: 85-165, or fragments of any of these sequences. Additional information regarding each of these suitable nucleic acid sequences may also be derived from the sequence listing, in particular from the details provided therein under identifier <223>.

In preferred embodiments of the invention, the nucleic acid of the second component, preferably the oligonucleotide, comprises or consists of a nucleic acid sequence identical or at least 70%, 80%, 85%, 86%, 87%, 88%, 89%, 90%, 91%, 92%, 93%, 94%, 95%, 96%, 97%, 98%, or 99% identical to a nucleic acid sequence selected from the group consisting of SEQ ID NOs: 85-100, 149-165 or fragments of any of these sequences.

In more preferred embodiments of the invention, the nucleic acid of the second component, preferably the oligonucleotide, comprises or consists of a nucleic acid sequence identical or at least 70%, 80%, 85%, 86%, 87%, 88%, 89%, 90%, 91%, 92%, 93%, 94%, 95%, 96%, 97%, 98%, or 99% identical to a nucleic acid sequence selected from the group consisting of SEQ ID NOs: 85-87, 149-165, or provided in Table B, rows 1-20, or fragments of any of these sequences.

Particularly preferred in that context is a nucleic acid sequence identical or at least 70%, 80%, 85%, 86%, 87%, 88%, 89%, 90%, 91%, 92%, 93%, 94%, 95%, 96%, 97%, 98%, or 99% identical to a nucleic acid sequence according SEQ ID NO: 85, or provided in Table B, row 1, or fragments of any of these sequences.

In the Table below (Table B), suitable nucleic acid sequences of the second component are provided, wherein modified nucleotides (e.g. Gm) are indicated; preferably, the sequences provided in Table B are RNA oligonucleotides. Particularly preferred is the RNA oligonucleotide 5′-GAG CGmG CCA-3′ (see Table B, row 1), wherein position 5 of said RNA oligonucleotide is a 2′-O-methylated guanosine (Gm). Additional information regarding each of these suitable nucleic acid sequences may also be derived from the sequence listing, in particular from the details provided therein under identifier <223>.

TABLE B
Preferred oligonucleotide antagonists of the invention:

| | | SEQ ID |
|---|---|---|
| Row | Sequence | NO: |
| 1 | GAGC [symbol] GCCA | 85 |
| 2 | AGC [symbol] GCC | 86 |
| 3 | GC [symbol] GC | 87 |
| 4 | GAGA [symbol] GCCA | 149 |
| 5 | GAGG [symbol] GCCA | 150 |
| 6 | GAGU [symbol] GCCA | 151 |
| 7 | GAGC [symbol] GCCA | 152 |
| 8 | GAGCUmGCCA | 153 |
| 9 | GAGCGmACCA | 154 |
| 10 | GAGCGmCCCA | 155 |
| 11 | GAGCGmUCCA | 156 |
| 12 | GAGCGmGACA | 157 |
| 13 | GAGCGmGGCA | 158 |
| 14 | GAGCGmGUCA | 159 |
| 15 | GAGCGGmCCA | 160 |
| 16 | GAGCGGGmCA | 161 |
| 17 | GAGCGGCGmA | 162 |
| 18 | GAGGmGGCCA | 163 |
| 19 | GAGmCGGCCA | 164 |
| 20 | GGmGCGGCCA | 165 |
| 21 | G*A*G*C*Gm*G*C*C*A | 187 |
| 22 | GCGmGCCAAA | 188 |
| 23 | G*C*Gm*G*C*C*A*A*A | 189 |
| 24 | CCGAGCGmGC | 190 |
| 25 | GA6mGCGmGCCA6m | 191 |
| 26 | GAGC4AcGmGC4AcC4AcA | 192 |
| 27 | dT*dC*dC*dT*dG*dG*dC*dG*dG*dG*dG*dA*dA*dG*dT | 193 |
| 28 | dT*dA*dA*dT*dG*dG*dC*dG*dG*dG*dG*dA*dA*dG*dT | 194 |
| 29 | dT*dC*dC*dT*dG*dA*dG*dC*dT*dT*dG*dA*dA*dG*dT | 195 |
| 30 | dT*dC*dC*dT*dA*dA*dC*dA*dA*dA*dA*dA*dA*dA*dT | 196 |
| 31 | T*G*C*T*C*C*T*G*G*A*G*G*G*G*T*T*G*T | 197 |
| 32 | T*G*C*T*T*G*C*A*A*G*C*T*T*G*C*A*A*G*C*A | 198 |
| 33 | T*C*C*T*G*G*C*mE*G*G*G*A*A*G*T | 199 |
| 34 | CTATCTGmAmCGTTCTCTGT | 200 |
| 35 | mUmUmUmUmUmUmUmUmUmUmUmUmUmUmUmUmUmUmUmUmU | 207 |
| 36 | GAmUmUAmUGmUCCGGmUmUAmUGmUAUU | 107 |
| 37 | Am*Um*A*Am*Um*U*U*U*Um*Um*G*G*U*Am*Um*U*U | 201 |
| 38 | G*A*Um*A*C*U*U*A*C*C*U*G | 202 |
| 39 | UmGmCmUmCmCmUmGmGmAmGmGmGmGmUmUmGmU | 203 |
| 40 | UUGAUGmUGmUUUAGUCGCUAUU | 204 |
| 41 | GGU GGG GUU CCC GAG CGmG CCA AAG GGA | 205 |
| 42 | GGUmCUmACUmUmUm | 206 |

*Phosphorothioate (PTO) backbone, d = deoxy, A6m = N6-methyladenosine, 4Ac = N4-acetylcytosine, mE = 7-deaza-2′-O-methyl-guanine

In other embodiments of the invention, the nucleic acid of the second component, preferably the oligonucleotide may be selected from IRS-954 (DV-1079), IRO-5, IRS 2088, IRS 869, INH-ODN-2114, INH-ODN 4024, INH-ODN 4084-F, IRS-661, IRS-954, INH-ODN-24888, IHN-ODN 2088, ODN 20958, IHN-ODN-21595, IHN-ODN-20844, IHN-ODN-24991, IHN-ODN-105870, IHN-ODN-105871, ODN A151, G-ODN, ODN INH-1, ODN INH-18, ODN 4084-F, INH-4, INH-13, (pS-) ST-ODN, INH-ODN 21 14, CMZ 203-84, CMZ 203-85, CMZ 203-88, CMZ 203-88-1, CMZ 203-91, ODN 4084, ODN INH-47, CpG-52364 (quinazoline derivate from Coley Pharmaceutical), IMO-3100, IMO-8400, IMO-8503 (inhibitory RNA/DNA hybrid oligonucleotide), ODN 2087, ODN 20959, SM934, IMO-4200, IMO-9200, DV-1179, VTX-763, TMX-302, TMX-306 and further oligonucleotides disclosed by Schmitt et al. (Schmitt et al 2017. RNA 23:1344-135.), Robbins et al. (Robbins et al 2007. Molecular therapy Vol 15 No 9, 1663-1669.), WO2008017473 (especially table 2 and table 6, SEQ ID NO: 195-201), WO2009141146 (SEQ ID Nos: 4-56), WO2010105819, US2009087388 (table 4 and table 6), WO2017136399 (table 4) and WO2008033432 (table 1-5 and table 8).

In further specific embodiments, the nucleic acid of the second component, preferably the oligonucleotide, is or is derived from published PCT application WO2009055076, in particular from claims 44 to 45 of WO2009055076. The disclosure of WO2009055076, in particular disclosure relating to claims 44 to 45 of WO2009055076 herewith incorporated by reference.

First Component: Therapeutic RNA

In the following, advantageous embodiments and features of the at least one therapeutic RNA of the first component are described. Notably, all described embodiments and features of said therapeutic RNA that are described in the context of the inventive combination (first aspect) are likewise applicable to the therapeutic RNA of the pharmaceutical composition (second aspect), or the kit or kit of parts (third aspect), and to further aspects of the invention.

In various embodiments, the at least one therapeutic RNA of the first component is selected from a coding RNA, a non-coding RNA, a circular RNA (circRNA), an RNA oligonucleotide, a small interfering RNA (siRNA), a small hairpin RNA (shRNA), an antisense RNA (asRNA), a CRISPR/Cas9 guide RNAs, an mRNA, a riboswitch, an immunostimulating RNA (isRNA), a ribozyme, an RNA aptamer, a ribosomal RNA (rRNA), a transfer RNA (tRNA), a viral RNA (vRNA), a retroviral RNA, a small nuclear RNA (snRNA), a self-replicating RNA, a replicon RNA, a small nucleolar RNA (snoRNA), a microRNA (miRNA), and a Piwi-interacting RNA (piRNA).

The term “RNA” will be recognized and understood by the person of ordinary skill in the art, and is e.g. intended to be a ribonucleic acid molecule, i.e. a polymer consisting of nucleotides. These nucleotides are usually adenosine-monophosphate, uridine-monophosphate, guanosine-monophosphate and cytidine-monophosphate monomers which are connected to each other along a so-called backbone. The backbone is typically formed by phosphodiester bonds between the sugar, i.e. ribose, of a first monomer and a phosphate moiety of a second, adjacent monomer. The specific succession of monomers is called the RNA-sequence.

The term “therapeutic RNA” relates to any RNA, in particular any RNA as defined above, providing a therapeutic modality. The term “therapeutic” in that context has to be understood as “providing a therapeutic function” or as “being suitable for therapy or administration”. However, “therapeutic” in that context should not at all to be understood as being limited to a certain therapeutic modality. Examples for therapeutic modalities may be the provision of a coding sequence (via said therapeutic RNA) that encodes for a peptide or protein (wherein said peptide or protein has a certain therapeutic function, e.g. an antigen for a vaccine, or an enzyme for protein replacement therapies). A further therapeutic modality may be genetic engineering, wherein the RNA provides or orchestrates factors to e.g. manipulate DNA and or RNA. Typically, the term “therapeutic RNA” does not include natural RNA extracts or RNA preparations (e.g. obtained from bacteria, or obtained from plants) that are not suitable for administration to a subject (e.g. animal, human). For being suitable for a therapeutic purpose, the RNA of the invention may be an artificial, non-natural RNA.

Accordingly, in preferred embodiments, the at least one therapeutic RNA of the first component is an artificial RNA.

The term “artificial RNA” as used herein is intended to refer to an RNA that does not occur naturally. In other words, an artificial RNA may be understood as a non-natural RNA molecule. Such RNA molecules may be non-natural due to their individual sequence (e.g. G/C content modified coding sequence, UTRs) and/or due to other modifications, e.g. structural modifications of modified nucleotides. Artificial RNA may be designed and/or generated by genetic engineering to correspond to a desired artificial sequence of nucleotides. In this context an artificial RNA is a sequence that may not occur naturally, i.e. it differs from the wild type sequence by at least one nucleotide/modification.

In embodiments, the at least one therapeutic RNA of the first component is a non-coding RNA preferably selected from RNA oligonucleotide, a small interfering RNA (siRNA), a small hairpin RNA (shRNA), an antisense RNA (asRNA), a CRISPR/Cas9 guide RNAs, a riboswitch, a ribozyme, an RNA aptamer, a ribosomal RNA (rRNA), a transfer RNA (tRNA), a small nuclear RNA (snRNA), a small nucleolar RNA (snoRNA), a microRNA (miRNA), and a Piwi-interacting RNA (piRNA).

In preferred embodiments, the least one therapeutic RNA of the first component is a non-coding RNA, preferably a CRISPR/Cas9 guide RNA or a small interfering RNA (siRNA).

As used herein, the term “guide RNA” (gRNA) relates to any RNA molecule capable of targeting a CRISPR-associated protein/CRISPR-associated endonuclease to a target DNA sequence of interest. In the context of the invention, the term guide RNA has to be understood in its broadest sense, and may comprise two-molecule gRNAs (“tracrRNA/crRNA”) comprising crRNA (“CRISPR RNA” or “targeter-RNA” or “crRNA” or “crRNA repeat”) and a corresponding tracrRNA (“trans-acting CRISPR RNA” or “activator-RNA” or “tracrRNA”) molecule, or single-molecule gRNAs. A “sgRNA” typically comprises a crRNA connected at its 3′ end to the 5′ end of a tracrRNA through a “loop” sequence. In the context of the invention, a guide RNA may be provided by the at least one therapeutic RNA of the inventive combination/composition.

In preferred embodiments, the at least one therapeutic RNA of the first component is a coding RNA. Most preferably, said coding RNA may be selected from an mRNA, a (coding) self-replicating RNA, a (coding) circular RNA, a (coding) viral RNA, or a (coding) replicon RNA.

A coding RNA can be any type of RNA construct (for example a double stranded RNA, a single stranded RNA, a circular double stranded RNA, or a circular single stranded RNA) characterized in that said coding RNA comprises at least one sequence (cds) that is translated into at least one amino-acid sequence (upon administration to e.g a cell).

The terms “coding sequence”, “coding region”, or “cds” as used herein will be recognized and understood by the person of ordinary skill in the art, and are e.g. intended to refer to a sequence of several nucleotides which may be translated into a peptide or protein. In the context of the present invention a cds is preferably an RNA sequence, consisting of a number of nucleotide triplets, starting with a start codon and preferably terminating with one stop codon. In embodiments, the cds of the RNA may terminate with one or two or more stop codons. The first stop codon of the two or more stop codons may be TGA or UGA and the second stop codon of the two or more stop codons may be selected from TAA, TGA, TAG, UAA, UGA or UAG.

In embodiments, the at least one therapeutic RNA of the first component is a circular RNA. As used herein, “circular RNA” or “circRNAs” have to be understood as a circular polynucleotide constructs that may encode at least one peptide or protein. Accordingly, in preferred embodiments, said circRNA comprises at least one cds encoding at least one peptide or protein as defined herein. circRNA can be synthetized using various methods provided in the art, including e.g. methods as provided in U.S. Pat. Nos. 6,210,931, 5,773,244, WO1992/001813, WO2015/034925 and WO2016/011222, the disclosure relating to circRNA synthesis incorporated herewith by reference.

In embodiments, the at least one therapeutic RNA of the first component is a replicon RNA. The term “replicon RNA” will be recognized and understood by the person of ordinary skill in the art, and is e.g. intended to be an optimized self-replicating RNA. Such constructs may include replicase elements derived from e.g. alphaviruses (e.g. SFV, SIN, VEE, or RRV) and the substitution of the structural virus proteins with the nucleic acid of interest, and a coding sequence. Alternatively, the replicase may be provided on an independent RNA construct. Downstream of the replicase may be a sub-genomic promoter that controls replication of the replicon RNA.

In particularly preferred embodiments, the at least one therapeutic RNA of the first component is a messenger RNA (mRNA). A typical mRNA (messenger RNA) in the context of the invention provides the coding sequence that is translated into an amino-acid sequence of a peptide or protein after e.g. in vivo administration to a cell.

In preferred embodiments, the at least one therapeutic RNA of the first component, in particular the coding RNA or the mRNA, is an in vitro transcribed RNA. Suitably in that context, the therapeutic RNA is an in vitro transcribed coding RNA or in vitro transcribed mRNA.

An in vitro transcribed RNA has to be understood as an RNA that is obtained by RNA in vitro transcription.

The terms “RNA in vitro transcription” or “in vitro transcription” relate to a process wherein RNA is synthesized in a cell-free system (in vitro). RNA may be obtained by DNA-dependent RNA in vitro transcription of an appropriate DNA template, which is a linearized plasmid DNA template or a PCR-amplified DNA template. The promoter for controlling RNA in vitro transcription can be any promoter for any DNA-dependent RNA polymerase. Particular examples of DNA-dependent RNA polymerases are the T7, T3, SP6, or Syn5 RNA polymerases. In a preferred embodiment the DNA template is linearized with a suitable restriction enzyme, before it is subjected to RNA in vitro transcription.

Reagents typically used in RNA in vitro transcription include: a DNA template (linearized plasmid DNA or PCR product) with a promoter sequence that has a high binding affinity for its respective RNA polymerase such as bacteriophage-encoded RNA polymerases (T7, T3, SP6, or Syn5); ribonucleotide triphosphates (NTPs) for the four bases (adenine, cytosine, guanine and uracil); optionally, a cap analogue as defined; optionally, further modified nucleotides as defined herein; a DNA-dependent RNA polymerase capable of binding to the promoter sequence within the DNA template (e.g. T7, T3, SP6, or Syn5 RNA polymerase); optionally, a ribonuclease (RNase) inhibitor to inactivate any potentially contaminating RNase; optionally, a pyrophosphatase to degrade pyrophosphate; MgCl2, which supplies Mg2+ ions as a co-factor for the polymerase; a buffer (TRIS or HEPES) to maintain a suitable pH value, which can also contain antioxidants (e.g. DTT), and/or polyamines such as spermidine at optimal concentrations, e.g. a buffer system comprising TRIS-Citrate as disclosed in WO2017/109161.

Accordingly, in preferred embodiments, the at least one therapeutic RNA of the first component, in particular the coding RNA or the mRNA, is an in vitro transcribed RNA, wherein the in vitro transcribed RNA is obtainable by RNA in vitro transcription using a sequence optimized nucleotide mixture.

In that context, the nucleotide mixture used in RNA in vitro transcription may additionally contain modified nucleotides as defined below. In preferred embodiments, the nucleotide mixture (i.e. the fraction of each nucleotide in the mixture) used for RNA in vitro transcription reactions is essentially optimized for the given RNA sequence (optimized NTP mix), preferably as described WO2015/188933. RNA obtained by a process using an optimized NTP mix is characterized by reduced immune stimulatory properties, which is preferred in the context of the invention.

In preferred embodiments, the at least one therapeutic RNA of the first component, in particular the coding RNA or the mRNA, is a purified RNA (e.g. a purified, in-vitro transcribed mRNA).

The term “purified RNA” as used herein has to be understood as therapeutic RNA which has a higher purity after certain purification steps (e.g. (RP)-HPLC, TFF, Oligo d(T) purification, precipitation steps) than the starting material (e.g. in vitro transcribed RNA or synthetic RNA). Typical impurities essentially not present in purified RNA comprise peptides or proteins (e.g. enzymes derived from RNA in vitro transcription, e.g. RNA polymerases, RNases, pyrophosphatase, restriction endonuclease, DNase), spermidine, BSA, abortive RNA sequences, RNA fragments (short double stranded RNA fragments, abortive sequences etc.), free nucleotides (modified nucleotides, conventional NTPs, cap analogue), template DNA fragments, buffer components (HEPES, TRIS, MgCl2) etc. Other potential impurities that may be derived from e.g. fermentation procedures comprise bacterial impurities (bioburden, bacterial DNA) or impurities derived from purification procedures (organic solvents etc.). Accordingly, it is desirable in this regard for the “degree of RNA purity” to be as close as possible to 100%. It is also desirable for the degree of RNA purity that the amount of full-length RNA transcripts is as close as possible to 100%. Accordingly “purified RNA” as used herein has a degree of purity of more than 70%, 80%, 85%, very particularly 90%, 95%, and most favourably 99% or more. Moreover, “purified RNA” as used herein may additionally, or alternatively, have an amount of full-length RNA of more than 70%, 80%, 85%, very particularly 90%, 95%, and most favourably 99% or more. Such purified RNA as defined herein is characterized by reduced immune stimulatory properties (as compared to non-purified RNA), which is particularly preferred in the context of the invention.

The degree of purity or the amount of full-length RNA may for example be determined by an analytical HPLC, wherein the percentages provided above correspond to the ratio between the area of the peak for the desired RNA and the total area of all peaks in the chromatogram. Alternatively, the degree of purity may be determined by other means for example by an analytical agarose gel electrophoresis or capillary gel electrophoresis.

In the context of the invention, in particular for medical applications, it may be required to provide pharmaceutical-grade RNA. In a particularly preferred embodiment, RNA manufacturing is performed under current good manufacturing practice (GMP), implementing various quality control steps on DNA and RNA level, preferably following a procedure as described in WO2016/180430. The obtained RNA products are preferably purified using RP-HPLC (as described in WO2008/077592) and/or tangential flow filtration (as described in WO2016/193206). Accordingly, in preferred embodiments, the at least one therapeutic RNA of the first component, in particular the coding RNA or the mRNA, is GMP-grade RNA or pharmaceutical-grade RNA.

In preferred embodiments, the at least one therapeutic RNA of the first component, in particular the coding RNA or the mRNA, is a purified RNA (e.g. a purified, in-vitro transcribed mRNA), wherein the purified RNA is purified by RP-HPLC and/or TFF and/or Oligo d(T) purification. Preferably the purified RNA is a (RP)-HPLC purified RNA.

It has to be emphasised that “purified RNA” as defined herein or “pharmaceutical-grade RNA” as defined herein may have superior stability characteristics (in vitro, in vivo) and improved efficiency (e.g. better translatability of the RNA in vivo) and are therefore particularly suitable for any medical purpose. Further, such RNA is characterized by reduced immune stimulatory properties (as compared to non-purified RNA), which is preferred in the context of the invention.

In specific embodiments, the at least one therapeutic RNA of the first component, in particular the coding RNA or the mRNA, is an in vitro transcribed RNA, purified RNA, pharmaceutical grade RNA. Such an RNA is characterized by reduced immune stimulatory properties (as compared to e.g. non-purified in vitro transcribed RNA) and is therefore particularly suitable in the context of the invention.

In preferred embodiments, the at least one therapeutic RNA of the first component, e.g. the coding RNA or the mRNA, comprises at least one coding sequence (cds) encoding at least one peptide or protein.

Advantageously, the expression of the encoded at least one peptide or protein of the coding RNA or the mRNA is increased or prolonged by the combination with the at least one antagonist of at least one RNA sensing receptor of the second component upon administration into cells, a tissue or an organism compared to the expression of the encoded at least one peptide or protein of the coding RNA or the mRNA without combination with the at least one antagonist of at least one RNA sensing pattern recognition receptor of the second component.

Accordingly, administration of the combination (that is, administration of the first and the second component) to a cell, tissue, or organism results in an increased or prolonged peptide/protein expression as compared to administration of the corresponding first component/the therapeutic RNA only.

Methods to evaluate the expression (that is, protein expression) of the therapeutic RNA in specific cells/organs/tissues, and methods to determine the duration of expression are well known in the art for the skilled artisan. For example, protein expression can be determined using antibody-based detection methods (western blots, FACS) or quantitative mass spectrometry. Exemplary methods are provided in the examples section. Typically, the expression of the therapeutic RNA in combination with the second component is compared with the expression of the therapeutic RNA alone (or with the first component alone), that is, without the (additional) administration of the second component. The same conditions (e.g. the same cell lines, same organism, same application route, the same detection method, the same amount of therapeutic RNA, the same RNA sequence) have to be used (if feasible) to allow a valid comparison. The person of skill in the art understands how to perform a comparison of the inventive combination and a respective control RNA (e.g. therapeutic RNA only or first component only).

“Increased protein expression” of the inventive combination has to be understood as percentage increase of expression compared to a corresponding control (first component only or therapeutic RNA only) which can be determined by various well-established expression assays (e.g. antibody-based detection methods) as described above.

Accordingly, administration of the combination (that is, administration of the first and the second component) to a cell, tissue, or organism results in an increased expression as compared to administration of the corresponding first component/the therapeutic RNA only, wherein the percentage increase in expression in said cell, tissue, or organism is at least 10%, 20%, 30%, 40%, 50%, 60%, 70%, 80%, 90%, 100%, 200%, 300%, 400%, 500% or even more.

“Prolonged protein expression” of the inventive combination has to be understood as the additional duration of protein expression wherein expression of the inventive combination is still detectable in comparison to a corresponding control (first component only or therapeutic RNA only) which can be determined by various well-established expression assays (e.g. antibody-based detection methods) as described above.

Accordingly, administration of the combination (that is, administration of the first and the second component) to a cell, tissue, or organism results in a prolonged protein expression compared to administration of the corresponding first component/the therapeutic RNA only, wherein the additional duration of protein expression in said cell, tissue, or organism is at least 5h, 10h, 20h, 25h, 30h, 35h, 40h, 45h, 50h, 55h, 60h, 65h, 70h, 75h, 80h, 85h, 90h, 95h, or 10h or even longer.

In particularly preferred embodiments, the expression of the encoded at least one peptide or protein of the coding RNA or the mRNA is increased or prolonged by the combination with the at least one antagonist of at least one RNA sensing receptor of the second component upon administration into cells, a tissue or an organism compared to the expression of the encoded at least one peptide or protein of the coding RNA or the mRNA without combination with the at least one antagonist of at least one RNA sensing pattern recognition receptor of the second component, whereas, at the same time administration of the combination of the at least one coding RNA or the mRNA and the at least one antagonist of at least one RNA sensing pattern recognition receptor of the second component leads to a reduced innate immune response compared to administration of the at least one coding RNA or the mRNA of the first component without combination with the at least one antagonist of at least one RNA sensing pattern recognition receptor of the second component.

In preferred embodiments, the cds of the coding RNA or mRNA, encodes at least one peptide or protein, wherein said at least one peptide or protein is or is derived from a therapeutic peptide or protein.

In various embodiments, the length of the encoded peptide or protein, e.g. the therapeutic peptide or protein, may be at least or greater than about 20, 50, 100, 150, 200, 300, 400, 500, 600, 700, 800, 900, 1000, or 1500 amino acids.

In embodiments, the at least one therapeutic peptide or protein is or is derived from an antibody, an intrabody, a receptor, a receptor agonist, a receptor antagonist, a binding protein, a CRISPR-associated endonuclease, a chaperone, a transporter protein, an ion channel, a membrane protein, a secreted protein, a transcription factor, an enzyme, a peptide or protein hormone, a growth factor, a structural protein, a cytoplasmic protein, a cytoskeletal protein, a viral antigen, a bacterial antigen, a protozoan antigen, an allergen, a tumor antigen, or fragments, variants, or combinations of any of these.

In some embodiments the antibodies coded by the RNA or mRNA according to the invention can be chosen from all antibodies, e.g. from all antibodies which are generated by recombinant methods or are naturally occurring and are known to a person skilled in the art from the prior art, in particular antibodies which are (can be) employed for therapeutic purposes or for diagnostic or for research purposes or have been found with particular diseases, e.g. cancer diseases, infectious diseases etc as also described in WO2008083949 included herewith by reference.

In the context of the present invention, antibodies which are coded by an RNA or mRNA according to the invention typically include all antibodies which are known to a person skilled in the art, e.g. naturally occurring antibodies or antibodies generated in a host organism by immunization, antibodies prepared by recombinant methods which have been isolated and identified from naturally occurring antibodies or antibodies generated in a host organism by (conventional) immunization or have been generated with the aid of molecular biology methods, as well as chimeric anti-bodies, human antibodies, humanized antibodies, bispecific antibodies, intrabodies, i.e. antibodies expressed in cells and possibly localized in particular cell compartments, and fragments of the abovementioned antibodies. Insofar, the term antibody is to be understood in its broadest meaning. In this context, antibodies in general typically comprise a light chain and a heavy chain, both of which have variable and constant domains.

According to embodiments, the cds of the at least one therapeutic RNA as defined herein, encodes at least one (therapeutic) peptide or protein as defined above, and additionally at least one further heterologous peptide or protein element.

Suitably, the at least one further heterologous peptide or protein element may be selected from secretory signal peptides, transmembrane elements, multimerization domains, VLP forming sequence, a nuclear localization signal (NLS), peptide linker elements, self-cleaving peptides, immunologic adjuvant sequences or dendritic cell targeting sequences.

According to preferred embodiments, the therapeutic RNA of the first component comprises at least one cds, wherein the cds encodes at least one peptide or protein as specified herein. In that context, any cds encoding at least one peptide or protein may be understood as suitable cds and may therefore be comprised in the therapeutic RNA.

In embodiments, the length the cds may be at least or greater than about 50, 60, 70, 80, 90, 100, 150, 200, 250, 300, 350, 400, 450, 500, 600, 700, 800, 900, 1000, 1200, 1400, 1600, 1800, 2000, 2500, 3000, 3500, 4000, 5000, or 6000 nucleotides. In embodiments, the length of the cds may be in a range of from about 300 to about 2000 nucleotides.

In preferred embodiments, the therapeutic RNA of the first component is a modified and/or stabilized RNA, preferably a modified and/or stabilized coding RNA or a modified and/or stabilized mRNA.

The therapeutic RNA of the first component may thus be provided as a “stabilized artificial RNA” that is to say an RNA showing improved resistance to in vivo degradation and/or an RNA showing improved stability in vivo, and/or an RNA showing improved translatability in vivo.

In the following, modifications are described that are suitably to “stabilize” the therapeutic RNA of the first component.

In preferred embodiments, the at least one cds of the therapeutic RNA of the first component is a codon modified cds, wherein the amino acid sequence encoded by the at least one codon modified cds is preferably not being modified compared to the amino acid sequence encoded by the corresponding wild type cds.

The term “codon modified coding sequence” relates to coding sequences that differ in at least one codon (triplets of nucleotides coding for one amino acid) compared to the corresponding wild type cds. A codon modified cds in the context of the invention shows improved resistance to in vivo degradation and/or improved stability in vivo, and/or improved translatability in vivo. Codon modifications make use of the degeneracy of the genetic code as multiple codons encoding the same amino acid can be used interchangeably to optimize/modify a coding sequence (Table 1).

In particularly preferred embodiments, the at least one cds of the therapeutic RNA of the first component is a codon modified cds, wherein the codon modified cds is selected from C maximized cds, CAI maximized cds, human codon usage adapted cds, G/C content modified cds, and G/C optimized cds, or any combination thereof.

In preferred embodiments, the therapeutic RNA of the first component may be modified, wherein the C content of the at least one cds may be increased, preferably maximized, compared to the C content of the corresponding wild type cds (herein referred to as “C maximized coding sequence”). The amino acid sequence encoded by the C maximized cds is preferably not modified as compared to the amino acid sequence encoded by the respective wild type nucleic acid cds. The generation of a C maximized nucleic acid sequences may be carried out using a method according to WO2015/062738, the disclosure of WO2015/062738 included herewith by reference.

In embodiments, the therapeutic RNA of the first component may be modified, wherein the G/C content of the at least one cds may be modified compared to the G/C content of the corresponding wild type cds (herein referred to as “G/C content modified coding sequence”). In this context, the terms “G/C optimization” or “G/C content modification” relate to RNA that comprises a modified, preferably an increased number of guanosine and/or cytosine nucleotides as compared to the corresponding wild type RNA. Such an increased number may be generated by substitution of codons containing A or T nucleotides by codons containing G or C nucleotides.

Advantageously, RNA sequences having an increased G/C content are more stable (which may lead to an increased translation in vivo) than the corresponding wild type sequences or than sequences having an increased A/U content. The amino acid sequence encoded by the G/C content modified cds is preferably not modified as compared to the amino acid sequence encoded by the respective wild type sequence. Preferably, the G/C content of the at least one cds is increased by at least 10%, 20%, 30%, preferably by at least 40% compared to the G/C content of the cds of the corresponding wild type sequence.

In preferred embodiments, the therapeutic RNA of the first component may be modified, wherein the G/C content of the at least one cds may be optimized compared to the G/C content of the corresponding wild type cds (herein referred to as “G/C content optimized coding sequence”). “Optimized” in that context refers to a cds wherein the G/C content is preferably increased to essentially the highest possible G/C content. The amino acid sequence encoded by the G/C content optimized cds is preferably not modified as compared to the amino acid sequence encoded by the respective wild type cds. Advantageously, RNA sequences having a G/C content optimized coding sequence are more stable (which may lead to an increased translation in vivo) than the corresponding wild type sequences. The generation of a G/C content optimized coding sequences may be carried out according to WO2002/098443, the disclosure of WO2002/098443 included herewith by reference.

In embodiments, the therapeutic RNA of the first component may be modified, wherein the codons in the at least one cds may be adapted to human codon usage (herein referred to as “human codon usage adapted coding sequence”). Codons encoding the same amino acid occur at different frequencies in a subject, e.g. a human. Accordingly, the cds is preferably modified such that the frequency of codons encoding the same amino acid corresponds to the naturally occurring frequency of that codon according to the human codon usage. E.g., in the case of the amino acid Ala, the wild type cds is preferably adapted in a way that codon “GCC” is used with a frequency of 0.40, codon “GCT” is used with a frequency of 0.28, codon “GCA” is used with a frequency of 0.22 and codon “GCG” is used with a frequency of 0.10 etc. (see Table 1). Accordingly, such a procedure (as exemplified for Ala) is applied for each amino acid encoded by the cds to obtain sequences adapted to human codon usage. Advantageously, RNA sequences having a human codon usage adapted coding sequence may be more stable or show better translatability in vivo, than corresponding wild type sequences.

Table 1: Human codon usage with respective codon frequencies indicated for each amino acid

TABLE 1
Human codon usage with respective codon
frequencies indicated for each amino acid

| Amino acid | codon | frequency |
|---|---|---|
| Ala | GCG | 0.10 |
| Ala | GCA | 0.22 |
| Ala | GCT | 0.28 |
| Ala | GCC* | 0.40 |
| Cys | TGT | 0.42 |
| Cys | TGC* | 0.58 |
| Asp | GAT | 0.44 |
| Asp | GAC* | 0.56 |
| Glu | GAG* | 0.59 |
| Glu | GAA | 0.41 |
| Phe | TTT | 0.43 |
| Phe | TTC* | 0.57 |
| Gly | GGG | 0.23 |
| Gly | GGA | 0.26 |
| Gly | GGT | 0.18 |
| Gly | GGC* | 0.33 |
| His | CAT | 0.41 |
| His | CAC* | 0.59 |
| Ile | ATA | 0.14 |
| Ile | ATT | 0.35 |
| Ile | ATC* | 0.52 |
| Lys | AAG* | 0.60 |
| Lys | AAA | 0.40 |
| Leu | TTG | 0.12 |
| Leu | TTA | 0.06 |
| Leu | CTG* | 0.43 |
| Leu | CTA | 0.07 |
| Leu | CTT | 0.12 |
| Leu | CTC | 0.20 |
| Met | ATG* | 1 |
| Asn | AAT | 0.44 |
| Asn | AAC* | 0.56 |
| Pro | CCG | 0.11 |
| Pro | CCA | 0.27 |
| Pro | CCT | 0.29 |
| Pro | CCC* | 0.33 |
| Gln | CAG* | 0.73 |
| Gln | CAA | 0.27 |
| Arg | AGG | 0.22 |
| Arg | AGA* | 0.21 |
| Arg | CGG | 0.19 |
| Arg | CGA | 0.10 |
| Arg | CGT | 0.09 |
| Arg | CGC | 0.19 |
| Ser | AGT | 0.14 |
| Ser | AGC* | 0.25 |
| Ser | TCG | 0.06 |
| Ser | TCA | 0.15 |
| Ser | TCT | 0.18 |
| Ser | TCC | 0.23 |
| Thr | ACG | 0.12 |
| Thr | ACA | 0.27 |
| Thr | ACT | 0.23 |
| Thr | ACC* | 0.38 |
| Val | GTG* | 0.48 |
| Val | GTA | 0.10 |
| Val | GTT | 0.17 |
| Val | GTC | 0.25 |
| Trp | TGG* | 1 |
| Tyr | TAT | 0.42 |
| Tyr | TAC* | 0.58 |
| Stop | TGA* | 0.61 |
| Stop | TAG | 0.17 |
| Stop | TAA | 0.22 |

*: most frequent human codon for a certain amino acid

In embodiments, the therapeutic RNA of the first component may be modified, wherein the codon adaptation index (CAI) may be increased or preferably maximised in the at least one cds (herein referred to as “CAI maximized coding sequence”). Accordingly, it is preferred that all codons of the wild type nucleic acid sequence that are relatively rare in e.g. a human cell are exchanged for a respective codon that is frequent in the e.g. a human cell, wherein the frequent codon encodes the same amino acid as the relatively rare codon. Suitably, the most frequent codons are used for each encoded amino acid (see Table 1, most frequent human codons are marked with asterisks). Suitably, the RNA comprises at least one cds, wherein the codon adaptation index (CAI) of the at least one cds is at least 0.5, at least 0.8, at least 0.9 or at least 0.95. Most preferably, the codon adaptation index (CAI) of the at least one cds is 1. E.g., in the case of the amino acid Ala, the wild type cds is adapted in a way that the most frequent human codon “GCC” is always used for said amino acid. Accordingly, such a procedure (as exemplified for Ala) is applied for each amino acid encoded by the cds to obtain a CAI maximized cds.

In embodiments, the therapeutic RNA (coding RNA or mRNA) of the first component may be modified by the addition of a 5′-cap structure, which preferably stabilizes the RNA and/or enhances expression of the encoded peptide or protein. A 5′-cap structure is of particular importance in embodiments where the therapeutic RNA is linear, e.g. a linear mRNA or a linear replicon RNA. Accordingly, in preferred embodiments, the therapeutic RNA of the first component, preferably the mRNA, comprises a 5′-cap structure.

In preferred embodiments, the 5′-cap structure is an m7G (m7G(5′)ppp(5′)G), cap0, cap1, cap2, a modified cap0 or a modified cap1 structure.

The term “5′-cap structure” as used herein will be recognized and understood by the person of ordinary skill in the art, and is e.g. intended to refer to a 5′ modified nucleotide, particularly a guanine nucleotide, positioned at the 5′-end of an RNA, e.g. an mRNA. Typically, a 5′-cap structure is connected via a 5′-5′-triphosphate linkage to the RNA. 5′-cap structures suitable in the context of the present invention are cap0 (methylation of the first nucleobase, e.g. m7GpppN), cap1 (additional methylation of the ribose of the adjacent nucleotide of m7GpppN), cap2 (additional methylation of the ribose of the 2nd nucleotide downstream of the m7GpppN), cap3 (additional methylation of the ribose of the 3rd nucleotide downstream of the m7GpppN), cap4 (additional methylation of the ribose of the 4th nucleotide downstream of the m7GpppN), ARCA (anti-reverse cap analogue), modified ARCA (e.g. phosphothioate modified ARCA), inosine, N1-methyl-guanosine, 2′-fluoro-guanosine, 7-deaza-guanosine, 8-oxo-guanosine, 2-amino-guanosine, LNA-guanosine, and 2-azido-guanosine.

A 5′-cap (cap0 or cap1) structure may be formed in chemical RNA synthesis or RNA in vitro transcription (co-transcriptional capping) using cap analogues.

The term “cap analogue” as used herein will be recognized and understood by the person of ordinary skill in the art, and is e.g. intended to refer to a non-polymerizable di-nucleotide or tri-nucleotide that has cap functionality in that it facilitates translation or localization, and/or prevents degradation of a nucleic acid molecule, particularly of an RNA molecule, when incorporated at the 5′-end of the nucleic acid molecule. Non-polymerizable means that the cap analogue will be incorporated only at the 5′-terminus because it does not have a 5′ triphosphate and therefore cannot be extended in the 3′-direction by a template-dependent RNA polymerase. Examples of cap analogues include, but are not limited to any one selected from the group consisting of m7GpppG, m7GpppA, m7GpppC; unmethylated cap analogues (e.g. GpppG); dimethylated cap analogue (e.g. m2,7GpppG), trimethylated cap analogue (e.g. m2,2,7GpppG), dimethylated symmetrical cap analogues (e.g. m7Gpppm7G), or anti reverse cap analogues (e.g. ARCA; m7,2′OmeGpppG, m7,2′dGpppG, m7,3′OmeGpppG, m7,3′dGpppG and their tetraphosphate derivatives). Further cap analogues have been described previously (WO2008/016473, WO2008/157688, WO2009/149253, WO2011/015347, and WO2013/059475). Further suitable cap analogues in that context are described in WO2017/066793, WO2017/066781, WO2017/066791, WO2017/066789, WO2017/053297, WO2017/066782, WO2018/075827 and WO2017/066797, the disclosures referring to cap analogues incorporated herewith by reference. Preferred cap-analogues are the di-nucleotide cap analogues m7G(5′)ppp(5′)G (m7G) or 3′-O-Me-m7G(5′)ppp(5′)G to co-transcriptionally generate cap0 structures.

In embodiments, a modified cap1 structure is generated using tri-nucleotide cap analogue as disclosed in WO2017/053297, WO2017/066793, WO2017/066781, WO2017/066791, WO2017/066789, WO2017/066782, WO2018/075827 and WO2017/066797. In particular, any cap structures derivable from the structure disclosed in claim 1-5 of WO2017/053297 may be suitably used to co-transcriptionally generate a modified cap1 structure.

Further, any cap structures derivable from the structure defined in claim 1 or claim 21 of WO2018075827 may be suitably used to co-transcriptionally generate a modified cap1 structure.

In particularly preferred embodiments, the therapeutic RNA of the first component, preferably the mRNA, comprises a cap1 structure. A cap1 structure may be formed enzymatically or co-transcriptionally (e.g. using m7G(5′)ppp(5′)(2′OMeA)pG, or m7G(5′)ppp(5′)(2′OMeG)pG analogues). A cap1 structure comprising RNA, preferably mRNA has several advantageous features in the context of the invention including an increased translation efficiency and a reduced stimulation of the innate immune system.

In preferred embodiments, the 5′-cap structure may suitably be added co-transcriptionally using tri-nucleotide cap analogue as defined herein in an RNA in vitro transcription reaction as defined herein. It is advantageous that the RNA of the first component comprises a cap1 structure, wherein said cap1 structure is obtainable by co-transcriptional capping.

In preferred embodiments, the cap1 structure of the at least one therapeutic RNA is formed using co-transcriptional capping using tri-nucleotide cap analogues m7G(5′)ppp(5′)(2′OMeA)pG or m7G(5′)ppp(5′)(2′OMeG)pG. A preferred cap1 analogue in that context is m7G(5′)ppp(5′)(2′OMeA)pG.

Without being bound to theory, an advantageous effect of generating cap1 structures using co-transcriptional capping may be explained by an improved capping efficiency compared to enzymatic capping, and/or that enzymatic capping can also generate intermediate cap1 structures (e.g. partial methylation of the 5′ cap and/or partial of the ribose following the 5′ cap).

In other embodiments, the 5′-cap structure is formed via enzymatic capping using capping enzymes (e.g. vaccinia virus capping enzymes and/or cap-dependent 2′-O-methyltransferases) to generate capo or cap1 or cap2 structures. The 5′-cap structure (cap0 or cap1) may be added using immobilized capping enzymes and/or cap-dependent 2′-O-methyltransferases using methods and means disclosed in WO2016/193226.

In preferred embodiments, about 70%, 75%, 80%, 85%, 90%, 95% of the therapeutic RNA (species) of the first component comprises a cap1 structure as determined using a capping assay. In preferred embodiments, less than about 20%, 15%, 10%, 5%, 4%, 3%, 2%, 1% of the therapeutic RNA (species) of the first component does not comprises a cap1 structure as determined using a capping assay. In preferred embodiments, less than about 20%, 15%, 10%, 5%, 4%, 3%, 2%, 1% of the therapeutic RNA (species) of the first component comprises a cap0 structure as determined using a capping assay. In preferred embodiments, less than about 20%, 15%, 10%, 5%, 4%, 3%, 2%, 1% of the coding RNA (species) of the first component comprises a cap1 intermediate structure as determined using a capping assay.

The term “therapeutic RNA species” is not restricted to mean “one single molecule” but is understood to comprise an ensemble of essentially identical RNA therapeutic molecules. The term may preferably relate to a plurality of essentially identical coding RNA molecules, encoding the same amino acid sequence.

For determining the capping degree or the presence of cap1 intermediates, a capping assays as described in published PCT application WO2015101416, in particular, as described in Claims 27 to 46 of published PCT application WO2015101416 can be used. Other capping assays that may be used to determine the capping degree of the therapeutic RNA are described in PCT/EP2018/08667, or published PCT applications WO2014/152673 and WO2014152659.

In preferred embodiments, the therapeutic RNA (coding RNA or mRNA) of the first component comprises a 5′ terminal m7G(5′)ppp(5′)(2′OMeA) cap structure. In such embodiments, the RNA comprises a 5′ terminal m7G cap, and an additional methylation of the ribose of the adjacent nucleotide of m7GpppN, in that case, a 2′ methylated adenosine.

In other preferred embodiments, the therapeutic RNA (coding RNA or mRNA) of the first component comprises an m7G(5′)ppp(5′)(2′OMeG) cap structure. In such embodiments, the RNA comprises a 5′ terminal m7G cap, and an additional methylation of the ribose of the adjacent nucleotide, in that case, a 2′-O-methylated guanosine.

Accordingly, whenever reference is made to therapeutic coding RNA in the context of the invention, the first nucleotide of said coding RNA or mRNA sequence, that is, the nucleotide downstream of the m7G(5′)ppp structure, may be a 2′-O-methylated guanosine or a 2-O-methylated adenosine.

Stability or efficiency of the RNA can also be effected, e.g., by a modified phosphate backbone of the therapeutic RNA of the first component. A backbone modification may be a modification in which phosphates of the backbone of the nucleotides of the RNA are chemically modified. Nucleotides that may be preferably used comprise e.g. a phosphorothioate-modified phosphate backbone, preferably at least one of the phosphate oxygens contained in the phosphate backbone being replaced by a sulfur atom. Stabilized RNAs may further include, e.g.: non-ionic phosphate analogues, such as, e.g., alkyl and aryl phosphonates, in which the charged phosphonate oxygen is replaced by an alkyl or aryl group, or phosphodiesters and alkylphosphotriesters, in which the charged oxygen residue is present in alkylated form. Such backbone modifications typically include modifications from the group consisting of methylphosphonates, phosphoramidates and phosphorothioates (e.g. cytidine-5′-O-(1-thiophosphate)).

Accordingly, in preferred embodiments, the at least one therapeutic RNA of the first component comprises at least one modified nucleotide and/or at least one nucleotide analogue.

In embodiments, the at least one therapeutic RNA of the first component comprises at least one modified nucleotide, wherein the at least one modified nucleotide is selected from a backbone modified nucleotide, a sugar modified nucleotide and/or a base modified nucleotide or any combinations thereof.

A backbone modification in the context of the invention is a modification in which phosphates of the backbone of the nucleotides are chemically modified. A sugar modification in the context of the invention is a chemical modification of the sugar of the nucleotides of the RNA. A base modification in the context of the invention is a chemical modification of the base moiety of the nucleotides of the RNA. In this context, nucleotide analogues or modifications are preferably selected from nucleotide analogues/modified nucleotides which are applicable for transcription and/or translation. Preferably, nucleotide analogues/modified nucleotides are selected that show reduced stimulation of the innate immune system (after in vivo administration of the RNA comprising such a modified nucleotide).

In embodiments, the nucleotide analogues/modifications which may be incorporated into an RNA as described herein are preferably selected from 2-amino-6-chloropurineriboside-5′-triphosphate, 2-Aminopurine-riboside-5′-triphosphate; 2-aminoadenosine-5′-triphosphate, 2′-Amino-2′-deoxycytidine-triphosphate, 2-thiocytidine-5′-triphosphate, 2-thiouridine-5′-triphosphate, 2′-Fluorothymidine-5′-triphosphate, 2′-O-Methyl-inosine-5′-triphosphate 4-thiouridine-5′-triphosphate, 5-aminoallylcytidine-5′-triphosphate, 5-aminoallyluridine-5′-triphosphate, 5-bromocytidine-5′-triphosphate, 5-bromouridine-5′-triphosphate, 5-Bromo-2′-deoxycytidine-5′-triphosphate, 5-Bromo-2′-deoxyuridine-5′-triphosphate, 5-iodocytidine-5′-triphosphate, 5-Iodo-2′-deoxycytidine-5′-triphosphate, 5-iodouridine-5′-triphosphate, 5-Iodo-2′-deoxyuridine-5′-triphosphate, 5-methylcytidine-5′-triphosphate, 5-methyluridine-5′-triphosphate, 5-Propynyl-2′-deoxycytidine-5′-triphosphate, 5-Propynyl-2′-deoxyuridine-5′-triphosphate, 6-azacytidine-5′-triphosphate, 6-azauridine-5′-triphosphate, 6-chloropurineriboside-5′-triphosphate, 7-deazaadenosine-5′-triphosphate, 7-deazaguanosine-5′-triphosphate, 8-azaadenosine-5′-triphosphate, 8-azidoadenosine-5′-triphosphate, benzimidazole-riboside-5′-triphosphate, N1-methyladenosine-5′-triphosphate, N1-methylguanosine-5′-triphosphate, N6-methyladenosine-5′-triphosphate, 06-methylguanosine-5′-triphosphate, pseudouridine-5′-triphosphate, or puromycin-5′-triphosphate, xanthosine-5′-triphosphate. Particular preference is given to nucleotides for base modifications selected from the group of base-modified nucleotides consisting of 5-methylcytidine-5′-triphosphate, 7-deazaguanosine-5′-triphosphate, 5-bromocytidine-5′-triphosphate, and pseudouridine-5′-triphosphate, pyridin-4-one ribonucleoside, 5-aza-uridine, 2-thio-5-aza-uridine, 2-thiouridine, 4-thio-pseudouridine, 2-thio-pseudouridine, 5-hydroxyuridine, 3-methyluridine, 5-carboxymethyl-uridine, 1-carboxymethyl-pseudouridine, 5-propynyl-uridine, 1-propynyl-pseudouridine, 5-taurinomethyluridine, 1-taurinomethyl-pseudouridine, 5-taurinomethyl-2-thio-uridine, 1-taurinomethyl-4-thio-uridine, 5-methyl-uridine, 1-methyl-pseudouridine, 4-thio-1-methyl-pseudouridine, 2-thio-1-methyl-pseudouridine, 1-methyl-1-deaza-pseudouridine, 2-thio-1-methyl-1-deaza-pseudouridine, dihydrouridine, dihydropseudouridine, 2-thio-dihydrouridine, 2-thio-dihydropseudouridine, 2-methoxyuridine, 2-methoxy-4-thio-uridine, 4-methoxy-pseudouridine, and 4-methoxy-2-thio-pseudouridine, 5-aza-cytidine, pseudoisocytidine, 3-methyl-cytidine, N4-acetylcytidine, 5-formylcytidine, N4-methylcytidine, 5-hydroxymethylcytidine, 1-methyl-pseudoisocytidine, pyrrolo-cytidine, pyrrolo-pseudoisocytidine, 2-thio-cytidine, 2-thio-5-methyl-cytidine, 4-thio-pseudoisocytidine, 4-thio-1-methyl-pseudoisocytidine, 4-thio-1-methyl- 1-deaza-pseudoisocytidine, 1-methyl-1-deaza-pseudoisocytidine, zebularine, 5-aza-zebularine, 5-methyl-zebularine, 5-aza-2-thio-zebularine, 2-thio-zebularine, 2-methoxy-cytidine, 2-methoxy-5-methyl-cytidine, 4-methoxy-pseudoisocytidine, and 4-methoxy-1-methyl-pseudoisocytidine, 2-aminopurine, 2, 6-diaminopurine, 7-deaza-adenine, 7-deaza-8-aza-adenine, 7-deaza-2-aminopurine, 7-deaza-8-aza-2-aminopurine, 7-deaza-2,6-diaminopurine, 7-deaza-8-aza-2,6-diaminopurine, 1-methyladenosine, N6-methyladenosine, N6-isopentenyladenosine, N6-(cis-hydroxyisopentenyl)adenosine, 2-methylthio-N6-(cis-hydroxyisopentenyl) adenosine, N6-glycinylcarbamoyladenosine, N6-threonylcarbamoyladenosine, 2-methylthio-N6-threonyl carbamoyladenosine, N6,N6-dimethyladenosine, 7-methyladenine, 2-methylthio-adenine, and 2-methoxy-adenine, inosine, 1-methyl-inosine, wyosine, wybutosine, 7-deaza-guanosine, 7-deaza-8-aza-guanosine, 6-thio-guanosine, 6-thio-7-deaza-guanosine, 6-thio-7-deaza-8-aza-guanosine, 7-methyl-guanosine, 6-thio-7-methyl-guanosine, 7-methylinosine, 6-methoxy-guanosine, 1-methylguanosine, N2-methylguanosine, N2,N2-dimethylguanosine, 8-oxo-guanosine, 7-methyl-8-oxo-guanosine, 1-methyl-6-thio-guanosine, N2-methyl-6-thio-guanosine, and N2,N2-dimethyl-6-thio-guanosine, 5-O-(1-thiophosphate)-adenosine, 5′-O-(1-thiophosphate)-cytidine, 5′-O-(1-thiophosphate)-guanosine, 5′-O-(1-thiophosphate)-uridine, 5′-O-(1-thiophosphate)-pseudouridine, 6-aza-cytidine, 2-thio-cytidine, alpha-thio-cytidine, Pseudo-iso-cytidine, 5-aminoallyl-uridine, 5-iodo-uridine, N1-methyl-pseudouridine, 5,6-dihydrouridine, alpha-thio-uridine, 4-thio-uridine, 6-aza-uridine, 5-hydroxy-uridine, deoxy-thymidine, 5-methyl-uridine, Pyrrolo-cytidine, inosine, alpha-thio-guanosine, 6-methyl-guanosine, 5-methyl-cytdine, 8-oxo-guanosine, 7-deaza-guanosine, N1-methyl-adenosine, 2-amino-6-Chloro-purine, N6-methyl-2-amino-purine, Pseudo-iso-cytidine, 6-Chloro-purine, N6-methyl-adenosine, alpha-thio-adenosine, 8-azido-adenosine, 7-deaza-adenosine.

In embodiments, the at least one chemical modification is selected from pseudouridine, N1-methylpseudouridine, N1-ethylpseudouridine, 2-thiouridine, 4′-thiouridine, 5-methylcytosine, 5-methyluridine, 2-thio-1-methyl-1-deaza-pseudouridine, 2-thio-1-methyl-pseudouridine, 2-thio-5-aza-uridine, 2-thio-dihydropseudouridine, 2-thio- dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, 5-methoxyuridine and 2′-O-methyl uridine.

In embodiments, 100% of the uracil in the cds of the therapeutic RNA of the first component have a chemical modification, preferably a chemical modification that is in the 5-position of the uracil. In other embodiments, at least 10%, 20%, 30%, 40%, 50%, 60%, 70%, 80%, or 90% of the uracil nucleotides in the cds have a chemical modification, preferably a chemical modification that is in the 5-position of said uracil nucleotides. Such modifications are suitable in the context of the invention, as a reduction of natural uracil may reduce the stimulation of the innate immune system (after in vivo administration of the RNA comprising such a modified nucleotide) potentially caused by the first component upon administration to a cell.

Suitably, the therapeutic RNA of the first component, in particular, the cds of said therapeutic RNA, may comprise at least one modified nucleotide, wherein said at least one modified nucleotide may be selected from pseudouridine (ψ), N1- methylpseudouridine (m1y), 5-methylcytosine, and 5-methoxyuridine, wherein pseudouridine (ψ) is preferred.

In the context of the invention it is preferred that the therapeutic RNA of the first component, preferably the mRNA, comprises a 5′-cap structure as defined herein, preferably a Cap1 structure, and is devoid of any modified nucleotides as defined herein. Accordingly, the therapeutic RNA of the first component may comprise a 5′-cap structure, and an RNA sequence comprising A, U, G, C nucleotides, wherein the RNA sequence is devoid of any modified nucleotides.

In alternative embodiments, the therapeutic RNA of the first component, preferably the mRNA, comprises a 5′-cap structure as defined herein, preferably a Cap1 structure, and additionally comprises modified nucleotides as defined herein, preferably selected from pseudouridine (Lp), N1- methylpseudouridine (m1y), 5-methylcytosine, and 5-methoxyuridine.

In embodiments, the A/U content in the sequence environment of the ribosome binding site of the therapeutic (coding) RNA may be increased compared to the A/U content in the environment of the ribosome binding site of its respective wild type nucleic acid. This modification (an increased A/U content around the ribosome binding site) increases the efficiency of ribosome binding to the RNA. An effective binding of the ribosomes to the ribosome binding site in turn has the effect of an efficient translation of the RNA.

Accordingly, in particularly preferred embodiments, the therapeutic (coding) RNA of the first component comprises a ribosome binding site, also referred to as “Kozak sequence” identical to or at least 80%, 85%, 90%, 95% identical to any one of the sequences SEQ ID NOs: 3 or 4, or fragments or variants thereof.

In preferred embodiments, the at least one therapeutic RNA of the first component, preferably the mRNA, comprises at least one poly(A) sequence, and/or at least one poly(C) sequence, and/or at least one histone stem-loop sequence/structure.

Accordingly, the therapeutic (coding) RNA of the first component may comprise at least one poly(N) sequence, e.g. at least one poly(A) sequence, at least one poly(U) sequence, at least one poly(C) sequence, or combinations thereof.

In preferred embodiments, the therapeutic (coding) RNA comprises at least one poly(A) sequence.

The terms “poly(A) sequence”, “poly(A) tail” or “3-poly(A) tail” as used herein will be recognized and understood by the person of ordinary skill in the art, and are e.g. intended to be a sequence of adenosine nucleotides, typically located at the 3′-end of a coding RNA, of up to about 1000 adenosine nucleotides. Said poly(A) sequence is essentially homopolymeric, e.g. a poly(A) sequence of e.g. 100 adenosine nucleotides has essentially the length of 100 nucleotides. In other embodiments, the poly(A) sequence may be interrupted by at least one nucleotide different from an adenosine nucleotide.

The poly(A) sequence, suitable located downstream of a 3′ UTR as defined herein, may comprise about 10 to about 500 adenosine nucleotides, about 30 to about 500 adenosine nucleotides, about 30 to about 200 adenosine nucleotides, or about 50 to about 150 adenosine nucleotides. Suitably, the length of the poly(A) sequence may be at least about or even more than about 30, 50, 64, 75, 100, 200, 300, 400, or 500 adenosine nucleotides. In preferred embodiments, the poly(A) sequence comprises about 50 to about 250 adenosines. In particularly preferred embodiments, the poly(A) sequence comprises about 64 adenosine nucleotides. In particularly preferred embodiments, the poly(A) sequence comprises about 100 adenosine nucleotides.

The poly(A) sequence as defined herein is suitably located at the 3′ terminus of the therapeutic RNA (e.g. the mRNA). Accordingly it is preferred that the 3′ terminal nucleotide of the RNA (that is the last 3′ terminal nucleotide in the polynucleotide chain) is the 3′ terminal A nucleotide of the at least one poly(A) sequence. The term “located at the 3′ terminus” has to be understood as being located exactly at the 3′ terminus—in other words, the 3′ terminus of the RNA consists of a poly(A) sequence terminating with an A nucleotide.

Preferably, the poly(A) sequence of the therapeutic RNA of the first component is obtained from a DNA template during RNA in vitro transcription. In other embodiments, the poly(A) sequence is obtained in vitro by common methods of chemical synthesis without being necessarily transcribed from a DNA template. In other embodiments, poly(A) sequences are generated by enzymatic polyadenylation of the RNA (after RNA in vitro transcription) using commercially available polyadenylation kits and corresponding protocols known in the art, or alternatively, by using immobilized poly(A)polymerases e.g. using a methods and means as described in WO2016/174271.

Accordingly, the therapeutic RNA may comprise a poly(A)sequence obtained by enzymatic polyadenylation, wherein the majority of RNA molecules comprise about 100 (+/−10) to about 500 (+/−50), preferably about 250 (+/−25) adenosine nucleotides.

In embodiments, the therapeutic RNA may comprise a poly(A) sequence derived from a template DNA and may comprise at least one additional poly(A) sequence generated by enzymatic polyadenylation, as described in WO2016/091391.

In embodiments, the therapeutic RNA of the first component may comprise at least one poly(C) sequence.

In embodiments, the poly(C) sequence, suitably located at the 3′ terminus or in proximity to 3′ terminus, comprises about 10 to 200 cytosine nucleotides, about 10 to 100 cytosine nucleotides, or about 10 to 50 cytosine nucleotides. In preferred embodiments, the poly(C) sequence comprises about 30 cytosine nucleotides.

In preferred embodiments, the therapeutic RNA of the first component comprises at least one histone stem-loop.

The term “histone stem-loop” (abbreviated as “hsl”) as used herein will be recognized and understood by the person of ordinary skill in the art, and is e.g. intended to refer to nucleic acid sequences predominantly found in histone mRNAs.

Histone stem-loop sequences/structures may suitably be selected from histone stem-loop sequences as disclosed in WO2012/019780, the disclosure relating to histone stem-loop sequences/histone stem-loop structures incorporated herewith by reference. A histone stem-loop sequence that may be used within the present invention may preferably be derived from formulae (I) or (II) of WO2012/019780. According to a further preferred embodiment the coding RNA may comprise at least one histone stem-loop sequence derived from at least one of the specific formulae (Ia) or (IIa) of the patent application WO2012/019780.

In particularly preferred embodiment, the therapeutic RNA of the first component comprises at least one histone stem-loop sequence, wherein said histone stem-loop sequence comprises a nucleic acid sequence identical or at least 70%, 80%, 90%, 91%, 92%, 93%, 94%, 95%, 96%, 97%, 98%, or 99% identical to SEQ ID NOs: 1 or 2, or fragments or variants thereof.

In embodiments, the therapeutic RNA of the first component comprises a 3′-terminal sequence element. Said 3′-terminal sequence element comprises a poly(A)sequence and a histone-stem-loop sequence, and optionally a poly(C) sequence, wherein said sequence element is located at the 3′ terminus of the RNA of the invention.

Accordingly, the therapeutic RNA of the first component may comprise a 3′-terminal sequence element comprising or consisting of a nucleic acid sequence being identical or at least 70%, 80%, 90%, 91%, 92%, 93%, 94%, 95%, 96%, 97%, 98%, or 99% identical to SEQ ID NOs: 7 to 38, or a fragment or variant thereof.

In various embodiments, therapeutic RNA of the first component may comprise a 5′-terminal sequence element according to SEQ ID NOs: 5 or 6, or a fragment or variant thereof. Such a 5′-terminal sequence element comprises e.g. a binding site for T7 RNA polymerase. Further, the first nucleotide of said 5′-terminal start sequence may preferably comprise a 2′-O-methylation, e.g. 2-O-methylated guanosine or a 2-O-methylated adenosine.

The therapeutic RNA of the first component, preferably the mRNA, may comprise a cds, a 5′-UTR and/or a 3′-UTR. UTRs (untranslated region) may harbor regulatory sequence elements or motifs that determine RNA turnover, stability, and/or localization. UTRs may also harbor sequence elements or motifs that enhance translation. In medical application of RNA, translation of the cds into at least one peptide or protein is of paramount importance to therapeutic efficacy. Certain combinations of 3′-UTRs and/or 5′-UTRs can enhance expression of operably linked coding sequences encoding peptides or proteins as defined above. RNA harboring said UTR combinations advantageously enable rapid and transient expression of encoded peptides or proteins after administration to a subject.

Accordingly, therapeutic RNA of the first component, preferably the mRNA may comprise certain combinations of 3′-UTRs and/or 5′-UTRs, resulting in (improved) translation of a therapeutic protein (e.g., CRISPR-associated endonuclease, or antigen), and hence, in expression of the protein in therapeutically relevant cells or tissues.

In preferred embodiments, the therapeutic RNA of the first component, preferably the mRNA, comprises at least one heterologous 5′-UTR and/or at least one heterologous 3′-UTR. Said 5′-UTRs or 3′-UTRs may be derived from naturally occurring genes or may be synthetically engineered. In preferred embodiments, the RNA comprises at least one cds operably linked to at least one (heterologous) 3′-UTR and/or at least one (heterologous) 5-UTR.

In preferred embodiments, the therapeutic RNA of the first component comprises at least one heterologous 3′-UTR.

The term “3′-untranslated region” or “3′-UTR” or “3′-UTR element” will be recognized and understood by the person of ordinary skill in the art, and are e.g. intended to refer to a part of the RNA, located 3′ (i.e. downstream) of a cds, which is not translated into protein. A 3′-UTR may be part of an RNA, e.g. an mRNA, located between a cds and a terminal poly(A) sequence. A 3′-UTR may comprise elements for controlling gene expression, also called regulatory elements. Such regulatory elements may be, e.g., ribosomal binding sites, miRNA binding sites etc.

Preferably, the therapeutic RNA of the first component, preferably the mRNA, comprises a 3′-UTR, which may be derivable from a gene that relates to RNA with enhanced half-life (i.e. that provides a stable RNA).

In some embodiments, a 3′-UTR comprises one or more of a polyadenylation signal, a binding site for proteins that affect an RNA stability of location in a cell, or one or more miRNA or binding sites for miRNAs.

MicroRNAs (or miRNA) are 19-25 nucleotide long noncoding RNAs that bind to the 3′-UTR of nucleic acid molecules and down-regulate gene expression either by reducing nucleic acid molecule stability or by inhibiting translation. E.g., microRNAs are known to regulate RNA, and thereby protein expression, e.g. in liver (miR-122), heart (miR-Id, miR-149), endothelial cells (miR-17-92, miR-126), adipose tissue (let-7, miR-30c), kidney (miR-192, miR-194, miR-204), myeloid cells (miR-142-3p, miR-142-5p, miR-16, miR-21, miR-223, miR-24, miR-27), muscle (miR-133, miR-206, miR-208), and lung epithelial cells (let-7, miR-133, miR-126). The therapeutic RNA of the first component may comprise one or more microRNA target sequences, microRNA sequences, or microRNA seeds. Such sequences may e.g. correspond to any known microRNA such as those taught in US2005/0261218 and US2005/0059005.

Accordingly, miRNA, or binding sites for miRNAs as defined above may be removed from the 3′-UTR or introduced into the 3′-UTR in order to tailor the expression or the activity of the therapeutic RNA to desired cell types or tissues.

In preferred embodiments, the therapeutic RNA of the first component, preferably the mRNA, comprises at least one heterologous 3′-UTR, wherein the at least one heterologous 3′-UTR comprises a nucleic acid sequence derived from a 3′-UTR of a gene selected from PSMB3, ALB7, alpha-globin (referred to as “muag”), CASP1, COX6B1, GNAS, NDUFA1 and RPS9, or from a homolog, a fragment or variant of any one of these genes.

Particularly preferred nucleic acid sequences in that context can be derived from published PCT application WO2019/077001A1, in particular, claim 9 of WO2019/077001A1. The corresponding 3′-UTR sequences of claim 9 of WO2019/077001A1 are herewith incorporated by reference (e.g., SEQ ID NOs: 23 to 34 of WO2019/077001A1, or fragments or variants thereof).

In other embodiments, the therapeutic RNA of the first component, preferably the mRNA, comprises a 3′-UTR as described in WO2016/107877, the disclosure of WO2016/107877 relating to 3′-UTR sequences herewith incorporated by reference. Suitable 3′-UTRs are SEQ ID NOs: 1 to 24 and SEQ ID NOs: 49 to 318 of WO2016/107877, or fragments or variants of these sequences. In other embodiments, the therapeutic RNA comprises a 3′-UTR as described in WO2017/036580, the disclosure of WO2017/036580 relating to 3′-UTR sequences herewith incorporated by reference. Suitable 3′-UTRs are SEQ ID NOs: 152 to 204 of WO2017/036580, or fragments or variants of these sequences. In other embodiments, the therapeutic RNA comprises a 3′-UTR as described in WO2016/022914, the disclosure of WO2016022914 relating to 3′-UTR sequences herewith incorporated by reference. Particularly preferred 3′-UTRs are nucleic acid sequences according to SEQ ID NOs: 20 to 36 of WO2016/022914, or fragments or variants of these sequences.

In preferred embodiments, the coding RNA of the composition for use comprises at least one heterologous 5′-UTR.

The terms “5′-untranslated region” or “5′-UTR” or “5′-UTR element” will be recognized and understood by the person of ordinary skill in the art, and are e.g. intended to refer to a part of the RNA, located 5′ (i.e. “upstream”) of a cds, which is not translated into protein. A 5′-UTR may be part of an RNA located 5′ of the cds. Typically, a 5′-UTR starts with the transcriptional start site and ends before the start codon of the cds. A 5′-UTR may comprise elements for controlling gene expression, called regulatory elements. Such regulatory elements may be, e.g., ribosomal binding sites, miRNA binding sites etc. The 5′-UTR may be post-transcriptionally modified, e.g. by enzymatic or post-transcriptional addition of a 5′-cap structure (see above).

Preferably, the therapeutic RNA of the first component, preferably the mRNA, comprises a 5′-UTR, which may be derivable from a gene that relates to an RNA with enhanced half-life (i.e. that provides a stable RNA).

In some embodiments, a 5′-UTR comprises one or more of a binding site for proteins that affect an RNA stability of location in a cell, or one or more miRNA or binding sites for miRNAs (as defined above).

Accordingly, miRNA or binding sites for miRNAs as defined above may be removed from the 5′-UTR or introduced into the 5′-UTR in order to tailor the expression or activity of the therapeutic RNA to desired cell types or tissues.

In preferred embodiments, the therapeutic RNA of the first component, preferably the mRNA, comprises at least one heterologous 5′-UTR, wherein the at least one heterologous 5′-UTR comprises a nucleic acid sequence derived from a human and/or murine 5′-UTR of gene selected from HSD17B4, RPL32, ASAH1, ATP5A1, MP68, NDUFA4, NOSIP, RPL31, SLC7A3, TUBB4B, and UBQLN2, or from a homolog, a fragment or variant of any one of these genes. Particularly preferred nucleic acid sequences in that context can be derived from published PCT application WO2019/077001A1, in particular, claim 9 of WO2019/077001A1. The corresponding 5′-UTR sequences of claim 9 of WO2019/077001A1 are herewith incorporated by reference (e.g., SEQ ID NOs: 1-20 of WO2019/077001A1, or fragments or variants thereof).

Suitably, in preferred embodiments, the therapeutic RNA of the first component, preferably the mRNA, comprises at least one cds encoding at least one peptide or protein as specified herein, operably linked to a 3′-UTR and/or a 5′-UTR selected from the following 5′-UTR/3′-UTR combinations: a-1 (HSD17B4/PSMB3), a-2 (NDUFA4/PSMB3), a-3 (SLC7A3/PSMB3), a-4 (NOSIP/PSMB3), a-5 (MP68/PSMB3), b-1 (UBQLN2/RPS9), b-2 (ASAH1/RPS9), b-3 (HSD17B4/RPS9), b-4 (HSD17B4/CASP1), b-5 (NOSIP/COX6B1), c-1 (NDUFA4/RPS9), c-2 (NOSIP/NDUFA1), c-3 (NDUFA4/COX6B1), c-4 (NDUFA4/NDUFA1), c-5 (ATP5A1/PSMB3), d-1 (Rpl31/PSMB3), d-2 (ATP5A1/CASP1), d-3 (SLC7A3/GNAS), d-4 (HSD17B4/NDUFA1), d-5 (Slc7a3/Ndufa1), e-1 (TUBB4B/RPS9), e-2 (RPL31/RPS9), e-3 (MP68/RPS9), e-4 (NOSIP/RPS9), e-5 (ATP5A1/RPS9), e-6 (ATP5A1/COX6B1), f-1 (ATP5A1/GNAS), f-2 (ATP5A1/NDUFA1), f-3 (HSD17B4/COX6B1), f-4 (HSD17B4/GNAS), f-5 (MP68/COX6B1), g-1 (MP68/NDUFA1), g-2 (NDUFA4/CASP1), g-3 (NDUFA4/GNAS), g-4 (NOSIP/CASP1), g-5 (RPL31/CASP1), h-1 (RPL31/COX6B1), h-2 (RPL31/GNAS), h-3 (RPL31/NDUFA1), h-4 (Slc7a3/CASP1), h-5 (SLC7A3/COX6B1), i-1 (SLC7A3/RPS9), i-2 (RPL32/ALB7), i-2 (RPL32/ALB7), or i-3 (a-globin gene/-).

In that context, suitable 5′-UTR sequences as defined above may be or may be derived from SEQ ID NOs: 44-65, or fragment or variants thereof, and suitable 3′-UTR sequences as defined above may be or may be derived from SEQ ID NOs: 66-81, 185, 186.

In other embodiments, the therapeutic RNA of the first component, preferably the mRNA, comprises a 5′-UTR as described in WO2013/143700, the disclosure of WO2013/143700 relating to 5′-UTR sequences herewith incorporated by reference. Particularly preferred 5′-UTRs are nucleic acid sequences derived from SEQ ID NOs: 1-1363, SEQ ID NO: 1395, SEQ ID NO: 1421 and SEQ ID NO: 1422 of WO2013/143700, or fragments or variants of these sequences. In other embodiments, the therapeutic RNA comprises a 5′-UTR as described in WO2016/107877, the disclosure of WO2016/107877 relating to 5′-UTR sequences herewith incorporated by reference. Particularly preferred 5′-UTRs are nucleic acid sequences according to SEQ ID NOs: 25 to 30 and SEQ ID NOs: 319 to 382 of WO2016/107877, or fragments or variants of these sequences. In other embodiments, the therapeutic RNA comprises a 5′-UTR as described in WO2017/036580, the disclosure of WO2017/036580 relating to 5′-UTR sequences herewith incorporated by reference. Particularly preferred 5′-UTRs are nucleic acid sequences according to SEQ ID NOs: 1 to 151 of WO2017/036580, or fragments or variants of these sequences. In other embodiments, the therapeutic RNA comprises a 5′-UTR as described in WO2016/022914, the disclosure of WO2016/022914 relating to 5′-UTR sequences herewith incorporated by reference. Particularly preferred 5′-UTRs are nucleic acid sequences according to SEQ ID NOs: 3 to 19 of WO2016/022914, or fragments or variants of these sequences.

In embodiments therapeutic RNA of the first component, preferably the mRNA, comprises the following elements preferably in 5′- to 3-direction:

A) 5′-cap structure, preferably m7G(5′)ppp(5′)(2′OMeA) or m7G(5′)ppp(5′)(2′OMeG);

B) 5-terminal start element, preferably selected from SEQ ID NOs: 5 or 6 or fragments or variants thereof;

C) optionally, 5′-UTR, preferably as specified herein, for example selected from SEQ ID NOs: 44 to 65;

D) a ribosome binding site, preferably selected from SEQ ID NOs: 3 or 4 or fragments or variants thereof;

E) at least one coding sequence encoding at least one therapeutic peptide or protein as specified herein;

F) 3′-UTR preferably as specified herein, for example selected from SEQ ID NOs: 66 to 81;

G) optionally, poly(A) sequence comprising about 50 to about 500 adenosines;

H) optionally, poly(C) sequence comprising about 10 to about 100 cytosines;

I) optionally, histone stem-loop (sequence), preferably selected from SEQ ID NOs: 1 or 2;

J) optionally, 3′-terminal sequence element SEQ ID NOs: 7 to 38.

Preferably, the therapeutic RNA of the first component, preferably the mRNA, comprises about 50 to about 20000 nucleotides, or about 500 to about 10000 nucleotides, or about 1000 to about 10000 nucleotides, or preferably about 1000 to about 5000 nucleotides.

In one embodiment, the first component (e.g. the therapeutic RNA) and the second component (e.g. a nucleic acid antagonist) are attached to each other.

Advantageously, such an attachment may simplify the co-formulation in a carrier (see described below). Ideally, the first and the second component are attached to each other via non-covalent binding to allow detachment after administration in vivo. Accordingly, the invention also relates to a compound comprising the first component as defined herein and the second component as defined herein.

Formulation of the First and/or the Second Component:

In the following, advantageous embodiments and features regarding the formulation/complexation of the at least one antagonist of at least one RNA sensing pattern recognition receptor of the second component are described. Further, advantageous embodiments and features regarding formulation/complexation of the at least one therapeutic RNA of the first component are described. All described embodiments and features regarding formulation in the context of a “combination” (first aspect) are likewise be applicable to the “composition” (second aspect) or the “kit or kit of parts” (third aspect).

In a preferred embodiment, the nucleic acid of the second component as defined herein and/or the at least one therapeutic RNA of the first component as defined herein, is complexed or associated with or at least partially complexed or partially associated with one or more cationic or polycationic compound, preferably cationic or polycationic polymer, cationic or polycationic polysaccharide, cationic or polycationic lipid, cationic or polycationic protein, or cationic or polycationic peptide, or any combinations thereof.

In an embodiments, the nucleic acid of the second component as defined herein is attached to one or more cationic or polycationic compounds, preferably cationic or polycationic polymers, cationic or polycationic polysaccharide, cationic or polycationic lipid, cationic or polycationic protein, or cationic or polycationic peptide, or any combinations thereof. Suitably, the therapeutic RNA of the second component is complexed or associated with such a cationic or polycationic compound.

The term “cationic or polycationic compound” as used herein will be recognized and understood by the person of ordinary skill in the art, and are e.g. intended to refer to a charged molecule, which is positively charged at a pH value ranging from about 1 to 9, at a pH value ranging from about 3 to 8, at a pH value ranging from about 4 to 8, at a pH value ranging from about 5 to 8, more preferably at a pH value ranging from about 6 to 8, even more preferably at a pH value ranging from about 7 to 8, most preferably at a physiological pH, e.g. ranging from about 7.2 to about 7.5. Accordingly, a cationic component, e.g. a cationic peptide, cationic protein, cationic polymer, cationic polysaccharide, cationic lipid may be any positively charged compound or polymer which is positively charged under physiological conditions. A “cationic or polycationic peptide or protein” may contain at least one positively charged amino acid, or more than one positively charged amino acid, e.g. selected from Arg, His, Lys or Orn. Accordingly, “polycationic” components are also within the scope exhibiting more than one positive charge under the given conditions.

Cationic or polycationic compounds, being particularly preferred may be selected from the following list of cationic or polycationic peptides or proteins of fragments thereof: protamine, nucleoline, spermine or spermidine, or other cationic peptides or proteins, such as poly-L-lysine (PLL), poly-arginine, basic polypeptides, cell penetrating peptides (CPPs), including HIV-binding peptides, HIV-1 Tat (HIV), Tat-derived peptides, Penetratin, VP22 derived or analogue peptides, HSV VP22 (Herpes simplex), MAP, KALA or protein transduction domains (PTDs), PpT620, prolin-rich peptides, arginine-rich peptides, lysine-rich peptides, MPG-peptide(s), Pep-1, L-oligomers, Calcitonin peptide(s), Antennapedia-derived peptides, pAntp, plsl, FGF, Lactoferrin, Transportan, Buforin-2, Bac715-24, SynB, SynB(1), pVEC, hCT-derived peptides, SAP, or histones. More preferably, the coding RNA is complexed with one or more polycations, preferably with protamine or oligofectamine, most preferably with protamine.

Further preferred cationic or polycationic compounds, which can be used as complexation agent for the first and/or the second component may include cationic polysaccharides, e.g. chitosan, polybrene etc.; cationic lipids, e.g. DOTMA, DMRIE, di-C14-amidine, DOTIM, SAINT, DC-Chol, BGTC, CTAP, DOPC, DODAP, DOPE: Dioleyl phosphatidylethanol-amine, DOSPA, DODAB, DOIC, DMEPC, DOGS, DIMRI, DOTAP, DC-6-14, CLIP1, CLIP6, CLIP9, oligofectamine; or cationic or polycationic polymers, e.g. modified polyaminoacids, such as beta-aminoacid-polymers or reversed polyamides, etc., modified polyethylenes, such as PVP etc., modified acrylates, such as pDMAEMA etc., modified amidoamines such as pAMAM etc., modified polybetaaminoester (PBAE), such as diamine end modified 1,4 butanediol diacrylate-co-5-amino-1-pentanol polymers, etc., dendrimers, such as polypropylamine dendrimers or pAMAM based dendrimers, etc., polyimine(s), such as PEI, poly(propyleneimine), etc., polyallylamine, sugar backbone based polymers, such as cyclodextrin based polymers, dextran based polymers, etc., silan backbone based polymers, such as PMOXA-PDMS copolymers, etc., blockpolymers consisting of a combination of one or more cationic blocks (e.g. selected from a cationic polymer as mentioned above) and of one or more hydrophilic or hydrophobic blocks (e.g. polyethyleneglycole); etc.

Preferred cationic or polycationic proteins or peptides that may be used for complexation of the first and/or the second component can be derived from formula (Arg)l;(Lys)m;(His)n;(Orn)o;(Xaa)x of the patent application WO2009/030481 or WO2011/026641, the disclosure of WO2009/030481 or WO2011/026641 relating thereto incorporated herewith by reference.

In various embodiments, the one or more cationic or polycationic peptides of the first and/or second component are selected from SEQ ID NO: 39 to 43, or any combinations thereof.

Accordingly, in preferred embodiments, the at least one antagonist of the second component, preferably the nucleic acid, is complexed or associated with or at least partially complexed or partially associated with one or more cationic or polycationic peptides selected from SEQ ID NO: 39 to 43, or any combinations thereof.

Accordingly, in preferred embodiments, the at least one therapeutic RNA of the first component, preferably the mRNA, is complexed or associated with or at least partially complexed or partially associated with one or more cationic or polycationic peptides selected from SEQ ID NO: 39 to 43, or any combinations thereof.

In embodiments, the nucleic acid of the second component as defined herein is complexed or associated with or at least partially complexed or partially associated with one or more cationic or polycationic polymer.

In embodiments, the at least one therapeutic RNA of the first component, preferably the mRNA, is complexed or associated with or at least partially complexed or partially associated with one or more cationic or polycationic polymer.

Accordingly, in embodiments, the first and/or second component comprises at least one polymeric carrier.

The term “polymeric carrier” as used herein will be recognized and understood by the person of ordinary skill in the art, and is e.g. intended to refer to a compound that facilitates transport and/or complexation of another compound (e.g. first, second component). A polymeric carrier is typically a carrier that is formed of a polymer. A polymeric carrier may be associated to its cargo (e.g. RNA) by covalent or non-covalent interaction. A polymer may be based on different subunits, such as a copolymer.

Suitable polymeric carriers in that context may include, e.g., polyacrylates, polyalkycyanoacrylates, polylactide, polylactide-polyglycolide copolymers, polycaprolactones, dextran, albumin, gelatin, alginate, collagen, chitosan, cyclodextrins, protamine, PEGylated protamine, PEGylated PLL and polyethylenimine (PEI), dithiobis(succinimidylpropionate) (DSP), Dimethyl-3,3′-dithiobispropionimidate (DTBP), poly(ethylene imine) biscarbamate (PEIC), poly(L-lysine) (PLL), histidine modified PLL, poly(N-vinylpyrrolidone) (PVP), poly(propylenimine (PPI), poly(amidoamine) (PAMAM), poly(amido ethylenimine) (SS-PAEI), triehtylenetetramine (TETA), poly(β-aminoester), poly(4-hydroxy-L-proine ester) (PHP), poly(allylamine), poly(a-[4-aminobutyl]-L-glycolic acid (PAGA), Poly(D,L-lactic-co-glycolid acid (PLGA), Poly(N-ethyl-4-vinylpyridinium bromide), poly(phosphazene)s (PPZ), poly(phosphoester)s (PPE), poly(phosphoramidate)s (PPA), poly(N-2-hydroxypropylmethacrylamide) (pHPMA), poly(2-(dimethylamino)ethyl methacrylate) (pDMAEMA), poly(2-aminoethyl propylene phosphate) PPE_EA), galactosylated chitosan, N-dodecylated chitosan, histone, collagen and dextran-spermine. In one embodiment, the polymer may be an inert polymer such as, but not limited to, PEG. In one embodiment, the polymer may be a cationic polymer such as, but not limited to, PEI, PLL, TETA, poly(allylamine), Poly(N-ethyl-4-vinylpyridinium bromide), pHPMA and pDMAEMA. In one embodiment, the polymer may be a biodegradable PEI such as, but not limited to, DSP, DTBP and PEIC. In one embodiment, the polymer may be biodegradable such as, but not limited to, histine modified PLL, SS-PAEI, poly((3-aminoester), PHP, PAGA, PLGA, PPZ, PPE, PPA and PPE-EA.

A suitable polymeric carrier may be a polymeric carrier formed by disulfide-crosslinked cationic compounds. The disulfide-crosslinked cationic compounds may be the same or different from each other. The polymeric carrier can also contain further components (e.g. lipidoid compound). The polymeric carrier used according to the present invention may comprise mixtures of cationic peptides, proteins or polymers and optionally further components as defined herein, which are crosslinked by disulfide bonds (via —SH groups).

In this context, polymeric carriers according to formula (Ia) {(Arg)l;(Lys)m;(His)n;(Orn)o;(Xaa′)x(Cys)y} and formula (Ib) Cys{(Arg)l;(Lys)m;(His)n;(Orn)o;(Xaa)x}Cys of published PCT application WO2012/013326 are preferred, the disclosure of WO2012/013326 relating thereto incorporated herewith by reference.

In embodiments, the polymeric carrier used to complex the at least one coding RNA may be derived from a polymeric carrier molecule according formula (L-P¹-S-[S-P²-S]ₙ-S—P³-L) of published PCT application WO2011/026641, the disclosure of WO2011/026641 relating thereto incorporated herewith by reference.

In embodiments, the polymeric carrier compound is formed by, or comprises, or consists of the peptide elements CysArg12Cys (SEQ ID NO: 39) or CysArg12 (SEQ ID NO: 40) or TrpArg12Cys (SEQ ID NO: 41). In other embodiments, the polymeric carrier compound is formed by, or comprises, or consists of the peptide elements according to SEQ ID NO: 42 or 43.

In particularly preferred embodiments, the polymeric carrier compound consists of a (R₁₂C)-(R₁₂C) dimer, a (WR₁₂C)-(WR₁₂C) dimer, or a (CR₁₂)-(CR₁₂C)-(CR₁₂) trimer, wherein the individual peptide elements in the dimer (e.g. (WR₁₂C)), or the trimer (e.g. (CR₁₂)), are connected via —SH groups.

In preferred embodiments, the cationic or polycationic polymer of the first and/or second component is a polyethylene glycol/peptide polymer comprising HO-PEG5000-S-(S—CHHHHHHRRRRHHHHHHC-S-)7-S-PEG5000-OH (SEQ ID NO: 42 of the peptide monomer) and/or a polyethylene glycol/peptide polymer comprising HO-PEG5000-S-(S-CGHHHHHRRRRHHHHHGC-S-)4-S-PEG5000-OH (SEQ ID NO: 43 of the peptide monomer).

In embodiments, the first and/or second component is complexed or associated with polymeric carriers and, optionally, with at least one lipid or lipidoid as described in published PCT applications WO2017/212008A1, WO2017/212006A1, WO2017/212007A1, and WO2017/212009A1, the disclosures of WO2017/212008A1, WO2017/212006A1, WO2017/212007A1, and WO2017/212009A1 herewith incorporated by reference.

In particularly preferred embodiments, the polymeric carrier (of the first and/or second component) is a peptide polymer, preferably a polyethylene glycol/peptide polymer as defined above, and a lipid, preferably a lipidoid.

A lipidoid (or lipidoit) is a lipid-like compound, i.e. an amphiphilic compound with lipid-like physical properties. The lipidoid preferably comprises two or more cationic nitrogen atoms and at least two lipophilic tails. In contrast to many conventional cationic lipids, the lipidoid may be free of a hydrolysable linking group, in particular linking groups comprising hydrolysable ester, amide or carbamate groups. The cationic nitrogen atoms of the lipidoid may be cationisable or permanently cationic, or both types of cationic nitrogens may be present in the compound. In the context of the present invention the term lipid is considered to also encompass lipidoids.

In some embodiments of the inventions, the lipidoid may comprise a PEG moiety.

Suitably, the lipidoid is cationic, which means that it is cationisable or permanently cationic. In one embodiment, the lipidoid is cationisable, i.e. it comprises one or more cationisable nitrogen atoms, but no permanently cationic nitrogen atoms. In another embodiment, at least one of the cationic nitrogen atoms of the lipidoid is permanently cationic. Optionally, the lipidoid comprises two permanently cationic nitrogen atoms, three permanently cationic nitrogen atoms, or even four or more permanently cationic nitrogen atoms.

In embodiments, the lipidoid may be any one selected from the lipidoids of the lipidoids provided in the table of page 50-54 of published PCT patent application WO2017/212009A1, the specific lipidoids provided in said table, and the specific disclosure relating thereto herewith incorporated by reference.

In preferred embodiments, the lipidoid may be any one selected from 3-C12-OH, 3-C12-OH-cat, 3-C12-amide, 3-C12-amide monomethyl, 3-C12-amide dimethyl, RevPEG(10)-3-C12-OH, RevPEG(10)-DLin-pAbenzoic, 3C12amide-TMA cat., 3C12amide-DMA, 3C12amide-NH2, 3C12amide-OH, 3C12Ester-OH, 3C12 Ester-amin, 3C12Ester-DMA, 2C12Amid-DMA, 3C12-lin-amid-DMA, 2C12-sperm-amid-DMA, or 3C12-sperm-amid-DMA (see table of published PCT patent application WO2017/212009A1 (pages 50-54)). Particularly preferred lipidoids in the context of the invention are 3-C12-OH or 3-C12-OH-cat.

In preferred embodiments, the peptide polymer comprising a lipidoid as specified above, is used to complex the at least one therapeutic RNA of the first component and/or the at least one antagonist of the second component (e.g. nucleic acid) to form complexes having an N/P ratio from about 0.1 to about 20, or from about 0.2 to about 15, or from about 2 to about 15, or from about 2 to about 12, wherein the N/P ratio is defined as the mole ratio of the nitrogen atoms of the basic groups of the cationic peptide or polymer to the phosphate groups of the nucleic acid. In that context, the disclosure of published PCT patent application WO2017/212009A1, in particular claims 1 to 10 of WO2017/212009A1, and the specific disclosure relating thereto is herewith incorporated by reference.

In specific embodiments, the at least one therapeutic RNA of the first component, preferably the mRNA, is complexed or associated with a polymeric carrier, preferably with a polyethylene glycol/peptide polymer as defined above, and a lipidoid, preferably 3-C12-OH and/or 3-C12-OH-cat.

In specific embodiments, the at least one antagonist of the second component, preferably the nucleic acid, is complexed or associated with a polymeric carrier, preferably with a polyethylene glycol/peptide polymer as defined above, and a lipidoid, preferably 3-C12-OH and/or 3-C12-OH-cat.

Further suitable lipidoids may be derived from published PCT patent application WO2010/053572. In particular, lipidoids derivable from claims 1 to 297 of published PCT patent application WO2010/053572 may be used in the context of the invention, e.g. incorporated into the peptide polymer as described herein, or e.g. incorporated into the lipid nanoparticle (as described below). Accordingly, claims 1 to 297 of published PCT patent application WO2010/053572, and the specific disclosure relating thereto, is herewith incorporated by reference.

In preferred embodiments, the at least one therapeutic RNA of the first compound, preferably the mRNA is complexed, partially complexed, encapsulated, partially encapsulated, or associated with one or more lipids (e.g. cationic lipids and/or neutral lipids), thereby forming liposomes, lipid nanoparticles (LNPs), lipoplexes, and/or nanoliposomes.

In preferred embodiments, the at least one antagonist of the second compound, preferably the nucleic acid, is complexed, partially complexed, encapsulated, partially encapsulated, or associated with one or more lipids (e.g. cationic lipids and/or neutral lipids), thereby forming liposomes, lipid nanoparticles (LNPs), lipoplexes, and/or nanoliposomes.

The liposomes, lipid nanoparticles (LNPs), lipoplexes, and/or nanoliposomes—incorporated therapeutic RNA of the first compound or antagonist (e.g. nucleic acid) of the second compound—may be completely or partially located in the interior space of the liposomes, lipid nanoparticles (LNPs), lipoplexes, and/or nanoliposomes, within the membrane, or associated with the exterior surface of the membrane. The incorporation of said therapeutic RNA of the first compound, or said antagonist of the second compound is also referred to herein as “encapsulation” wherein the therapeutic RNA as defined/antagonist (e.g. nucleic acid) as defined is entirely contained within the interior space of the liposomes, lipid nanoparticles (LNPs), lipoplexes, and/or nanoliposomes. The purpose of incorporating the first and/or the second component into liposomes, lipid nanoparticles (LNPs), lipoplexes, and/or nanoliposomes is to protect the components from an environment which may contain enzymes or chemicals that degrade e.g. the therapeutic RNA and/or systems or receptors that cause the rapid excretion of therapeutic RNA. Moreover, incorporating the first and/or the second component into liposomes, lipid nanoparticles (LNPs), lipoplexes, and/or nanoliposomes may promote the uptake of the RNA, and hence, may enhance their therapeutic effects.

In this context, the terms “complexed” or “associated” refer to the essentially stable combination of the therapeutic RNA of the first component as defined herein, or the antagonist of the second component (e.g. nucleic acid) as defined herein, with one or more lipids into larger complexes or assemblies without covalent binding.

The term “lipid nanoparticle”, also referred to as “LNP”, is not restricted to any particular morphology, and includes any morphology generated when a cationic lipid and optionally one or more further lipids are combined, e.g. in an aqueous environment and/or in the presence of RNA. E.g., a liposome, a lipid complex, a lipoplex and the like are within the scope of an LNP.

Liposomes, lipid nanoparticles (LNPs), lipoplexes, and/or nanoliposomes can be of different sizes such as, but not limited to, a multilamellar vesicle (MLV) which may be hundreds of nanometers in diameter and may contain a series of concentric bilayers separated by narrow aqueous compartments, a small unicellular vesicle (SUV) which may be smaller than 50 nm in diameter, and a large unilamellar vesicle (LUV) which may be between 50 nm and 500 nm in diameter.

LNPs of the invention are suitably characterized as microscopic vesicles having an interior aqua space sequestered from an outer medium by a membrane of one or more bilayers. Bilayer membranes of LNPs are typically formed by amphiphilic molecules, such as lipids of synthetic or natural origin that comprise spatially separated hydrophilic and hydrophobic domains. Bilayer membranes of the liposomes can also be formed by amphophilic polymers and surfactants (e.g., polymerosomes, niosomes, etc.).

Accordingly, in preferred embodiments, the at least one therapeutic RNA of the first component and/or the at least one antagonist (e.g. nucleic acid) of the second component, is complexed with one or more lipids thereby forming lipid nanoparticles (LNP).

LNPs typically comprise at least one cationic lipid and one or more excipient selected from neutral lipids, charged lipids, steroids and polymer conjugated lipids (e.g. PEGylated lipid). The at least one therapeutic RNA as defined herein/the at least one antagonist (e.g. nucleic acid) as defined herein may be encapsulated in the lipid portion of the LNP or an aqueous space enveloped by some or the entire lipid portion of the LNP. The at least one therapeutic RNA/the at least one antagonist (e.g. nucleic acid) or a portion thereof may also be associated and complexed with the LNP. An LNP may comprise any lipid capable of forming a particle to which the nucleic acids are attached, or in which the one or more nucleic acids are encapsulated. Preferably, the LNP comprises one or more cationic lipids, and one or more stabilizing lipids. Stabilizing lipids include neutral lipids and PEGylated lipids.

A cationic lipid of an LNP may be cationisable, i.e. it becomes protonated as the pH is lowered below the pK of the ionizable group of the lipid, but is progressively more neutral at higher pH values. At pH values below the pK, the lipid is then able to associate with negatively charged nucleic acids. In certain embodiments, the cationic lipid comprises a zwitterionic lipid that assumes a positive charge on pH decrease.

Such lipids include, but are not limited to, DSDMA, N,N-dioleyl-N,N-dimethylammonium chloride (DODAC), N,N-distearyl-N,N-dimethylammonium bromide (DDAB), 1,2-dioleoyltrimethyl ammonium propane chloride (DOTAP) (also known as N-(2,3-dioleoyloxy)propyl)-N,N,N-trimethylammonium chloride and 1,2-Dioleyloxy-3-trimethylaminopropane chloride salt), N-(1-(2,3-dioleyloxy)propyl)-N,N,N-trimethylammonium chloride (DOTMA), N,N-dimethyl-2,3-dioleyloxy)propylamine (DODMA), ckk-E12, ckk, 1,2-DiLinoleyloxy-N,N-dimethylaminopropane (DLinDMA), 1,2-Dilinolenyloxy-N,N-dimethylaminopropane (DLenDMA), 1,2-di-y-linolenyloxy-N,N-dimethylaminopropane (y-DLenDMA), 98N12-5, 1,2-Dilinoleylcarbamoyloxy-3- dimethylaminopropane (DLin-C-DAP), 1,2-Dilinoleyoxy-3-(dimethylamino)acetoxypropane (DLin-DAC), 1,2-Dilinoleyoxy-3-morpholinopropane (DLin-MA), 1,2-Dilinoleoyl-3-dimethylaminopropane (DLinDAP), 1,2-Dilinoleylthio-3-dimethylaminopropane (DLin-S- DMA), 1-Linoleoyl-2-linoleyloxy-3-dimethylaminopropane (DLin-2-DMAP), 1,2-Dilinoleyloxy-3-trimethylaminopropane chloride salt (DLin-TMA.CI), ICE (Imidazol-based), HGT5000, HGT5001, DMDMA, CLinDMA, CpLinDMA, DMOBA, DOcarbDAP, DLincarbDAP, DLinCDAP, KLin-K-DMA, DLin-K-XTC2-DMA, XTC (2,2-Dilinoleyl-4-dimethylaminoethyl-[1,3]-dioxolane) HGT4003, 1,2-Dilinoleoyl-3-trimethylaminopropane chloride salt (DLin-TAP.CI), 1,2-Dilinoleyloxy-3-(N- methylpiperazino)propane (DLin-MPZ), or 3-(N,N-Dilinoleylamino)-1,2-propanediol (DLinAP), 3-(N,N-Dioleylamino)-1,2-propanedio (DOAP), 1,2-Dilinoleyloxo-3-(2-N,N-dimethylamino)ethoxypropane (DLin-EG-DM A), 2,2-Dilinoleyl-4-dimethylaminomethyl-[1,3]-dioxolane (DLin-K-DMA) or analogs thereof, (3aR,5s,6aS)-N,N-dimethyl-2,2-di((9Z,12Z)-octadeca-9,12-dienyl)tetrahydro-3aH-cyclopenta[d][1,3]dioxol-5-amine, (6Z,9Z,28Z,31Z)-heptatriaconta-6,9,28,31-tetraen-19-yl-4-(dimethylamino)butanoate (MC3), ALNY-100 ((3aR,5s,6aS)- N,N-dimethyl-2,2-di((9Z,12Z)-octadeca-9,12-dienyl)tetrahydro-3aH-cyclopenta[d] [1,3]dioxol- 5-amine)), 1,1′-(2-(4-(2-((2-(bis(2-hydroxydodecyl)amino)ethyl)(2-hydroxydodecyl)amino)ethyl)piperazin-1-yl)ethylazanediyl)didodecan-2-ol (C12-200), 2,2-dilinoleyl-4-(2-dimethylaminoethyl)-[1,3]-dioxolane (DLin-K-C2-DMA), 2,2-dilinoleyl-4-dimethylaminomethyl-[1,3]-dioxolane (DLin-K-DMA), NC98-5 (4,7, 13-tris(3-oxo-3-(undecylamino)propyl)-NI,N 16-diundecyl-4,7, 10,13-tetraazahexadecane-1,16-diamide), (6Z,9Z,28Z,31Z)-heptatriaconta-6,9,28,31-tetraen-19-yl 4-(dimethylamino) butanoate (DLin-M-C3-DMA), 3-((6Z,9Z,28Z,31Z)-heptatriaconta-6,9,28,31-tetraen-19-yloxy)-N,N-dimethylpropan-1-amine (MC3 Ether), 4-((6Z,9Z,28Z,31Z)-heptatriaconta-6,9,28,31-tetraen-19-yloxy)-N,N-dimethylbutan-1-amine (MC4 Ether), LIPOFECTIN® (commercially available cationic liposomes comprising DOTMA and 1,2-dioleoyl-sn-3phosphoethanolamine (DOPE), from GIBCO/BRL, Grand Island, N.Y.); LIPOFECTAMINE® (commercially available cationic liposomes comprising N-(1-(2,3dioleyloxy)propyl)-N-(2-(sperminecarboxamido)ethyl)-N,N-dimethylammonium trifluoroacetate (DOSPA) and (DOPE), from GIBCO/BRL); and TRANSFECTAM® (commercially available cationic lipids comprising dioctadecylamidoglycyl carboxyspermine (DOGS) in ethanol from Promega Corp., Madison, Wis.) or any combination of any of the foregoing. Further suitable cationic lipids for use in the compositions and methods of the invention include those described in international patent publications WO2010/053572 (and particularly, CI 2-200 described at paragraph [00225]) and WO2012/170930, both of which are incorporated herein by reference, HGT4003, HGT5000, HGTS001, HGT5001, HGT5002 (see US20150140070A1).

In embodiments, the cationic lipid may be an amino lipid.

Representative amino lipids include, but are not limited to, 1,2-dilinoleyoxy-3-(dimethylamino)acetoxypropane (DLin-DAC), 1,2-dilinoleyoxy-3morpholinopropane (DLin-MA), 1,2-dilinoleoyl-3-dimethylaminopropane (DLinDAP), 1,2-dilinoleylthio-3-dimethylaminopropane (DLin-S-DMA), 1-linoleoyl-2-linoleyloxy-3dimethylaminopropane (DLin-2-DMAP), 1,2-dilinoleyloxy-3-trimethylaminopropane chloride salt (DLin-TMA.CI), 1,2-dilinoleoyl-3-trimethylaminopropane chloride salt (DLin-TAP.CI), 1,2-dilinoleyloxy-3-(N-methylpiperazino)propane (DLin-MPZ), 3-(N,Ndilinoleylamino)-1,2-propanediol (DLinAP), 3-(N,N-dioleylamino)-1,2-propanediol (DOAP), 1,2-dilinoleyloxo-3-(2-N,N-dimethylamino)ethoxypropane (DLin-EG-DMA), and 2,2-dilinoleyl-4-dimethylaminomethyl-[1,3]-dioxolane (DLin-K-DMA), 2,2-dilinoleyl-4-(2-dimethylaminoethyl)-[,3]-dioxolane (DLin-KC2-DMA); dilinoleyl-methyl-4-dimethylaminobutyrate (DLin-MC3-DMA); MC3 (US20100324120).

In one embodiment, the at least one therapeutic RNA as defined herein/the antagonist (e.g. nucleic acid) as defined herein, may be formulated in an aminoalcohol lipidoid. Aminoalcohol lipidoids which may be used in the present invention may be prepared by the methods described in U.S. Pat. No. 8,450,298, herein incorporated by reference in its entirety. Suitable (ionizable) lipids can also be the compounds as disclosed in Tables 1, 2 and 3 and as defined in claims 1-24 of published PCT patent application WO2017/075531A1, the specific disclosure hereby incorporated by reference.

In other embodiments, suitable lipids may be selected from published PCT patent application WO2015/074085A1 (i.e. ATX-001 to ATX-032 or the compounds as specified in claims 1-26), U.S. Appl. Nos. 61/905,724 and Ser. No. 15/614,499 or U.S. Pat. Nos. 9,593,077 and 9,567,296, hereby incorporated by reference.

In other embodiments, suitable cationic lipids may be selected from published PCT patent application WO2017/117530A1 (i.e. lipids 13, 14, 15, 16, 17, 18, 19, 20, or the compounds as specified in the claims), the specific disclosure hereby incorporated by reference.

In preferred embodiments, ionizable lipids/cationic lipids may also be selected from the lipids disclosed in published PCT patent application WO2018/078053A1 (i.e. lipids derived from formula I, II, and III of WO2018/078053A1, or lipids as specified in Claims 1 to 12 of WO2018/078053A1), the specific disclosure of WO2018/078053A1 relating thereto hereby incorporated by reference. In that context, lipids disclosed in Table 7 of WO2018/078053A1 (e.g. lipids derived from formula I-1 to I-41) and lipids disclosed in Table 8 of WO2018/078053A1 (e.g. lipids derived from formula II-1 to II-36) may be suitably used in the context of the invention. Accordingly, formula I-1 to formula I-41 and formula II-1 to formula II-36 of WO2018/078053A1, and the specific disclosure relating thereto, are herewith incorporated by reference.

In preferred embodiments, cationic lipids may be derived from formula Ill of published PCT patent application WO2018/078053A1. Accordingly, formula Ill of WO2018/078053A1, and the specific disclosure relating thereto, are herewith incorporated by reference.

In particularly preferred embodiments, the at least one therapeutic RNA as defined herein/the antagonist (e.g. nucleic acid) as defined herein is complexed with one or more lipids thereby forming LNPs, wherein the cationic lipid of the LNP is selected from structures III-1 to III-36 of Table 9 of published PCT patent application WO2018/078053A1. Accordingly, formula III-1 to III-36 of WO2018/078053A1, and the specific disclosure relating thereto, are herewith incorporated by reference.

In particularly preferred embodiment, the at least one therapeutic RNA as defined herein/the antagonist (e.g. nucleic acid) as defined herein is complexed with one or more lipids thereby forming LNPs, wherein the LNP comprises the following cationic lipid:

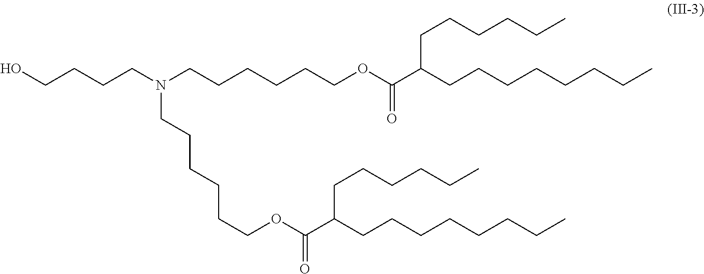 [SMILES: CCCCCCCCC(CCCCCC)C(=O)OCCCCCCN(CCCCO)CCCCCCOC(=O)C(CCCCCC)CCCCCCCC]

In certain embodiments, the cationic lipid (e.g. III-3) is present in the LNP in an amount from about 30 to about 95 mole percent, relative to the total lipid content of the LNP. If more than one cationic lipid is incorporated within the LNP, such percentages apply to the combined cationic lipids.

Other suitable (cationic or ionizable) lipids are disclosed in published patent applications WO2009/086558, WO2009/127060, WO2010/048536, WO2010/054406, WO2010/088537, WO2010/129709, WO2011/153493, WO 2013/063468, US2011/0256175, US2012/0128760, US2012/0027803, U.S. Pat. No. 8,158,601, WO2016/118724, WO2016/118725, WO2017/070613, WO2017/070620, WO2017/099823, WO2012/040184, WO2011/153120, WO2011/149733, WO2011/090965, WO2011/043913, WO2011/022460, WO2012/061259, WO2012/054365, WO2012/044638, WO2010/080724, WO2010/21865, WO2008/103276, WO2013/086373, WO2013/086354, and U.S. Pat. Nos. 7,893,302, 7,404,969, 8,283,333, 8,466,122 and 8,569,256 and US Patent Publication No. US2010/0036115, US2012/0202871, US2013/0064894, US2013/0129785, US2013/0150625, US20130178541, US2013/0225836, US2014/0039032 and WO2017/112865. In that context, the disclosures of WO2009/086558, WO2009/127060, WO2010/048536, WO2010/054406, WO2010/088537, WO2010/129709, WO2011/153493, WO 2013/063468, US2011/0256175, US2012/0128760, US2012/0027803, U.S. Pat. No. 8,158,601, WO2016/118724, WO2016/118725, WO2017/070613, WO2017/070620, WO2017/099823, WO2012/040184, WO2011/153120, WO2011/149733, WO2011/090965, WO2011/043913, WO2011/022460, WO2012/061259, WO2012/054365, WO2012/044638, WO2010/080724, WO2010/21865, WO2008/103276, WO2013/086373, WO2013/086354, U.S. Pat. Nos. 7,893,302, 7,404,969, 8,283,333, 8,466,122 and 8,569,256 and US Patent Publication No. US2010/0036115, US2012/0202871, US2013/0064894, US2013/0129785, US2013/0150625, US20130178541, US2013/0225836 and US2014/0039032 and WO2017/112865 specifically relating to (cationic) lipids suitable for LNPs are incorporated herewith by reference.

LNPs may comprise two or more (different) cationic lipids. The cationic lipids may be selected to contribute different advantageous properties. E.g., cationic lipids that differ in properties such as amine pKa, chemical stability, half-life in circulation, half-life in tissue, net accumulation in tissue, toxicity, or immune stimulation can be used in the LNP.

LNP in vivo characteristics and behavior can be modified by addition of a hydrophilic polymer coating, e.g. polyethylene glycol (PEG), to the LNP surface to confer steric stabilization. Furthermore, LNPs can be used for specific targeting by attaching ligands (e.g. antibodies, peptides, and carbohydrates) to its surface or to the terminal end of the attached PEG chains (e.g. via PEGylated lipids or PEGylated cholesterol).

In some embodiments, such PEG chains may be used to attach an antagonist of the invention.

In some embodiments, the LNPs comprise a polymer conjugated lipid. The term “polymer conjugated lipid” refers to a molecule comprising both a lipid portion and a polymer portion. An example of a polymer conjugated lipid is a PEGylated lipid. The term “PEGylated lipid” refers to a molecule comprising both a lipid portion and a polyethylene glycol portion. PEGylated lipids are known in the art and include 1-(monomethoxy-polyethyleneglycol)-2,3-dimyristoylglycerol (PEG-s- DMG) and the like.

In various embodiments, the LNP comprises a stabilizing-lipid which is a polyethylene glycol-lipid (PEGylated lipid). Suitable polyethylene glycol-lipids include PEG-modified phosphatidylethanolamine, PEG-modified phosphatidic acid, PEG-modified ceramides (e.g. PEG-CerC14 or PEG-CerC20), PEG-modified dialkylamines, PEG-modified diacylglycerols, PEG-modified dialkylglycerols. Representative polyethylene glycol-lipids include PEG-c-DOMG, PEG-c-DMA, PEG-s-DMG, PEG-DMG, PEG-DSG, PEG-DSPE, PEG-DOMG. In one embodiment, the polyethylene glycol-lipid is N-[(methoxy poly(ethylene glycol)2000)carbamyl]-1,2-dimyristyloxlpropyl-3-amine (PEG-c-DMA). In a preferred embodiment, the polyethylene glycol-lipid is PEG-2000-DMG. In one embodiment, the polyethylene glycol-lipid is PEG-c-DOMG). In other embodiments, the LNPs comprise a PEGylated diacylglycerol (PEG-DAG) such as 1-(monomethoxy-polyethyleneglycol)-2,3-dimyristoylglycerol (PEG-DMG), a PEGylated phosphatidylethanoloamine (PEG-PE), a PEG succinate diacylglycerol (PEG-S-DAG) such as 4-O-(2′,3′-di(tetradecanoyloxy)propyl-1-O-(w-methoxy(polyethoxy)ethyl)butanedioate (PEG-S-DMG), a PEGylated ceramide (PEG-cer), or a PEG dialkoxypropylcarbamate such as ω-methoxy(polyethoxy)ethyl-N-(2,3di(tetradecanoxy)propyl)carbamate or 2,3-di(tetradecanoxy)propyl-N-(ω-methoxy(polyethoxy)ethyl)carbamate.

In preferred embodiments, the PEGylated lipid that is preferably derived from formula (IV) of published PCT patent application WO2018/078053A1. Accordingly, PEGylated lipid derived from formula (IV) of published PCT patent application WO2018/078053A1, and the respective disclosure relating thereto, is herewith incorporated by reference.

In a particularly preferred embodiments, the therapeutic RNA of the first component and/or the at least one antagonist of the second component is complexed with one or more lipids thereby forming LNPs, wherein the LNP comprises a PEGylated lipid, wherein the PEG lipid is preferably derived from formula (IVa) of published PCT patent application WO2018/078053A1. Accordingly, PEGylated lipid derived from formula (IVa) of published PCT patent application WO2018/078053A1, and the respective disclosure relating thereto, is herewith incorporated by reference.

In a particularly preferred embodiment the PEG lipid is of formula (IVa)

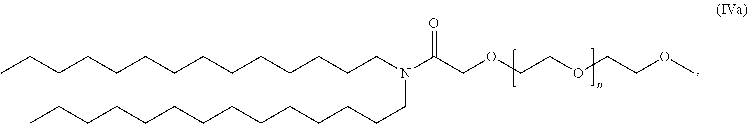

wherein n has a mean value ranging from 30 to 60, such as about 30±2, 32±2, 34±2, 36±2, 38±2, 40±2, 42±2, 44±2, 46±2, 48±2, 50±2, 52±2, 54±2, 56±2, 58±2, or 60±2. In a most preferred embodiment n is about 49.

Further examples of PEG-lipids suitable in that context are provided in US2015/0376115A1 and WO2015/199952, each of which is incorporated by reference in its entirety.

In some embodiments, LNPs include less than about 3, 2, or 1 mole percent of PEG or PEG-modified lipid, based on the total moles of lipid in the LNP. In further embodiments, LNPs comprise from about 0.1% to about 20% of the PEG-modified lipid on a molar basis. In preferred embodiments, LNPs comprise from about 1.0% to about 2.0% of the PEG-modified lipid on a molar basis. In various embodiments, the molar ratio of the cationic lipid to the PEGylated lipid ranges from about 100:1 to about 25:1.

In preferred embodiments, the LNP comprises one or more additional lipids which stabilize the formation of particles during their formation or during the manufacturing process (e.g. neutral lipid and/or one or more steroid or steroid analogue).

In preferred embodiments, the LNP comprises one or more neutral lipid and/or one or more steroid or steroid analogue.

Suitable stabilizing lipids include neutral lipids and anionic lipids. The term “neutral lipid” refers to any one of a number of lipid species that exist in either an uncharged or neutral zwitterionic form at physiological pH.

Representative neutral lipids include diacylphosphatidylcholines, diacylphosphatidylethanolamines, ceramides, sphingomyelins, dihydro sphingomyelins, cephalins, and cerebrosides.

In embodiments, the LNP comprises one or more neutral lipids, wherein the neutral lipid is selected from the group comprising distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), dioleoyl-phosphatidylethanolamine (DOPE), palmitoyloleoylphosphatidylcholine (POPC), palmitoyloleoyl-phosphatidylethanolamine (POPE) and dioleoyl-phosphatidylethanolamine 4-(N-maleimidomethyl)-cyclohexane-1carboxylate (DOPE-mal), dipalmitoyl phosphatidyl ethanolamine (DPPE), dimyristoylphosphoethanolamine (DMPE), distearoyl-phosphatidylethanolamine (DSPE), 16-O-monomethyl PE, 16-O-dimethyl PE, 18-1-trans PE, 1-stearioyl-2-oleoylphosphatidyethanol amine (SOPE), and 1,2-dielaidoyl-sn-glycero-3-phophoethanolamine (transDOPE), or mixtures thereof.

In some embodiments, the LNPs comprise a neutral lipid selected from DSPC, DPPC, DMPC, DOPC, POPC, DOPE and SM. In various embodiments, the molar ratio of the cationic lipid to the neutral lipid ranges from about 2:1 to about 8:1. In preferred embodiments, the neutral lipid is 1,2-distearoyl-sn-glycero-3-phosphocholine (DSPC). The molar ratio of the cationic lipid to DSPC may be in the range from about 2:1 to 8:1. In preferred embodiments, the steroid is cholesterol. The molar ratio of the cationic lipid to cholesterol may be in the range from about 2:1 to 1:1. In some embodiments, the cholesterol may be PEGylated.

In particularly preferred embodiments, the lipid is lipid compound is or is derived from formula Ill, preferably 111-3, the neutral lipid is DSPC, the steroid is cholesterol, and the PEGylated lipid is the compound of formula (IVa).

In a preferred embodiments, the liposomes, lipid nanoparticles, lipoplexes, and/or nanoliposomes preferably comprises or consist of (i) at least one cationic lipid; (ii) at least one neutral lipid; (iii) at least one steroid or steroid analogue; and (iv) at least one aggregation reducing-lipid, wherein, preferably, (i) to (iv) are in a molar ratio of about 20-60% cationic lipid, 5-25% neutral lipid, 25-55% sterol, and 0.5-15% PEG-lipid.

In specific embodiments, the at least one therapeutic RNA of the first component and/or the at least one antagonist of the second component (e.g. nucleic acid) is complexed with one or more lipids thereby forming LNPs, wherein the LNP comprises

(i) at least one cationic lipid as defined herein, preferably lipid III-3;

(ii) at least one neutral lipid as defined herein, preferably 1,2-distearoyl-sn-glycero-3-phosphocholine (DSPC);

(iii) at least one steroid or steroid analogue as defined herein, preferably cholesterol; and

(iv) at least one PEG-lipid as defined herein, e.g. PEG-DMG or PEG-cDMA, preferably a PEGylated lipid of formula (IVa), wherein, preferably, (i) to (iv) are in a molar ratio of about 20-60% cationic lipid; 5-25% neutral lipid; 25-55% sterol; 0.5-15% PEG-lipid.

In a particular preferred embodiment, the at least one therapeutic RNA of the first component and/or the at least one antagonist of the second component (e.g. nucleic acid) is complexed with one or more lipids thereby forming LNPs, wherein LNPs have a molar ratio of approximately 50:10:38.5:1.5, preferably 47.5:10:40.8:1.7 or more preferably 47.4:10:40.9:1.7 (i.e. proportion (mol %) of cationic lipid (preferably lipid 111-3), DSPC, cholesterol and PEG-lipid ((preferably PEG-lipid of formula (IVa) with n=49); solubilized in ethanol).

In various embodiments, the LNPs as defined herein have a mean diameter of from about 50 nm to about 200 nm, from about 50 nm to about 150 nm, or from about 50 nm to about 100 nm. As used herein, the mean diameter may be represented by the z-average as determined by dynamic light scattering as commonly known in the art. The polydispersity index (PDI) of the LNPs is suitably in the range of 0.1 to 0.5. In a particular embodiment, a PDI is below 0.2. Typically, the PDI is determined by dynamic light scattering as commonly known in the art.

In preferred embodiments, administration of the combination, preferably administration of first component and the second component is essentially simultaneous.

“Simultaneous” in that context has to be understood as that administration of the first and the second component of the combination may occur simultaneously and not in a timely staggered manner. Said simultaneous administration may be either at the same site of administration/administration route or at different sites of administration/administration route, as further outlined below.

In other preferred embodiments, administration of the combination, preferably administration of first component and the second component is sequential.

“Sequential” in that context has to be understood as that administration of the first and the second component of the combination may occur in a timely staggered manner and not simultaneously. Said “sequential” administration may be either at the same site of administration or at different sites of administration, as further outlined below.

In preferred embodiments, administration of the combination, that is administration of the first component and/or the second component (sequential or simultaneous) is performed more than once, for example once or more than once a day, once or more than once a week, once or more than once a month. Advantageously, the combination of the invention is suitable for repetitive administration, e.g. for chronic administration.

The combination may be administered orally, parenterally, by inhalation spray, topically, rectally, nasally, buccally, vaginally or via an implanted reservoir. The term parenteral, as used herein, includes subcutaneous, intravenous, intramuscular, intra-articular, intra-synovial, intrasternal, intrathecal, intrahepatic, intralesional, intracranial, transdermal, intradermal, intrapulmonal, intraperitoneal, intracardial, intraarterial, intraocular, intravitreal, subretinal, intratuomoral.

In particularly preferred embodiments, administration of the combination, in particular administration of the first component and/or the second component (sequential or simultaneous), is performed intravenously. In particular embodiments, the combination is administered intravenously as a chronic treatment (e.g. more than once, for example once or more than once a day, once or more than once a week, once or more than once a month).

In a particularly preferred embodiment, the combination is characterized by the following features:
(I) at least one first component as defined herein, preferably an mRNA encoding a therapeutic peptide or protein, e.g. an antibody, an enzyme, an antigen, wherein, optionally, said mRNA does not comprise modified nucleotides, wherein said mRNA does comprise a Cap1 structure (preferably obtainable by co-transcriptional capping), wherein said first component is formulated in a lipid nanoparticle or in a polyethylene glycol/peptide polymer.
(II) at least one second component as defined herein, preferably a single stranded RNA oligonucleotide comprising at least one 2-O-methylated RNA nucleotide, preferably comprising a nucleic acid sequence according to formula I, wherein said second component is formulated in a lipid nanoparticle or in a polyethylene glycol/peptide polymer.

In some embodiments, administration of the combination to a cell, tissue, or organism results in an increased expression for example as compared to administration of the corresponding first component alone.

In particular, the reduction of the (innate) immune stimulation promotes the translation of the first component.

Composition

In a second aspect, the present invention provides a composition comprising the first component as defined herein and the second component as defined herein.

In preferred embodiments, the pharmaceutical composition comprises or consists of

(i) at least one therapeutic RNA;

(ii) at least one antagonist of at least one RNA sensing pattern recognition receptor, and optionally, at least one pharmaceutically acceptable carrier.

Preferably, the at least one therapeutic RNA is as described in the context of the combination as “the first component”, and the at least one antagonist is as described in the context of the combination as “the second component”. Accordingly, embodiments described above (in the context of the first aspect) relating to the first component of the combination are also applicable to the at least one therapeutic RNA of the composition.

Additionally, embodiments described above (in the context of the first aspect) relating to the second component of the combination are also applicable to the at least one antagonist of at least one RNA sensing pattern recognition receptor of the composition.

In preferred embodiments, the pharmaceutical composition of the second aspect consists or comprises a combination as defined in the context of the first aspect, and optionally at least one pharmaceutically acceptable carrier.

The term “pharmaceutically acceptable carrier” or “pharmaceutically acceptable excipient” as used herein preferably includes the liquid or non-liquid basis of the first and/or the second component. If the first and/or the second component are provided in liquid form, the carrier may be water, e.g. pyrogen-free water; isotonic saline or buffered (aqueous) solutions, e.g. phosphate, citrate etc. buffered solutions. Water or preferably a buffer, more preferably an aqueous buffer, may be used, containing a sodium salt, preferably at least 50 mM of a sodium salt, a calcium salt, preferably at least 0.01 mM of a calcium salt, and optionally a potassium salt, preferably at least 3 mM of a potassium salt. According to preferred embodiments, the sodium, calcium and, optionally, potassium salts may occur in the form of their halogenides, e.g. chlorides, iodides, or bromides, in the form of their hydroxides, carbonates, hydrogen carbonates, or sulphates, etc. Examples of sodium salts include NaCl, NaI, NaBr, Na₂CO₃, NaHCO₃, Na₂SO₄, examples of the optional potassium salts include KCl, KI, KBr, K₂CO₃, KHCO₃, K₂SO₄, and examples of calcium salts include CaCl₂, CaI₂, CaBr₂, CaCO₃, CaSO₄, Ca(OH)₂.

Notably, a suitable pharmaceutically acceptable carrier refers to a substance that does not interfere with the effectiveness of the first and or second component, the combination or the composition as defined herein, and that is compatible with a biological system such as a cell, cell culture, tissue, or organism.

Further advantageous embodiments and features of the pharmaceutical composition of the invention are described below. Notably, embodiments and features described in the context of the pharmaceutical composition may likewise be applicable to the combination of the first aspect and/or the kit or kit of parts of the third aspect.

Accordingly, the pharmaceutical composition comprises or consists of

(i) at least one therapeutic RNA, wherein at least one therapeutic RNA is the “first component” as defined in the context of the first aspect;

(ii) at least one antagonist of at least one RNA sensing pattern recognition receptor, wherein at least one antagonist is the “second component” as defined in the context of the first aspect; and optionally, at least one pharmaceutically acceptable carrier, preferably a pharmaceutically acceptable carrier as defined above.

In preferred embodiments, the pharmaceutical composition comprises or consists of

(i) at least one therapeutic RNA, wherein at least one therapeutic RNA is a “first component”;

(ii) at least one antagonist of at least one RNA sensing pattern recognition receptor, wherein at least one antagonist is the “second component”, preferably a nucleic acid;

The composition suitably comprises a safe and effective amount of the therapeutic RNA as specified herein. As used herein, “safe and effective amount” means an amount of the therapeutic RNA, preferably the mRNA, sufficient to result in expression and/or activity of the encoded protein after administration. At the same time, a “safe and effective amount” is small enough to avoid serious side-effects caused by administration of said therapeutic RNA.

Further, the composition suitably comprises a safe and effective amount of the at least one antagonist of at least one RNA sensing pattern recognition receptor, preferably the nucleic acid as specified herein. As used herein, “safe and effective amount” means an amount of antagonist, preferably the nucleic acid, sufficient to result in antagonizing of at least one RNA sensing pattern recognition receptor after administration. At the same time, a “safe and effective amount” is small enough to avoid serious side-effects caused by administration of said antagonist.

A “safe and effective amount” of the first and the second component of the composition will furthermore vary in connection with the particular condition to be treated and also with the age and physical condition of the patient to be treated, the severity of the condition, the duration of the treatment, the nature of the accompanying therapy, of the particular pharmaceutically acceptable carrier used etc. Moreover, the “safe and effective amount” of the first and the second component as described herein may depend from application route (e.g. intravenous, intramuscular), application device (needle injection, injection device), and/or complexation/formulation (e.g. RNA in association with a polymeric carrier or LNP). Moreover, the “safe and effective amount” of the composition may depend on the condition of the treated subject (infant, immunocompromised human subject etc.).

In the context of the invention, a “composition” refers to any type of composition in which the specified ingredients (e.g. first component as defined herein, e.g. mRNA and/or second component as defined herein, e.g. nucleic acid), may be incorporated, optionally along with any further constituents, usually with at least one pharmaceutically acceptable carrier or excipient. The composition may be a dry composition such as a powder or granules, or a solid unit such as a lyophilized form. The composition may be in liquid form, and each constituent may be independently incorporated in dissolved or dispersed (e.g. suspended or emulsified) form.

The term “subject”, “patient” or “individual” as used herein generally includes humans and non-human animals and preferably mammals, including chimeric and transgenic animals and disease models. Subjects to which administration of the compositions, preferably the pharmaceutical composition, is contemplated include, but are not limited to, humans and/or other primates; mammals, including commercially relevant mammals such as cattle, pigs, horses, sheep, cats, dogs; and/or birds, including commercially relevant birds such as poultry, chickens, ducks, geese, and/or turkeys. Preferably, the term “subject” refers to a non-human primate or a human, most preferably a human.

In preferred embodiments, a “subject in need of treatment”, or a “subject in need thereof” in the context of the invention is a human subject.

In embodiments, the composition may comprise a plurality or at least more than one of therapeutic RNA species, as defined above, wherein each therapeutic RNA species, e.g. each mRNA species, may encode a different therapeutic peptide or protein as defined.

In embodiments, the composition comprises more than one or a plurality, e.g. 2, 3, 4, 5, 6, 7, 8, 9, 10, 11, 12, 13, 14, 15 of different therapeutic RNA species of the first component as defined above.

The term “RNA species” as used herein is not intended to refer to only one single molecule. The term “RNA species” has to be understood as an ensemble of essentially identical RNA molecules, wherein each of the RNA molecules of the RNA ensemble, in other words each of the molecules of the RNA species, encodes the same therapeutic protein (in embodiments where the therapeutic RNA is a coding RNA), having essentially the same nucleic acid sequence. However, the RNA molecules of the RNA ensemble may differ in length or quality which may be caused by the enzymatic or chemical manufacturing process.

In embodiments, the composition comprises more than one or a plurality of different therapeutic RNA species of the first component, wherein the more than one or a plurality of different therapeutic RNA species is selected from coding RNA species each encoding a different protein.

In embodiments, the composition comprises more than one or a plurality of different therapeutic RNA species of the first component, wherein at least one of the more than one or a plurality of different therapeutic RNA species is selected from a coding RNA species (e.g., an mRNA encoding a CRISPR associated endonuclease), and at least one is selected from a non-coding RNA species (e.g., a guide RNA).

In embodiments, the composition comprises more than one or a plurality, e.g. 2, 3, 4, 5, 6, 7, 8, 9, 10, 11, 12, 13, 14, 15 of different antagonists of the second component, preferably nucleic acid species, as defined above.

The term “nucleic acid species” as used herein is not intended to refer to only one single nucleic acid molecule.

The term “nucleic acid species” in the context of the second component has to be understood as an ensemble of essentially identical nucleic acid molecules, wherein each of the nucleic acid molecules of such an ensemble has essentially the same nucleic acid sequence.

In preferred embodiments, the composition comprises the therapeutic RNA of the first component, preferably an mRNA, and the antagonist of the second component, preferably a nucleic acid, wherein said first component and/or said second component are complexed or associated with or at least partially complexed or partially associated with one or more cationic or polycationic compound, preferably cationic or polycationic polymer, cationic or polycationic polysaccharide, cationic or polycationic lipid, cationic or polycationic protein, or cationic or polycationic peptide, or any combinations thereof. Complexation/association (“formulation”) to carriers as defined herein facilitates the uptake of the therapeutic RNA and/or the antagonist into cells.

The term “cationic or polycationic compound” as used herein will be recognized and understood by the person of ordinary skill in the art, and is for example intended to refer to a charged molecule, which is positively charged at a pH value ranging from about 1 to 9, at a pH value ranging from about 3 to 8, at a pH value ranging from about 4 to 8, at a pH value ranging from about 5 to 8, more preferably at a pH value ranging from about 6 to 8, even more preferably at a pH value ranging from about 7 to 8, most preferably at a physiological pH, e.g. ranging from about 7.2 to about 7.5. Accordingly, a cationic component, e.g. a cationic peptide, cationic protein, cationic polymer, cationic polysaccharide, cationic lipid (including lipidoids) may be any positively charged compound or polymer which is positively charged under physiological conditions. A “cationic or polycationic peptide or protein” may contain at least one positively charged amino acid, or more than one positively charged amino acid, e.g. selected from Arg, His, Lys or Orn. Accordingly, “polycationic” components are also within the scope exhibiting more than one positive charge under the given conditions.

Cationic or polycationic compounds, being particularly preferred in this context may be selected from the following list of cationic or polycationic peptides or proteins of fragments thereof: protamine, nucleoline, spermine or spermidine, or other cationic peptides or proteins, such as poly-L-lysine (PLL), poly-arginine, basic polypeptides, cell penetrating peptides (CPPs), including HIV-binding peptides, HIV-1 Tat (HIV), Tat-derived peptides, Penetratin, VP22 derived or analog peptides, HSV VP22 (Herpes simplex), MAP, KALA or protein transduction domains (PTDs), PpT620, prolin-rich peptides, arginine-rich peptides, lysine-rich peptides, MPG-peptide(s), Pep-1, L-oligomers, Calcitonin peptide(s), Antennapedia-derived peptides, pAntp, pisl, FGF, Lactoferrin, Transportan, Buforin-2, Bac715-24, SynB, SynB(1), pVEC, hCT-derived peptides, SAP, or histones.

Further preferred cationic or polycationic compounds, which can be used as transfection or complexation agent may include cationic polysaccharides, for example chitosan, polybrene etc.; cationic lipids, e.g. DOTMA, DMRIE, di-C14-amidine, DOTIM, SAINT, DC-Chol, BGTC, CTAP, DOPC, DODAP, DOPE: Dioleyl phosphatidylethanol-amine, DOSPA, DODAB, DOIC, DMEPC, DOGS, DIMRI, DOTAP, DC-6-14, CLIP1, CLIP6, CLIP9, oligofectamine; or cationic or polycationic polymers, e.g. modified polyaminoacids, such as beta-aminoacid-polymers or reversed polyamides, etc., modified polyethylenes, such as PVP etc., modified acrylates, such as pDMAEMA etc., modified amidoamines such as pAMAM etc., modified polybetaaminoester (PBAE), such as diamine end modified 1,4 butanediol diacrylate-co-5-amino-1-pentanol polymers, etc., dendrimers, such as polypropylamine dendrimers or pAMAM based dendrimers, etc., polyimine(s), such as PEI, poly(propyleneimine), etc., polyallylamine, sugar backbone based polymers, such as cyclodextrin based polymers, dextran based polymers, etc., silan backbone based polymers, such as PMOXA-PDMS copolymers, etc., blockpolymers consisting of a combination of one or more cationic blocks (e.g. selected from a cationic polymer as mentioned above) and of one or more hydrophilic or hydrophobic blocks (e.g. polyethyleneglycole); etc.

In embodiments, the composition comprising the at least one therapeutic RNA and the at least one antagonist are formulated separately. Accordingly, the first component (as defined in the first aspect) and the second component (as defined in the first aspect) may be formulated (complexed/associated) as separate entities. The formulation/complexation of the components may be the same (e.g. both components complexed in polymeric carriers) or may be different (e.g. one component encapsulated in LNPs, the other component complexed in polymeric particle).

In embodiments, the composition comprising the at least one therapeutic RNA and the at least one antagonist are co-formulated. Accordingly, the first component (as defined in the first aspect) and the second component (as defined in the first aspect) are formulated (complexed/associated) as one entities. In these embodiments, the formulation/complexation of the components is the same (e.g. both components in an LNP).

In preferred embodiments, the at least one therapeutic RNA and the at least one antagonist are co-formulated to increase the probability that they are both present in one particle to ensure that the at least one therapeutic RNA and the at least one antagonist are up taken by the same cell.

In that context, suitable cationic or polycationic compounds for formulation may be selected from any one as defined in the context of the first aspect. The first and second component of the composition may be complexed or associated with the same cationic or polycationic compound, or with different cationic or polycationic compounds. In preferred embodiments, the first and second component of the composition may be complexed or associated within the same cationic or polycationic compound (that is “co-formulated”). In other embodiments, the first and second component of the composition may be complexed or associated within different cationic or polycationic compound.

In preferred embodiments of the composition, the polymeric carrier (of the first and/or second component) is a peptide polymer, preferably a polyethylene glycol/peptide polymer as defined above, and a lipid, preferably a lipidoid. In preferred embodiments, the first and second component of the composition may be complexed or associated within the same polymeric compound (that is “co-formulated”). In other embodiments, the first and second component of the composition may be complexed or associated within different polymeric compound (that is “formulated separately”).

In preferred embodiments of the composition, the at least one therapeutic RNA of the first compound, preferably the mRNA is complexed, partially complexed, encapsulated, partially encapsulated, or associated with one or more lipids (e.g. cationic lipids and/or neutral lipids), thereby forming liposomes, lipid nanoparticles (LNPs), lipoplexes, and/or nanoliposomes and/or the at least one antagonist of the second compound, preferably the nucleic acid, is complexed, partially complexed, encapsulated, partially encapsulated, or associated with one or more lipids (e.g. cationic lipids and/or neutral lipids), thereby forming liposomes, lipid nanoparticles (LNPs), lipoplexes, and/or nanoliposomes.

Suitable liposomes/lipid nanoparticles may be derived from the disclosure provided in the context of the first aspect.

The first and second component of the composition may be complexed or associated within the same lipid nanoparticles, or with different lipid nanoparticles. In preferred embodiments, the first and second component of the composition may be complexed or associated within the same lipid nanoparticle (that is “co-formulated”). As mentioned above, co-formulation increase the probability that they are both present in one particle to ensure that the at least one therapeutic RNA and the at least one antagonist are up taken by the same cell.

In preferred embodiments of the composition, the at least one therapeutic RNA of the first compound is an mRNA, and the at least one antagonist of the second compound is an RNA oligonucleotide, co-formulated in liposomes/lipid nanoparticles as defined herein.

In embodiments of the composition (or combination) the molar ratio of the at least one antagonist of the second component, preferably the nucleic acid as defined herein, to the at least one therapeutic RNA of the first component ranges from about 1:1 to about 100:1, or ranges from about 20:1 to about 80:1.

In preferred embodiments of the composition (or combination), the molar ratio of the at least one antagonist of the second component, preferably the nucleic acid as defined herein, to the at least one therapeutic RNA of the first component ranges from about 200:1 to about 1:1, or from about 100:1 to about 1:1, or from about 90:1 to about 1:1, or from about 80:1 to about 1:1, or from about 70:1 to about 1:1, or from about 60:1 to about 1:1, or from about 50:1 to about 1:1, or from about 40:1 to about 1:1, or from about 30:1 to about 1:1, or from about 20:1 to about 1:1, or from about 10:1 to about 1:1, or from about 5:1 to about 1:1, or from about 4:1 to about 1:1, or from about 3:1 to about 1:1, or from about 2:1 to about 1:1 or ranges from about 1:1 to about 1:200, or from about 1:1 to about 1:100, or from about 1:1 to about 1:90, or from about 1:1 to about 1:80, or from about 1:1 to about 1:70, or from about 1:1 to about 1:60, or from about 1:1 to about 1:50, or from about 1:1 to about 1:40, or from about 1:1 to about 1:30, or ranges from about 1:1 to about 1:20 or ranges from about 1:1 to about 1:10, or ranges from about 1:1 to about 1:5, or ranges from about 1:1 to about 1:4, or ranges from about 1:1 to about 1:3, or ranges from about 1:1 to about 1:2.

In specific embodiments of the composition, the molar ratio of the at least one antagonist of the second component, preferably the nucleic acid as defined herein, to the at least one therapeutic RNA of the first component is about 1:1, 2:1, 3:1, 4:1, 5:1, 6:1, 7:1, 8:1, 9:1, 10:1, 11:1, 12:1, 13:1, 14:1, 15:1, 16:1, 17:1, 18:1, 19:1, 20:1, 30:1, 40:1, 50:1, 60:1, 70:1, 80:1, 90:1, 100:1 or 1:2, 1:3, 1:4, 1:5, 1:6, 1:7, 1:8, 1:9, 1:10, 1:11, 1:12, 1:13, 1:14, 1:15, 1:16, 1:17, 1:18, 1:19, 1:20, 1:30, 1:40, 1:50; 1:59, 1:60, 1:70, 1:80, 1:90, 1:100.

In embodiments of the composition (or combination) the weight to weight ratio of the at least one antagonist of the second component, preferably the nucleic acid as defined herein, to the at least one therapeutic RNA of the first component ranges from about 1:1 to about 1:30, or ranges from about 1:2 to about 1:20.

In preferred embodiments of the composition (or combination), the weight to weight ratio of the at least one antagonist of the second component, preferably the nucleic acid as defined herein, to the at least one therapeutic RNA of the first component ranges from about 1:1 to about 1:20, or from about 1:1 to about 1:15, or from about 1:1 to about 1:10, or from about 1:1 to about 1:9, or from about 1:1 to about 1:8, or from about 1:1 to about 1:7, or from about 1:1 to about 1:6, or from about 1:1 to about 1:5, or from about 1:1 to about 1:4, or from about 1:1 to about 1:3, or from about 1:1 to about 1:2, or ranges from about 10:1 to about 1:1, or from about 9:1 to about 1:1, or from about 8:1 to about 1:1, or from about 7:1 to about 1:1, or from about 6:1 to about 1:1, or from about 5:1 to about 1:1, or from about 4:1 to about 1:1, or from about 3:1 to about 1:1, or from about 2:1 to about 1:1.

In specific embodiments of the composition, the weight to weight ratio of the at least one antagonist of the second component, preferably the nucleic acid as defined herein, to the at least one therapeutic RNA of the first component is about 1:1, 1:2, 1:3, 1:4, 1:5, 1:6, 1:7, 1:8, 1:9, 1:10, 1:11, 1:12, 1:13, 1:14, 1:15, 1:16, 1:17, 1:18, 1:19, 1:20, 1:30, 1:40, 1:50, or 2:1, 3:1, 4:1, 5:1, 6:1, 7:1, 8:1, 9:1, 10:1, 11:1, 12:1, 13:1, 14:1, 15:1, 16:1, 17:1, 18:1, 19:1, 20:1, 30:1, 40:1, 50:1.

Particularly preferred are weight to weight ratio of the at least one antagonist of the second component, preferably the nucleic acid as defined herein, to the at least one therapeutic RNA of the first component ranging from about 1:2 to about 1:20, specifically about 1:5, 1:10, or 1:15.

Accordingly, the percentage of mass (% mass of total nucleic acid) of the at least one antagonist, in particular of the nucleic acid of the second component in the composition or combination comprises about 40%, 35%, 30%, 25%, 24%, 23%, 22%, 21%, 20%, 19%, 18%, 17%, 16%, 15%, 14%, 13%, 12%, 11%, 10%, 9%, 8%, 7%, 6%, 5%, 4%, 3%, 2%, or 1%.

In embodiments of the composition (or combination), the therapeutic RNA of the first compound is provided in an amount of about 20 ng to about 1000 μg, about 0.2 μg to about 1000 μg, about 0.2 μg to about 900 μg, about 0.2 μg to about 800 μg, about 0.2 μg to about 700 μg, about 0.2 μg to about 600 μg, about 0.2 μg to about 500 μg, about 0.2 μg to about 400 μg, about 0.2 μg to about 300 μg, about 0.2 μg to about 100 μg, about 0.2 μg to about 100 μg, about 0.2 μg to about 80 μg, about 0.2 μg to about 60 μg, about 0.2 μg to about 40 μg, about 0.2 μg to about 20 μg, about 0.2 μg to about 10 μg, about 0.2 μg to about 5 μg, about 0.2 μg to about 2 μg, specifically, in an amount of about 0.2 μg, about 0.4 μg, about 0.6 μg, about 0.8 μg, about 1 μg, about 1.2 μg, about 1.4 μg, about 1.6 μg, about 1.8 μg, about 2 μg, about 3 μg, about 4 μg, about 5 μg, about 6 μg, about 7 μg, about 8 μg, about 9 μg, about 10 μg, about 11 μg, about 12 μg, about 14 μg, about 16 μg, about 18 μg, about 20 μg, about 40 μg, about 60 μg, about 80 μg, about 100 μg.

In embodiments of the composition (or combination), the therapeutic RNA of the first compound is provided in an amount of about 20 μg to about 200 mg, about 0.2 mg to about 200 mg, about 0.2 mg to about 180 mg, about 0.2 mg to about 160 mg, about 0.2 mg to about 140 mg, about 0.2 mg to about 120 mg, about 0.2 mg to about 100 mg, 0.2 mg to about 80 mg, about 0.2 mg to about 60 mg, about 0.2 mg to about 50 mg, about 0.2 mg to about 40 mg, about 0.2 mg to about 30 mg, about 0.2 mg to about 20 mg, about 0.2 mg to about 10 mg, about 1 mg to about 10 mg, specifically, in an amount of about 0.2 mg, about 0.4 mg, about 0.6 mg, about 0.8 mg, about 1 mg, about 1.2 mg, about 1.4 mg, about 1.6 mg, about 1.8 mg, about 2 mg, about 3 mg, about 4 mg, about 5 mg, about 6 mg, about 7 mg, about 8 mg, about 9 mg, about 10 mg, about 11 mg, about 12 mg, about 14 mg, about 16 mg, about 18 mg, about 20 mg, about 40 mg, about 60 mg, about 80 mg, about 100 mg.

In embodiments of the composition (or combination), the antagonist of the second compound, preferably the nucleic acid is provided in an amount of about 1 ng to about 50 μg, 2 ng to about 100 μg, about 2 ng to about 80 μg, 2 ng to about 60 μg, about 2 ng to about 40 μg, about 2 ng to about 20 μg, about 2 ng to about 10 μg, about 2 ng to about 5 μg, about 2 ng to about 2 μg, specifically, in an amount of about 2 ng, about 4 ng, about 6 ng, about 8 ng, about 10 ng, about 12 ng, about 14 ng, about 16 ng, about 18 ng, about 20 ng, about 30 ng, about 40 ng, about 50 ng, about 60 ng, about 70 ng, about 80 ng, about 90 ng, about 100 ng, about 11 Ong, about 140 ng, about 160 ng, about 180 ng, about 200 ng, about 400 ng, about 600 ng, about 800 ng, about 1000 ng.

In embodiments of the composition (or combination), the antagonist of the second compound, preferably the nucleic acid is provided in an amount of about 2 μg to about 20 mg, about 20 μg to about 20 mg, about 20 μg to about 18 mg, about 20 μg to about 16 mg, about 20 μg to about 14 mg, about 20 μg to about 12 mg, about 20 μg to about 10 mg, about 20 μg to about 8 mg, about 20 μg to about 6 mg, about 20 μg to about 4 mg, about 20 μg to about 2 mg, about 20 μg to about 1 mg, specifically, in an amount of about 2 μg, about 4 μg, about 6 μg, about 8 μg, about 10 μg, about 12 μg, about 14 μg, about 16 μg, about 18 μg, about 20 μg, about 30 μg, about 40 μg, about 50 μg, about 60 μg, about 70 μg, about 80 μg, about 90 μg, about 100 μg, about 110 μg, about 140 μg, about 160 μg, about 180 μg, about 200 μg, about 400 μg, about 600 μg, about 800 μg, about 1000 μg.

In preferred embodiments, the composition comprises about 20 ng to about 100 μg therapeutic RNA of the first compound, preferably mRNA as defined herein, and about 0.2 ng to about 10 μg antagonist of the second compound, preferably the nucleic acid antagonist as defined herein.

In other preferred embodiments, the composition comprises about 200 μg to about 200 mg therapeutic RNA of the first compound, preferably mRNA as defined herein, and about 20 μg to about 20 mg antagonist of the second compound, preferably the nucleic acid antagonist as defined herein.

In preferred embodiments the composition comprising the first and the second component is administered in Ringer or Ringer-Lactate solution.

In preferred embodiments, administration of the composition to a cell, tissue, or organism results in increased or prolonged or at least a comparable activity of the therapeutic RNA of the first component (comprised in said composition) as compared to administration of a corresponding first component as only.

The meaning of the term “activity” in that context depends on the therapeutic modality of the therapeutic RNA of the first component. Accordingly, “activity” is closely linked to the therapeutic effect of the therapeutic RNA of the first component. In embodiments where the therapeutic RNA is a coding RNA, “activity” has to be understood as expression, e.g. protein expression that occurs after administration to a cell, tissue, or organism, wherein the protein is provided by the cds of the administered coding RNA (e.g., the mRNA). In embodiments where the therapeutic RNA is a coding RNA encoding an antigen, “activity” has to be understood as expression, e.g. protein expression that occurs after administration to a cell, tissue, or organism, wherein the protein is provided by the cds of the administered coding RNA (e.g., the mRNA) and/or to the induction of antigen-specific immune responses (e.g. B-cell responses and/or T-cell responses).

In particularly preferred embodiments, administration of the composition to a cell, tissue, or organism, results in increased or prolonged activity of the therapeutic RNA of the first component (comprised in the composition) as compared to administration of a corresponding first component as control.

In other particularly preferred embodiments, administration of the composition to a cell, tissue, or organism results in increased or prolonged activity of the therapeutic RNA (comprising non-modified nucleotides) of the first component comprised in said composition as compared to administration of a corresponding first component as control (wherein the RNA comprises modified nucleotides and has the same RNA sequence).

Accordingly, in preferred embodiments of the composition, activity of the therapeutic RNA (or the corresponding controls) is expression, preferably protein expression, preferably protein expression of a coding therapeutic RNA, e.g. therapeutic mRNA. Expression may be determined as defined in the context of the first aspect.

In preferred embodiments, administration of the composition to a cell, tissue, or organism results in a reduced (innate) immune stimulation as compared to administration of the therapeutic RNA or the first component as a control.

In further preferred embodiments, administration of the composition to a cell, tissue, or organism results in essentially the same or at least a comparable (innate) immune stimulation as compared to administration of a control RNA comprising modified nucleotides (e.g. as defined herein) and having the same RNA sequence.

Preferably, reduced immune stimulation of the composition is a reduced level of at least one cytokine selected from Rantes, MIP-1 alpha, MIP-1 beta, McP1, TNFalpha, IFNgamma, IFNalpha, IFNbeta, IL-12, IL-6, or IL-8. Cytokine levels may be determined as defined in the context of the first aspect.

In preferred embodiments, administration the composition is performed more than once, for example once or more than once a day, once or more than once a week, once or more than once a month. Advantageously, the composition of the invention is suitable for repetitive administration, e.g. for chronic administration.

The composition may be administered orally, parenterally, by inhalation spray, topically, rectally, nasally, buccally, vaginally or via an implanted reservoir. The term parenteral, as used herein, includes subcutaneous, intravenous, intramuscular, intra-articular, intra-synovial, intrasternal, intrathecal, intrahepatic, intralesional, intracranial, transdermal, intradermal, intrapulmonal, intraperitoneal, intracardial, intraarterial, intraocular, intravitreal, subretinal, intratuomoral.

In particularly preferred embodiments, administration of the composition is performed intravenously. In particular embodiments, the composition is administered intravenously as a chronic treatment (e.g. more than once, for example once or more than once a day, once or more than once a week, once or more than once a month).

In a particularly preferred embodiment, the pharmaceutical composition comprises
(I) at least one first component, preferably at least one mRNA encoding a therapeutic peptide or protein, e.g. an antibody, an enzyme, an antigen, wherein, preferably, said mRNA does, optionally, not comprise modified nucleotides, wherein said mRNA does comprise a Cap1 structure (preferably obtainable by co-transcriptional capping); and
(II) at least one second component, preferably at least one single stranded RNA oligonucleotide comprising at least one 2′-O-methylated RNA nucleotide, preferably comprising a nucleic acid sequence according to formula I; and

wherein, preferably, said first component and said second component of the composition are co-formulated in a lipid nanoparticle as defined herein or co-formulated in a polyethylene glycol/peptide polymer as defined herein.

Kit or Kit of Parts

In a third aspect, the present invention provides a kit or kit of parts, preferably comprising the individual components of the combination (e.g. as defined in the context of the first aspect) and/or comprising the pharmaceutical composition of (e.g. as defined in the context of the second aspect).

Notably, embodiments relating to the first and the second aspect of the invention are likewise applicable to embodiments of the third aspect of the invention, and embodiments relating to the third aspect of the invention are likewise applicable to embodiments of the first and second aspect of the invention.

In preferred embodiments of the third aspect, the kit or kit of parts comprises at least one first and at least one second component as defined in the context of the first aspect, and/or at least one composition as defined in the context of the second aspect, optionally comprising a liquid vehicle for solubilizing, and, optionally, technical instructions providing information on administration and/or dosage of the components.

In preferred embodiments, the kit or the kit of parts comprises:
(a) at least one first component as defined herein, preferably an mRNA encoding a therapeutic peptide or protein, e.g. an antibody, an enzyme, an antigen, preferably wherein said mRNA does not comprise modified nucleotides, preferably wherein said mRNA does comprise a Cap1 structure, preferably wherein said first component is formulated in a lipid nanoparticle or in a polyethylene glycol/peptide polymer.
(b) at least one second component as defined herein, preferably a single stranded RNA oligonucleotide comprising at least one 2′-O-methylated RNA nucleotide, preferably comprising a nucleic acid sequence according to formula I, preferably wherein said second component is formulated in a lipid nanoparticle or in a polyethylene glycol/peptide polymer.
(c) optionally, a liquid vehicle for solubilising (a) and/or (b), and optionally technical instructions providing information on administration and dosage of the components.

In preferred embodiments, the kit or the kit of parts comprises:
(a) at least one composition as defined in the context of the second aspect;
(b) optionally, a liquid vehicle for solubilising, and optionally technical instructions providing information on administration and dosage of the components.

Embodiments and features disclosed in the context of the first and the second component, or the composition of the second aspect, are likewise applicable for the RNA and/or the composition of the kit or the kit of parts.

The kit or kit of parts may further comprise additional components as described in the context of the first or second component, or the composition, in particular, pharmaceutically acceptable carriers, excipients, buffers and the like.

The technical instructions of said kit or kit of parts may comprise information about administration and dosage and patient groups. Such kits, preferably kits of parts, may be applied e.g. for any of the applications or medical uses mentioned herein.

Preferably, the individual components of the kit or kit of parts may be provided in lyophilised form. The kit may further contain as a part a vehicle (e.g. pharmaceutically acceptable buffer solution) for solubilising the therapeutic RNA of the first component, and/or the antagonist, preferably the nucleic acid of the second component, and/or the composition of the second aspect.

In preferred embodiments, the kit or kit of parts comprises Ringer- or Ringer lactate solution.

In preferred embodiments, the kit or kit of parts comprise an injection needle, a microneedle, an injection device, a catheter, an implant delivery device, or a micro cannula.

Any of the above kits may be used in applications or medical uses as defined in the context of the invention.

Medical Use:

A further aspect relates to the first medical use of the provided combination, composition, or kit.

Embodiments described below (in the context of the “method of treatment”) are also applicable to first medical use and the further medical uses as described herein.

Accordingly, the invention provides a combination as defined in the context of the first aspect for use as a medicament, the composition as defined in the second aspect for use as a medicament, and the kit or kit of parts as defined in the third aspect for use as a medicament.

In particular, said combination, composition, or the kit or kit of parts may be used for human medical purposes and also for veterinary medical purposes, preferably for human medical purposes.

In particular, said combination, composition, or the kit or kit of parts is for use as a medicament for human medical purposes, wherein said combination, composition, or the kit or kit of parts may be particularly suitable for young infants, newborns, immunocompromised recipients, as well as pregnant and breast-feeding women and elderly people.

A further aspect relates to further medical uses of the provided combination, composition, or kit.

Accordingly, the invention provides a combination as defined in the context of the first aspect for use as a medicament, the composition as defined in the second aspect for use as a medicament, and the kit or kit of parts as defined in the third aspect for use as a chronic medical treatment.

The term “chronic medical treatment” relates to treatments that require the administration of the combination, the composition, or the kit or kit of parts more than once, for example once or more than once a day, once or more than once a week, once or more than once a month.

The invention further provides a combination as defined in the context of the first aspect, the composition as defined in the second aspect, and the kit or kit of parts as defined in the third aspect for use in the treatment or prophylaxis of an infection, or of a disorder related to such an infection. Preferably, the infection is selected from a virus infection, a bacterial infection, a protozoan infection. Accordingly, in said embodiments, the therapeutic RNA encodes at least one antigen.

The invention further provides a combination as defined in the context of the first aspect, the composition as defined in the second aspect, and the kit or kit of parts as defined in the third aspect for use in the treatment or prophylaxis of a tumour disease, or of a disorder related to such tumour disease. Accordingly, in said embodiments, the therapeutic RNA may encode at least one tumour or cancer antigen and/or at least one therapeutic antibody (e.g. checkpoint inhibitor).

The invention further provides a combination as defined in the context of the first aspect, the composition as defined in the second aspect, and the kit or kit of parts as defined in the third aspect for use in the treatment or prophylaxis of a genetic disorder or condition.

The invention further provides a combination as defined in the context of the first aspect, the composition as defined in the second aspect, and the kit or kit of parts as defined in the third aspect for use in the treatment or prophylaxis of a protein or enzyme deficiency or protein replacement. Accordingly, in said embodiments, the therapeutic RNA encodes at least one protein or enzyme. “Protein or enzyme deficiency” in that context has to be understood as a disease or deficiency where at least one protein is deficient, e.g. AlAT deficiency.

Methods of Treatment and Delivery:

A further aspect of the present invention relates to a method of treating or preventing a disease, disorder, or condition.

Embodiments described above (in the context of the first medical use and the further medical uses) are also applicable to methods of treatment as described herein.

In preferred embodiments of the third aspect, the method of treating or preventing a disorder, disease, or condition comprises a step of applying or administering to a subject the combination of the first aspect, the composition of the second aspect, or the kit or kit of parts of the second aspect.

The combination is preferably administered as a “co-administration” The term “co-administration” generally refers to the administration of at least two different substances sufficiently close in time. Co-administration refers to simultaneous administration, as well as temporally spaced order of up to several days apart, of at least two different substances in any order, either in a single dose or separate doses.

In preferred embodiments, applying or administering of the first component and the second component is performed essentially simultaneous (as defined herein).

In some embodiments the antagonist and the therapeutic RNA as defined herein are administered simultaneously as part of the same composition. In some embodiments the antagonist and the therapeutic RNA as defined herein are administered simultaneously as different compositions. In some embodiments, the antagonist and therapeutic RNA are administered by the same route of administration. In some embodiments, the antagonist and the therapeutic RNA are administered by different routes of administration.

In preferred embodiments, applying or administering of the first component and the second component is performed sequential (as defined herein). In some embodiments, the antagonist is administered prior to the therapeutic RNA. In some embodiments, the therapeutic RNA is administered prior to the antagonist. In some embodiments, the antagonist and therapeutic RNA are administered by the same route of administration. In some embodiments, the antagonist and the therapeutic RNA are administered by different routes of administration.

In preferred embodiments, applying or administering of the combination of the first aspect, the composition of the second aspect, or the kit or kit of parts of the third aspect is performed more than once, for example once or more than once a day, once or more than once a week, once or more than once a month (as defined herein).

Administration may be orally, parenterally, by inhalation spray, topically, rectally, nasally, buccally, vaginally or via an implanted reservoir. The term parenteral, as used herein, includes subcutaneous, intravenous, intramuscular, intra-articular, intra-synovial, intrasternal, intrathecal, intrahepatic, intralesional, intracranial, transdermal, intradermal, intrapulmonal, intraperitoneal, intracardial, intraarterial, intraocular, intravitreal, subretinal, intratuomoral.

In preferred embodiments, the step of applying or administering is subcutaneous, intravenous, intramuscular, intra-articular, intra-synovial, intrasternal, intrathecal, intrahepatic, intralesional, intracranial, transdermal, intradermal, intrapulmonal, intraperitoneal, intracardial, intraarterial, intraocular, intravitreal, subretinal, or intratuomoral.

In preferred embodiments, the subject in need is a mammalian subject, e.g. cattle, pigs, horses, sheep, cats, dogs; and/or birds, including commercially relevant birds such as poultry, chickens, ducks, geese, and/or turkeys. In particularly preferred embodiments, the subject in need is a human subject.

Methods of Reducing or Suppressing (Innate) Immune Stimulation of a Therapeutic RNA:

A further aspect of the present invention relates to a method of reducing or suppressing (innate) immune stimulation induced by a therapeutic RNA. By reducing or suppressing immune stimulation induced by a therapeutic RNA, the efficiency (e.g. translation of the therapeutic RNA, activity of the therapeutic RNA) upon administration may be increased. Accordingly, the herein described “method of reducing or suppressing (innate) immune stimulation of a therapeutic RNA” is also to be understood as a “method of increasing the efficiency of a therapeutic RNA”.

In preferred embodiments, the method comprises the steps of administering to a subject the at least one therapeutic RNA (as defined herein) and, additionally, the at least one antagonist of at least one RNA sensing pattern recognition receptor.

The at least one antagonist of at least one RNA sensing pattern recognition receptor may be provided as separate entity (e.g. as described in the context of the combination of the first aspect) or provided as a single composition comprising the at least one therapeutic RNA and, additionally, the at least one antagonist of the at least one RNA sensing pattern recognition receptor.

Advantageously, administration of said antagonist reduces the innate immune responses that may be induced by the therapeutic RNA (without e.g. affecting the translation of an e.g. therapeutic coding RNA). Suitably, reducing the stimulation of innate immune responses may be advantageous for various medical applications of the therapeutic RNA. In particular, the method may e.g. enable the chronic administration of a therapeutic RNA or may e.g. enhance or improve the therapeutic effect of a therapeutic RNA encoding an antigen (e.g. viral antigen, tumour antigen). Accordingly, reducing the innate immune responses of the therapeutic RNA of the invention leads to an increased efficiency of a therapeutic RNA (e.g. upon administration to a cell or a subject).

Moreover, in that context, the method allows the reduction of reactogenicity of a coding therapeutic RNA (comprising a cds encoding e.g. an antigen). The term reactogenicity refers to the property of e.g. a vaccine of being able to produce adverse reactions, especially excessive immunological responses and associated signs and symptoms-fever, sore arm at injection site, etc. Other manifestations of reactogenicity typically comprise bruising, redness, induration, and swelling.

Accordingly, the method of method of reducing or suppressing (innate) immune stimulation of a therapeutic RNA has also be understood as method of reducing or suppressing the reactogenicity of a coding therapeutic RNA, wherein said coding RNA comprises a cds encoding an antigen.

Methods of Increasing and/or Prolonging Expression of a (Coding) Therapeutic RNA:

A further aspect of the present invention relates to a method of increasing and/or prolonging expression of a coding therapeutic RNA. By increasing and/or prolonging expression of a coding therapeutic RNA, the efficiency (e.g. translation of the therapeutic RNA, activity of the therapeutic RNA) upon administration may substantially be increased. Accordingly, the herein described “method of increasing and/or prolonging expression of a (coding) therapeutic RNA” is also to be understood as a “method of increasing the efficiency of a (coding) therapeutic RNA”.

In preferred embodiments, the method comprises the steps of administering to a subject at least one coding therapeutic RNA (as defined herein) and, additionally, the at least one antagonist of at least one RNA sensing pattern recognition receptor.

The at least one antagonist of at least one RNA sensing pattern recognition receptor may be provided as separate entity (e.g. as described in the context of the combination of the first aspect) or provided as a single composition comprising the at least one therapeutic RNA and, additionally, the at least one antagonist of the at least one RNA sensing pattern recognition receptor.

Advantageously, administration of said antagonist reduces the suppression of protein translation that may be induced by the therapeutic RNA. Suitably, increasing and/or prolonging may be advantageous for various medical applications of the therapeutic RNA. In particular, the method may e.g. enable the chronic administration of a therapeutic RNA or may e.g. enhance or improve the therapeutic effect of a therapeutic RNA encoding an antigen (e.g. viral antigen, tumour antigen). Accordingly, increasing and/or prolonging of the therapeutic RNA of the invention leads to an increased efficiency of a therapeutic RNA (e.g. upon administration to a cell or a subject).

### BRIEF DESCRIPTION OF LISTS AND TABLES

Table A: Preferred small molecule antagonists of the invention
Table B: Preferred oligonucleotide antagonists of the invention
Table 1: Human codon usage with respective codon frequencies indicated for each amino acid
Table 2: Combination of RNA constructs for DOTAP formulation with 2′-O-methylated oligonucleotide
Table 3: Constructs and dose of PpLuc mRNA and 2′-O-methylated oligonucleotide for analysis of expression and immunostimulation in vivo
Table 4: Injection schedule for analysis of expression and immunostimulation in vivo
Table 5: Time points and experimental setup for analysis of immunostimulation in vivo

### BRIEF DESCRIPTION OF THE DRAWINGS

FIG. 1A shows the immunosuppressive effect of the addition of the 2′-O-methylated oligonucleotide (“Gm18”) to an immunostimulatory non-coding RNA (“RNAdjuvant”) in PBMCs in vitro. The DOTAP co-transfection of uncapped immunostimulatory non-coding RNA and the 2′-O-methylated oligonucleotide shows a reduction in cytokine response compared to transfection of immunostimulatory non-coding RNA only measured by CBA array in PBMCs supernatant. Vehicle=DOTAP only; Further details are provided in Example 2.

FIG. 1B shows the immunosuppressive effect of the addition of the 2′-O-methylated oligonucleotide (“Gm18”) to PpLuc mRNA in PBMCs in vitro. The DOTAP co-transfection of capped coding PpLuc mRNA and the oligonucleotide shows a reduction in cytokine response compared to transfection of PpLuc mRNA only, measured by CBA array in PBMCs supernatant. Vehicle=DOTAP only; Further details are provided in Example 2.

FIG. 2 shows the expression of PpLuc from mRNA with and without admixture of 2′-O-methylated RNA (“Gm18”) oligonucleotide at 6 hours and 24 hours post intravenous injection of LNP in 129Sv mice. To quantify PpLuc expression, bioluminescence was recorded for 3 minutes starting 5 minutes after i.v. injection of 3 mg of luciferin. The addition of the 2′-O-methylated RNA oligonucleotide increases the expression of PpLuc at 24 hours post injection compared to PpLuc mRNA without 2′-O-methylated RNA oligonucleotide at either dose (10 μg or 30 μg of mRNA). Further details are provided in Example 2.

FIG. 3 shows the expression of PpLuc in liver lysates after single intravenous injection of PpLuc mRNA with and without admixture of 2′-O-methylated oligonucleotide (“Gm18”) formulated in LNP in mice. Livers were collected 24 hours post injection of 10 μg or 30 μg of mRNA. The addition of the 2′-O-methylated RNA oligonucleotide increases the expression of PpLuc at 24 hours post injection compared to PpLuc mRNA without 2′-O-methylated RNA oligonucleotide at either dose. Further details are provided in Example 3.

FIG. 4A shows the immunosuppressive effect of the addition of the 2′-O-methylated oligonucleotide (“Gm18”) to PpLuc mRNA 6 hours post injection formulated in LNP in mice. A CBA array was performed with sera obtained 6 hours post intravenous injection to compare the cytokine levels (RANTES, IL6, MCP1, MCP-1β, TNFα and IFNγ) induced by co-formulated mRNA+2′-O-methylated oligonucleotide or by formulated mRNA only. All cytokine levels are strongly reduced by admixture of the 2′-O-methylated oligonucleotide in a dose-dependent manner. Further details are provided in Example 3.

FIG. 4B shows the immunosuppressive effect of the addition of the 2′-O-methylated oligonucleotide (“Gm18”) to PpLuc mRNA 24 hours post injection formulated in LNP in mice. An ELISA was performed with sera obtained 24 hours post intravenous injection to compare the level of INFa induced by co-formulated mRNA+2′-O-methylated oligonucleotide or by formulated mRNA only. INFα levels are strongly reduced by admixture of the 2′-O-methylated oligonucleotide in a dose-dependent manner. Further details are provided in Example 3.

FIG. 5A shows the immunosuppressive effect of the addition of the 2′-O-methylated oligonucleotide variants, RNA oligonucleotides, DNA oligonucleotides and small molecules to PpLuc mRNA in PBMCs in vitro. The DOTAP co-transfection of capped coding PpLuc mRNA and the oligonucleotides and small molecules shows a reduction in cytokine response (IFN-α) compared to transfection of PpLuc mRNA only, measured by CBA array in PBMCs supernatant. Vehicle=DOTAP only; Further details are provided in Example 4

FIG. 5B shows the expression of PpLuc from mRNA with and without admixture of the 2′-O-methylated oligonucleotide (“Gm18”), 2′-O-methylated oligonucleotide variants, RNA oligonucleotides, DNA oligonucleotides and small molecules to PpLuc mRNA in PBMCs in vitro. To quantify PpLuc expression, bioluminescence was recorded for 3 minutes starting 5 minutes after i.v. injection of 3 mg of luciferin. The addition of the 2′-O-methylated oligonucleotide (“Gm18”), 2′-O-methylated oligonucleotide variants, RNA oligonucleotides, DNA oligonucleotides and small molecules increases the expression of PpLuc at 24 hours post transfection compared to PpLuc mRNA without admixture

### EXAMPLES

The following examples are given to enable those skilled in the art to more clearly understand and to practice the present invention. The present invention, is not limited in scope by the exemplified embodiments, which are intended as illustrations of single aspects of the invention only, and methods which are functionally equivalent are within the scope of the invention. Indeed, various modifications of the invention in addition to those described herein will become readily apparent to those skilled in the art from the foregoing description, accompanying figures and the examples below.

### Example 1: Generation RNA Constructs

1.1. Preparation of DNA Templates

A DNA sequence encoding luciferase was prepared and used for subsequent RNA in vitro transcription. Said DNA sequence was prepared by modifying the wild type cds sequences by introducing a GC optimized cds. Sequences were introduced into a plasmid vector to comprising UTR sequences, a stretch of adenosines, a histone-stem-loop structure, and, optionally, a stretch of 30 cytosines. Obtained plasmid DNA was transformed and propagated in bacteria using common protocols and plasmid DNA was extracted, purified, and used for subsequent RNA in vitro transcription as outlined below.

A DNA sequence encoding immunostimulatory non-coding RNA was prepared and used for subsequent RNA in vitro transcription. Obtained plasmid DNA was transformed and propagated in bacteria using common protocols and plasmid DNA was extracted, purified, and used for subsequent RNA in vitro transcription.

1.2. RNA In Vitro Transcription from Plasmid DNA Templates:

1.2.1. Preparation of mRNA Encoding PPluc:

DNA plasmids prepared according to section 1.1 were enzymatically linearized using a restriction enzyme and used for DNA dependent RNA in vitro transcription using T7 RNA polymerase in the presence of a nucleotide mixture (ATP/GTP/CTP/UTP) and cap analogue (e.g., m7GpppG or m7G(5′)ppp(5′)(2′OMeA)pG or m7G(5′)ppp(5′)(2′OMeG)pG)) under suitable buffer conditions. The obtained RNA was purified using RP-HPLC (PureMessenger®; WO2008/077592) and used for in vitro and in vivo experiments.

1.2.2. Preparation of Immunostimulatory Non-Coding RNA:

DNA plasmids prepared according to section 1.1 were enzymatically linearized using a restriction enzyme and used for DNA dependent RNA in vitro transcription using T7 RNA polymerase in the presence of a nucleotide mixture (ATP/GTP/CTP/UTP) under suitable buffer conditions. The obtained non-coding RNA was purified using RP-HPLC (PureMessenger®; WO2008/077592) and used for in vitro and in vivo experiments.

### Example 2: Immunostimulation of Human Peripheral Blood Mononuclear Cells (PBMCs) by Co-Transfection of 2′-O-methylated Oligonucleotide and RNA

For the example described below a 2′-O-methylated oligonucleotide (9-mer) was synthesized by Biomers (biomers.net GmbH, Germany): 5′-GAG CGmG CCA-3′ (SEQ ID NO 85), also herein referred to as “Gm18”.

2.1 Preparation of Human PBMCs

Human peripheral blood mononuclear cells (PBMCs) were isolated from heparinized blood of healthy volunteers by standard Ficoll-Hypaque density gradient centrifugation (Ficoll 1.078 g/ml). PBMCs were re-suspended in RPMI 1640 supplemented with 10% heat-inactivated FCS. After counting, cells are re-suspended at 50 million cells per ml in fetal calf serum, 10% DMSO, and frozen. Before usage, the cells are thawed.

2.2 PBMC Stimulation

For transfection experiments, 2×10⁵ human PBMCs per well were seeded into each well of a 96-well plate in X-Vivo 15 medium (Lonza). For preparation of DOTAP complexes containing both immunostimulatory non-coding RNA and a 2′-O-methylated oligonucleotide (SEQ ID NO: 85), the oligonucleotide was first added to immunostimulatory non-coding RNA at a weight percentage of 25%. For preparation of DOTAP complexes containing both PpLuc mRNA and a 2′-O-methylated oligonucleotide, the oligonucleotide was first added to PpLuc mRNA at a weight percentage of 25%. The molar ratio of PpLuc mRNA to oligonucleotide was thus 1:45 (MW (Oligonucleotide)=2907 g/mol, MW (PpLuc mRNA)=652377 g/mol). DOTAP complexes containing either immunostimulatory non-coding RNA or PpLuc mRNA without or with oligonucleotide were formed at a ratio of 3 μl of DOTAP per 1 μg of RNAdjuvant or 1 μg of mRNA. PBMC were incubated overnight with 1 μg/ml of immunostimulatory non-coding RNA or mRNA without or with 0.25 μg/ml of oligonucleotide in a total volume of 200 μl in a humidified 5% C02 atmosphere at 37° C. To quantify background stimulation, PBMC were incubated either with DOTAP alone (“vehicle”) or medium only. 24 hours after transfection, supernatants were collected.

TABLE 2
Combination of RNA constructs for DOTAP formulation
with 2‘-O-methylated oligonucleotide

| | RNA Design | | | |
|---|---|---|---|---|
| | | UTR | Poly(A) | |
| | | design | sequence, | Gm18 |
| | 5’-cap | 5’-UTR/ | located at | oligo- |
| RNA ID | structure | 3’-UTR | 3’ terminus | nucleotide |
| immunostimulatory | / | / | / | 5’-GAG |
| non-coding RNA | | | | CGmG |
| (SEQ ID NO: 84) | | | | CCA-3’ |
| immunostimulatory | / | / | / | / |
| non-coding RNA | | | | |
| (SEQ ID NO: 84) | | | | |
| PpLuc mRNA | mCap | RPL32/ | A64N5C30. | 5’-GAG |
| (SEQ ID NO: 82) | | ALB7 | Hs_HSL | CGmG |
| | | | | CCA-3’ |
| PpLuc mRNA | mCap | RPL32/ | A64N5C30. | / |
| (SEQ ID NO: 82) | | ALB7 | Hs_HSL | |

2.3 Cytometric Bead Array (CBA)

In supernatants collected from PBMC stimulated without or with 2-O-methylated oligonucleotide, the concentrations of IFN-α, IFN-γ, TNF, were measured by Cytometric Bead Array (CBA) according to the manufacturer's instructions (BD Biosciences) using the following kits: Human Soluble Protein Master Buffer Kit (catalog no. 558264), Assay Diluent (catalog no. 560104), Human IFN-α Flex Set (catalog no. 560379), Human IFN-γ Flex Set (catalog no. 558269), Human TNF Flex Set (catalog no. 560112); all kits from BD Biosciences. The data was analyzed using the FCAP Array v3.0 software (BD Biosciences).

2.4 Results: Immunosuppressive Effect of the Addition of the 2′-O-Methylated Oligonucleotide

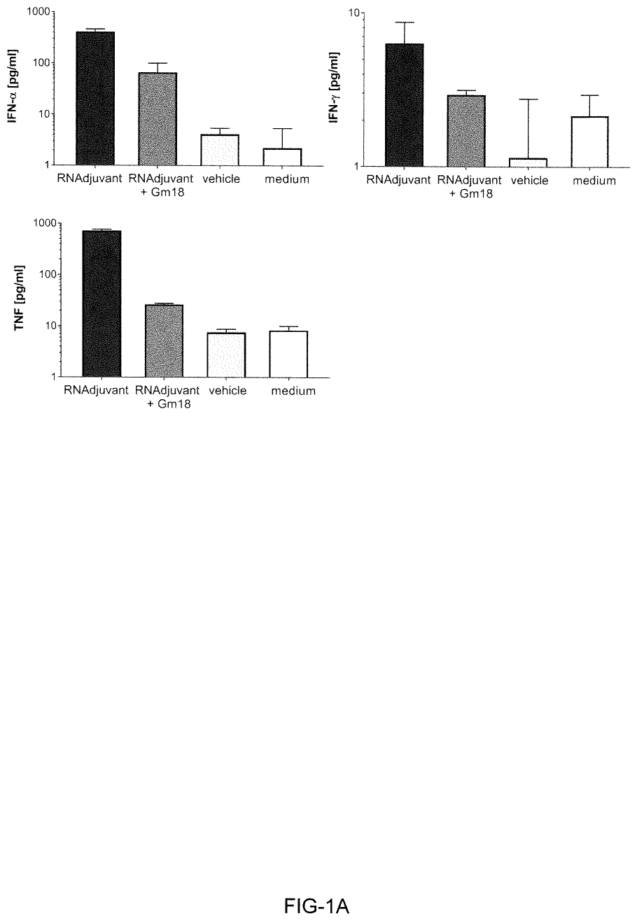

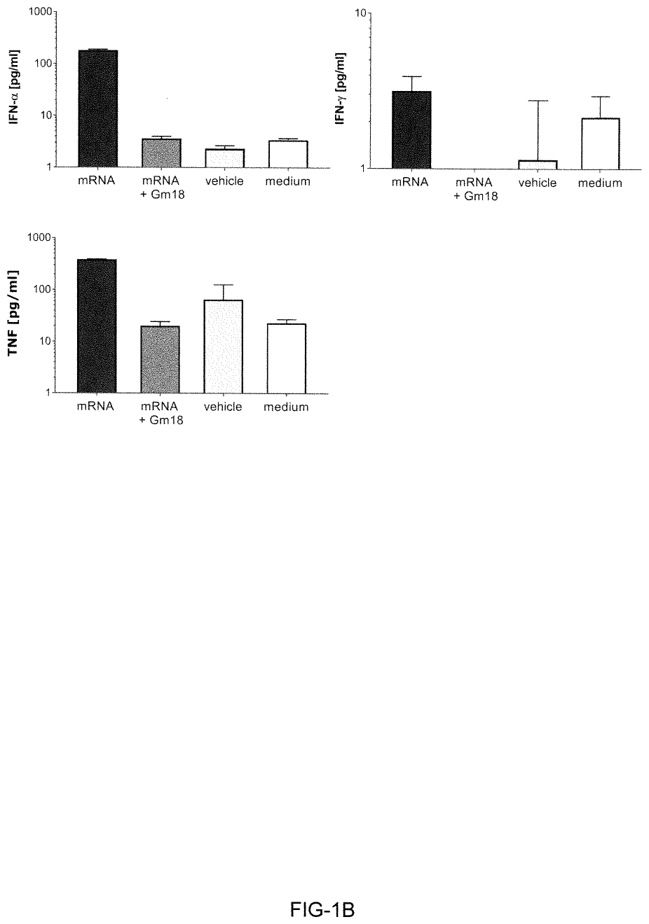

DOTAP co-transfection of the 2′-O-methylated oligonucleotide (“Gm18”) together with an immunostimulatory non-coding RNA (“RNAdjuvant”) in human PBMCs demonstrates an immunosuppressive effect of the 2′-O-methylated oligonucleotide evidenced by reduced secretion of cytokines INF-a, INF-y, and TNF compared to transfection of immunostimulatory non-coding RNA only (FIG. 1A).

DOTAP co-transfection of the 2′-O-methylated oligonucleotide (“Gm18”) together with capped coding PpLuc mRNA in human PBMCs demonstrates an immunosuppressive effect of the 2-O-methylated oligonucleotide by reduced secretion of the cytokines INF-a, INF-y, and TNF compared to transfection of PpLuc mRNA only (FIG. 1B).

The results show that the 2′-O-methylated oligonucleotide tested herein is able to reduce immunostimulation of RNA, suggesting that a combination or composition comprising oligonucleotide and therapeutic RNA may show reduced immunostimulatory properties.

### Example 3: Immunostimulation of PpLuc mRNA in Combination with 2′O-Methylated Oligonucleotide with LNP In Vivo

For the example described below a 9-mer 2-O-methylated oligonucleotide (9-mer) was synthesized by Biomers (biomers.net GmbH, Germany): 5′-GAG CGmG CCA-3′ (SEQ ID NO 85).

3.1 Generation of PpLuc mRNA Constructs

mRNA constructs encoding PpLuc were generated according to Example 1.

3.2 LNP Formulation

For preparation of Lipid nanoparticles (LNP) containing both PpLuc mRNA and 2-O-methylated oligonucleotide, first the 2′-O-methylated oligonucleotide was added to PpLuc mRNA at a weight percentage of either 20% or 6.7% (see Table 3). LNP containing PpLuc mRNA either with or without admixture of 2-O-methylated oligonucleotide were prepared using cationic lipid, cholesterol, PEG-lipid and a neutral lipid. The mRNA was diluted to 1 g/L in citrate buffer, pH 4. The ethanolic lipid solution was mixed with the aqueous RNA solution at a ratio of 1:3 (vol/vol) using a Nanoassemblr (PrecisionNanoSystems). The ethanol was then removed and the buffer replaced by 10 mM HEPES, pH 7.4 comprising 9% Sucrose by dialysis. Finally, the LNP-formulated RNA was adjusted to 0.2 g/L.

TABLE 3
Constructs and doses of PpLuc mRNA and 2′-O-methylated
oligonucleotide for analysis of expression
and immunostimulation in vivo

| | | | | | | LNP |
|---|---|---|---|---|---|---|
| | Cap | | | mRNA | % mass | formulation |
| Group | structure | 5‘UTR | 3′UTR | dose | of Oligo | @0.2 g/L |
| 1 | Cap1 | HSD17B4 | PSMB3 | 30 μg | 0 | A |
| 2 | Cap1 | HSD17B4 | PSMB3 | 10 μg | 0 | A |
| 3 | Cap1 | HSD17B4 | PSMB3 | 30 μg | 20% | B |
| 4 | Cap1 | HSD17B4 | PSMB3 | 10 μg | 20% | B |
| 5 | Cap1 | HSD17B4 | PSMB3 | 30 μg | 6.7% | C |
| 6 | Cap1 | HSD17B4 | PSMB3 | 10 μg | 6.7% | C |

TABLE 4
Injection schedule for analysis of expression
and immunostimulation in vivo

| | | Concentration | |
|---|---|---|---|
| | Formu- | for | |
| RNA | lation | injection | schedule |
| PpLuc mRNA | A | 0.2 g/l | 4 mice/group; |
| (SEQ ID NO: 83) | | | 10 μg and |
| | | | 30 μg dose |
| | | | (i.v.) |
| PpLuc mRNA | B | 0.2 g/l | 4 mice/group; |
| (SEQ ID NO: 83) | | | 10 μg and |
| and | | | 30 μg dose |
| 2‘-O-methylated | | | (i.v.) |
| oligonucleotide | | | |
| (SEQ ID NO 85) | | | |
| % mass of oligo: 20% | | | |
| PpLuc mRNA | C | 0.2 g/l | 4 mice/group; |
| (SEQ ID NO: 83) | | | 10 μg and |
| and | | | 30 μg dose |
| 2‘-O-methylated | | | (i.v.) |
| oligonucleotide | | | |
| (SEQ ID NO 85) | | | |
| % mass of oligo: 6.7% | | | |

3.3 Intravenous Injection of PpLuc mRNA, 2′-O-Methylated Oligonucleotide and LNP in Mice

For in vivo experiments, 8 weeks old female mice (around 25 g, strain 129SV) were injected with the various LNP formulations (see tables 4 and 5). 4 animals were used per group. 10 μg or 30 μg of mRNA formulated with or without 2′-O-methylated oligonucleotide were intravenously injected at a concentration of 0.2 g/l. Bioluminescence imaging was performed 6 hours and 24 hours post LNP injection. Blood was sampled 6 hours post LNP injection and terminally 24 hours post LNP injection. Immediately thereafter, mice were sacrificed, livers collected and placed in 1.5 ml PP tubes, frozen and stored until analysis (<−70° C.).

3.4 Expression Analysis from In Vivo Imaging

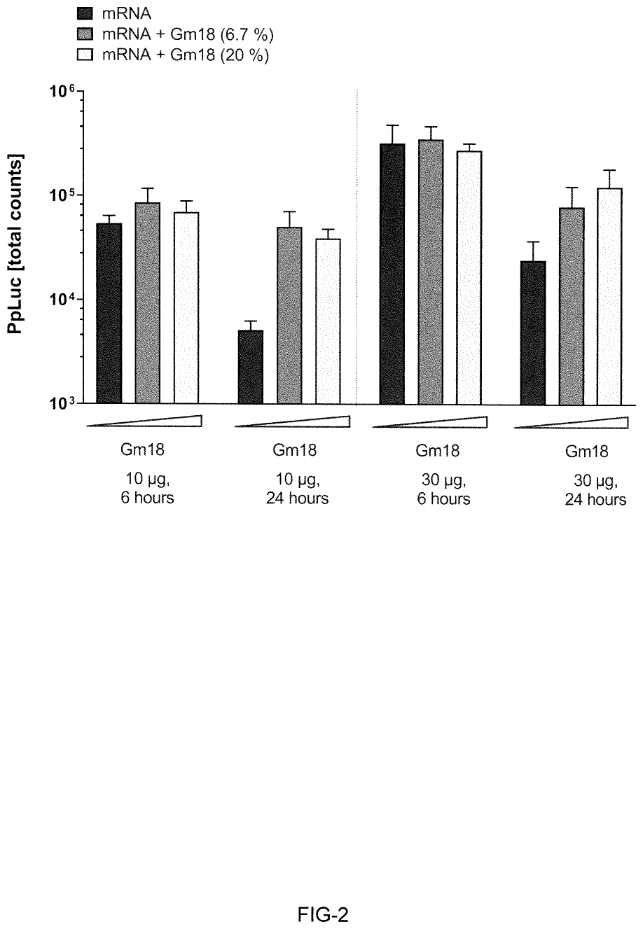

6 hours and 24 hours post single intravenous injection of LNP-formulated PpLuc mRNA without or with admixture of 2′-O-methylated oligonucleotide (“Gm18”), expression of PpLuc was visualized. PpLuc expression was quantified from bioluminescence images recorded for 3 minutes starting 5 minutes after i.v. injection of 3 mg of luciferin (see Table 5). The addition of the 2′-O-methylated oligonucleotide (“Gm18”) increases the expression of PpLuc at 24 hours post injection compared to PpLuc mRNA without Gm18 at either dose (10 μg or 30 μg of mRNA) (see FIG. 2).

TABLE 5
Time points and experimental setup
for analysis of immunostimulation in vivo

| | Formulation | Intra- | | | Organ |
|---|---|---|---|---|---|
| | (containing | venous | | | to be |
| | PpLuc | injec- | In-vivo | Serum | collected |
| Group | mRNA) | tion: | imaging | sampling | at 24 h |
| 1 | A | 30 μg | 6 and 24 hours | 6 and 24 hours | liver |
| 2 | A | 10 μg | 6 and 24 hours | 6 and 24 hours | liver |
| 3 | B | 30 μg | 6 and 24 hours | 6 and 24 hours | liver |
| 4 | B | 10 μg | 6 and 24 hours | 6 and 24 hours | liver |
| 5 | C | 30 μg | 6 and 24 hours | 6 and 24 hours | liver |
| 6 | C | 10 μg | 6 and 24 hours | 6 and 24 hours | liver |

3.5 Expression Analysis from Cell Lysates

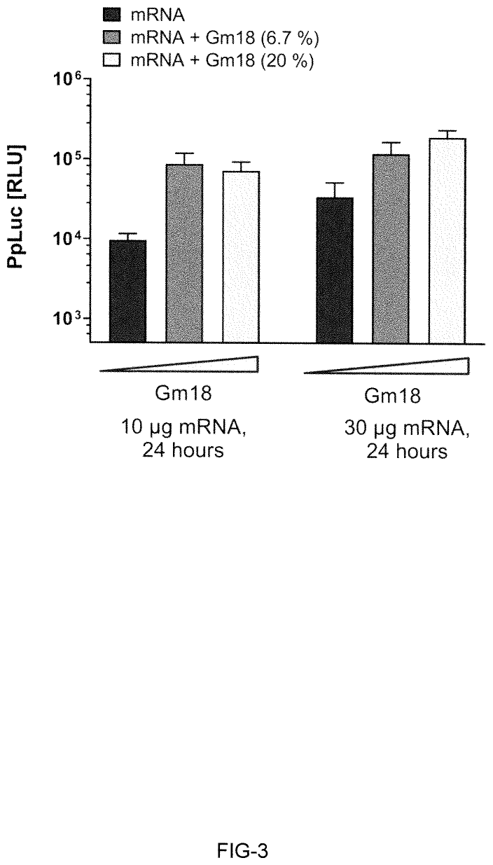

To prepare tissue lysates, first a steal bead was added to each liver. Frozen livers were mounted in a tissue lyser and shaken for three minutes. Then, 800 μl of Lysis Buffer was added (25 mM Tris-HCl pH 7.5, 2 mM EDTA, 10% (w/v) Glycerol, 1% (w/v) Triton X-100, 2 mM DTT, and 1 mM PMSF). Tissue lysis was continued for 6 more minutes. Samples were centrifuged at 13500 rpm at 4° C. for 10 min. 20 μl of each supernatant was added to white LIA assay plates. Plates were introduced into a plate reader (Berthold Technologies TriStar2 LB 942) and 50 μl per well of Beetle-Juice (PJK GmbH) containing luciferin as substrate for firefly luciferase was injected. Luciferase activity was quantified as relative light units (RLU). The addition of the 2′-O-methylated oligonucleotide increases the expression of PpLuc in lysates at 24 hours post injection compared to PpLuc mRNA without oligonucleotide at either dose (10 μg or 30 μg). (see FIG. 3).

3.6 Influence on Immunostimulation-CBA Assay and ELISA

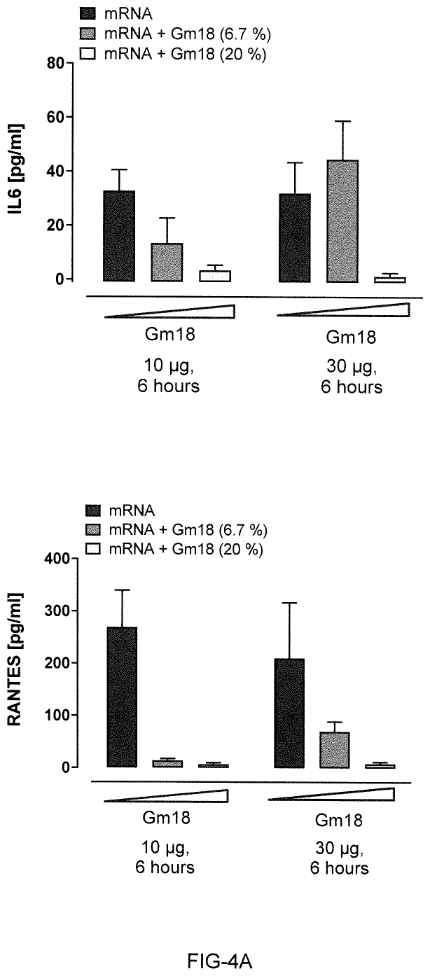

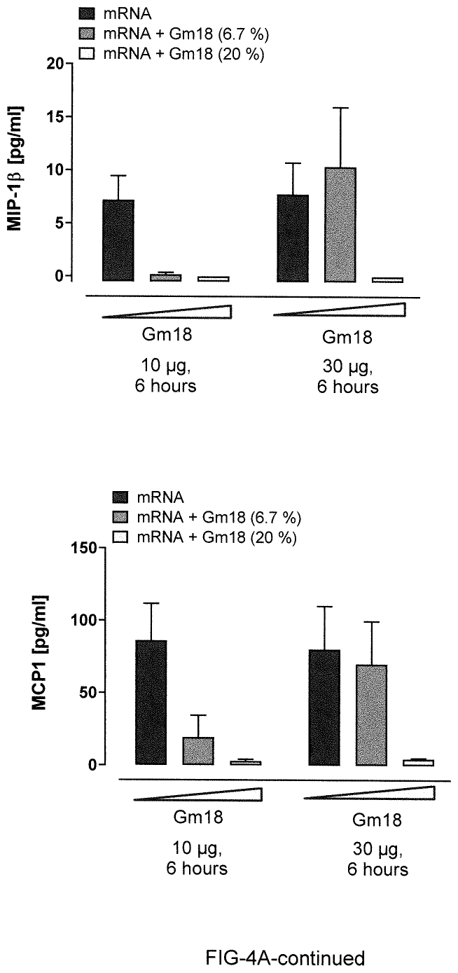

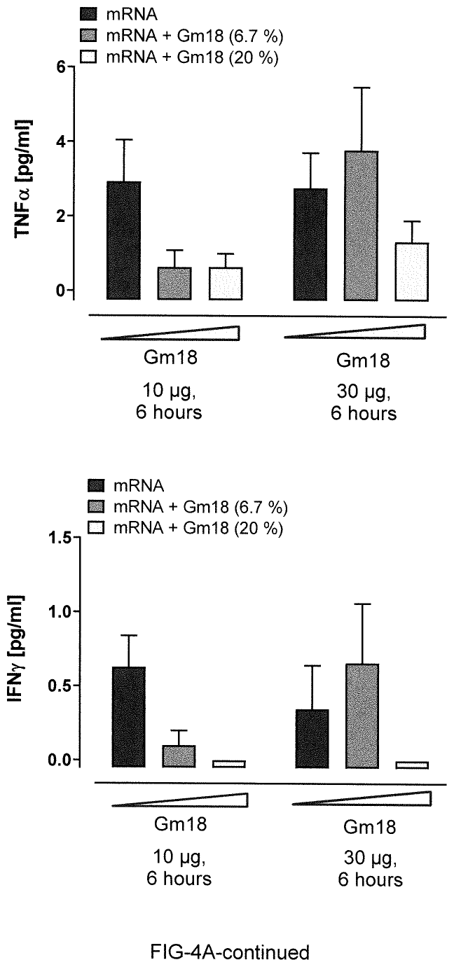

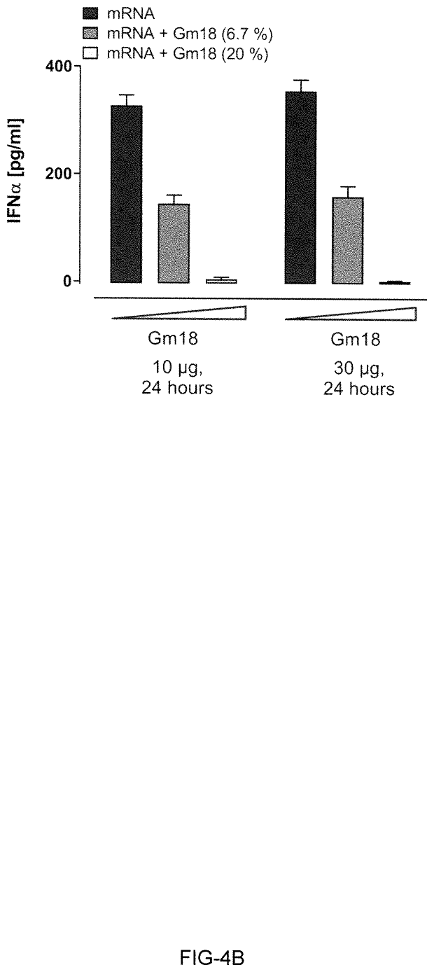

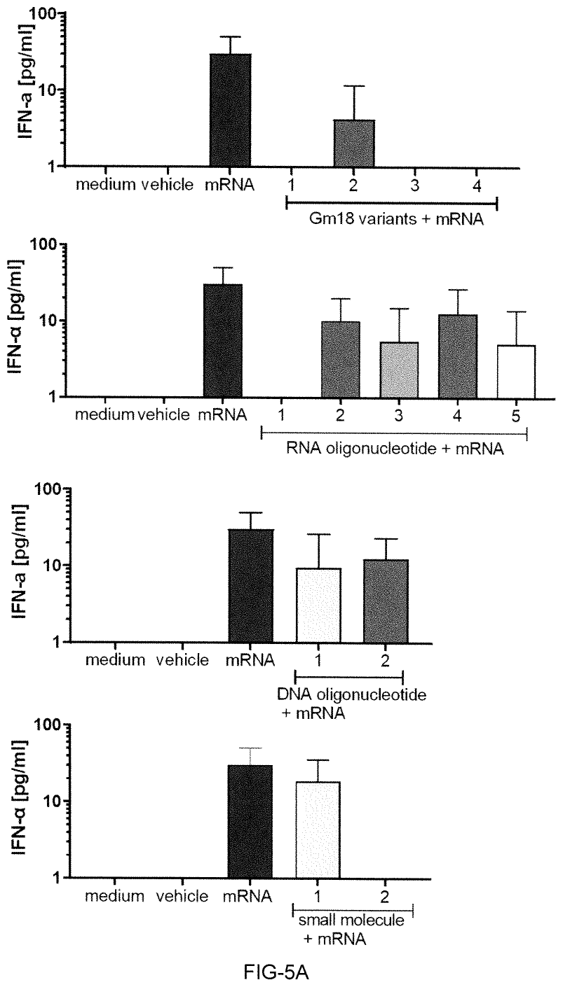

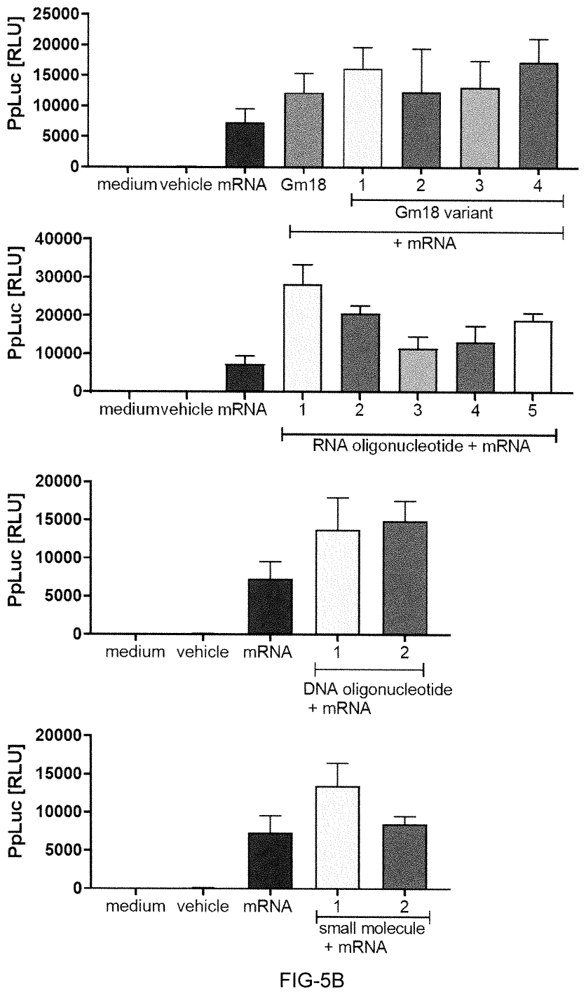

To analyse the influence of the 2′-O-methylated oligonucleotide on immunostimulation, the concentrations of IFNγ, TNFα, IL-6, MIP-1β, RANTES, and MCP1 were measured in sera from blood collected 6 hours post LNP injection by Cytometric Bead Array (CBA), performed as described in paragraph 2.3. Addition of the 2′-O-methylated RNA oligonucleotide to PpLuc mRNA strongly decreases the release of all inflammatory cytokines in a dose-dependent manner (see FIG. 4A). To further assess the influence of the 2′-O-methylated oligonucleotide on immunostimulation, the concentration of IFNα was measured in sera from blood collected 24 hours post LNP injection by ELISA. Addition of the 2′-O-methylated oligonucleotide to PpLuc mRNA strongly decreases the release of IFNα in a dose-dependent manner (see FIG. 4B).

### Summary of the Findings (Examples 1 to 3)

The results of the in vitro experiments described in Example 2, FIG. 1 show that the 2′-O-methylated oligonucleotide (“Gm18”) used herein antagonises the immunostimulation of a co-administered RNA, that is typically triggered by RNA sensing pattern recognition receptors. Accordingly, the oligonucleotide serves as an antagonist of RNA sensing pattern recognition receptors. The results show that a combination or composition comprising an oligonucleotide antagonist and a therapeutic RNA advantageously reduce the immunostimulatory properties of a therapeutic RNA. The results of the in vivo experiments described in Example 3, FIGS. 2 to 4 show that the 2-O-methylated oligonucleotide used herein antagonises the immunostimulation of an RNA also in vivo. Unexpectedly, the addition of the 2′-O-methylated oligonucleotide also increases/prolongs expression of the RNA encoded protein, suggesting that a combination or composition comprising oligonucleotide antagonist and therapeutic RNA shows, besides reduced immunostimulation, increased expression and/or activity in vivo-features that are of paramount importance for most RNA-based medicaments.

4. Immunostimulation of Human Peripheral Blood Mononuclear Cells (PBMCs) and Expression Efficiency by Co- Transfection of RNA and 2′-O-methylated Oligonucleotide Variants, RNA Oligonucleotides, DNA Oligonucleotides and Small Molecules

For the example described below the different oligonucleotides and small molecules were synthesized by Biomers (biomers.net GmbH, Germany), Invivogen (https://www.invivogen.com/, United States) or Miltenyi Biotec (miltenyibiotec.com/DE-en/, Germany) (Table 6).

4.1 Generation of PpLuc mRNA Constructs

mRNA constructs encoding PpLuc were generated according to Example 1.

TABLE 6
Synthesized 2′-O-methylated oligonucleotide variants, RNA oligonucleotides,
DNA oligonucleotides and small molecules

| | | SEQ | Synthesized |
|---|---|---|---|
| Name | Sequence | ID No | by |
| Gm 18 | GAGCGmGCCA | 85 | Biomers |
| Gm 18 variant 1 | G*A*G*C*Gm*G″C*C*A | 187 | Biomers |
| Gm 18 variant 2 | GAGCUmGCCA | 153 | Biomers |
| Gm 18 variant 3 | GCGmGCCAAA | 188 | Biomers |
| Gm 18 variant 4 | G*C*Gm*G*C*C*A*A*A | 189 | Biomers |
| RNA oligo 1 | Am*Um*A*Am*Um*U*U*U*Um*Um*G*G*U*Am*Um*U*U | 201 | Biomers |
| RNA oligo 2 | GAmUmUAmUGmUCCGGmUmUAmUGmUAUU | 107 | Biomers |
| RNA oligo 3 | UUGAUGmUGmUUUAGUCGCUAUU | 204 | Biomers |
| RNA oligo 4 | GGU GGG GUU CCC GAG CGmG CCA AAG GGA | 205 | Biomers |
| RNA oligo 5 | UmGmCmUmCmCmUmGmGmAmGmGmGmGmUmUmGmU | 203 | Biomers |
| DNA oligo 1 | T*C*C*T*G*G*C*G*G*G*G*A*A*G*T | 193 | Miltenyi |
| | | | Biotec |
| DNA oligo 2 | T*A*A*T*G*G*C*G*G*G*G*A*A*G*T | 194 | Miltenyi |
| | | | Biotec |
| small molecule 1 | C₁₇H₁₅NO₂ | | Invivogen |
| small molecule 2 | C₁₈H₂₆CIN₃ | | Invivogen |

* = Phosphorothioate backbone, Nm = methylated nucleotide (G, U, C or A)

4.2 Analysis of Expression and Immunostimulation of PMBCs Co-Transfected with 2′-O-Methylated Oligonucleotide Variants, RNA- and DNA Oliqonucleotides and Small Molecules

The preparation of human PBMCs was performed according to example 2.1. For transfection experiments 2×105 human PBMCs per well were seeded into each well of a 96-well plate in X-Vivo 15 medium (Lonza). For preparation of DOTAP (vehicle) complexes containing both the antagonist (either 2-O-methylated oligonucleotide variants, RNA oligonucleotides, DNA oligonucleotides or small molecules) as well as PpLuc mRNA (SEQ ID NO: 82, same RNA design as shown in table 2), the antagonist was first added to PpLuc mRNA at a weight percentage of 20% (1:5 mRNA: oligo/small molecule). The molar ratio of PpLuc mRNA to antagonist was thus 1:45 (MW (Oligonucleotide)=2907 g/mol, MW (PpLuc mRNA)=652377 g/mol). DOTAP complexes containing PpLuc mRNA and antagonist were formed at a ratio of 5 μl of DOTAP per 1 μg of mRNA and 100 ng were transfected. PBMC were incubated overnight with mRNA without or with 0.25 μg/ml of antagonist in a total volume of 200 μl in a humidified 5% C02 atmosphere at 37° C. To quantify background stimulation, PBMC were incubated either with DOTAP alone (“vehicle”) or RPMI (“medium”) only. 24 hours after transfection, supernatants were collected and cells were lysed and stored at −80° C. Cytrometric bead assay (CBA) was performed according to 2.3. Expression analysis was performed by measuring the luciferase activity, which is measured as relative light units (RLU) in a BioTek SynergyHT plate reader. PpLuc activity is measured at 5 seconds measuring time using 50 μl of lysate and 200 μl of luciferin buffer (75 μM luciferin, 25 mM Glycylglycin, pH 7.8 (NaOH), 15 mM MgSO4, 2 mM ATP).

4.3 Immunosuppressive Effect and Analysis of Expression Efficiency of PMBCs Co-Transfected with 2′-O-Methylated Oligonucleotide Variants. RNA- and DNA Oliaonucleotides and Small Molecules

DOTAP co-transfection of variants of 2′-O-methylated oligonucleotide, RNA oligonucleotides, DNA oligonucleotides as well as small molecules together with capped coding PpLuc mRNA in human PBMCs demonstrates an immunosuppressive effect evidenced by reduced secretion of cytokine IFN-α compared to transfection of PpLuc mRNA only, measured by CBA array in PBMCs supernatant (FIG. 4A). The addition of 2′-O-methylated oligonucleotide variants, RNA- and DNA oligonucleotides as well as small molecules increase the expression of PpLuc in PBMCs at 24 hours post transfection compared to PpLuc mRNA itself (FIG. 4B).

1. A combination comprising or consisting of
(i) at least one first component comprising at least one therapeutic RNA; and
(ii) at least one second component comprising at least one antagonist of at least one RNA sensing pattern recognition receptor.

2. Combination of claim 1, wherein the at least one RNA sensing pattern recognition receptor induces cytokines upon binding of an RNA agonist.

3. Combination of claim 1 or 2, wherein the at least one RNA sensing pattern recognition receptor inhibits translation upon binding of an RNA agonist.

4. Combination of any of the preceding claims, wherein the at least one antagonist of the second component reduces cytokine induction by the at least one RNA sensing pattern recognition receptor upon binding of an RNA agonist and/or reduces translation inhibition by the at least one RNA sensing pattern recognition receptor upon binding of an RNA agonist.

5. Combination of any of the preceding claims, wherein administration of the combination of the at least one therapeutic RNA of the first component and the at least one antagonist of at least one RNA sensing pattern recognition receptor of the second component leads to a reduced innate immune response compared to administration of the at least one therapeutic RNA of the first component without combination with the at least one antagonist of at least one RNA sensing pattern recognition receptor of the second component.

6. Combination of claim 5, wherein the induction of an innate immune response is determined by measuring the induction of cytokines.

7. Combination of claim 6, wherein the cytokines are selected from the group consisting of IFN-α, TNF-α, IP-10, IFN-γ, IL-6, IL-12, IL-8, Rantes, MIP-1 alpha, MIP-1 beta, McP1, or IFNbeta.

8. Combination of claim 6 or 7, wherein the induction of cytokines is measured by administration of the combination into cells, a tissue or an organism, preferably hPBMCs, Hela cells or HEK cells.

9. Combination of any of the preceding claims, wherein the at least one RNA sensing pattern recognition receptor is an endosomal receptor or a cytoplasmic receptor, preferably an endosomal receptor.

10. Combination of any of the preceding claims, wherein the at least one RNA sensing pattern recognition receptor is a receptor for single stranded RNA (ssRNA) and/or a receptor for double stranded RNA (dsRNA).

11. Combination of any of the preceding claims, wherein the at least one RNA sensing pattern recognition receptor is selected from a Toll-like receptor (TLR), a Retinoic acid-inducible gene-1-like receptor (RLR), a NOD-like receptor, PKR, OAS, SAMHD1, ADAR1, IFIT1 and/or IFIT5.

12. Combination of claim 11, wherein the at least one Toll-like receptor is selected from TLR3, TLR7, TLR8 and/or TLR9.

13. Combination of claim 11 or 12, wherein the at least one Toll-like receptor is selected from TLR8 and/or TLR9, most preferably from a TLR7 and/or TLR8.

14. Combination of claim 11, wherein the Retinoic acid-inducible gene-1-like receptor (RLR) is selected from RIG-1, MDA5, LGP2, cGAS, AIM2, NLRP3, NOD2, preferably RIG1 and/or MDA5.

15. Combination of any one of the preceding claims, wherein the at least one antagonist of the second component is selected from a nucleotide, a nucleotide analog, a nucleic acid, a peptide, a protein, a small molecule, a lipid, or a fragment, variant or derivative of any of these.

16. Combination of any one of the preceding claims, wherein the at least one antagonist of the second component is a nucleic acid.

17. Combination of any one of the preceding claims, wherein the at least one antagonist of the second component is a single stranded nucleic acid.

18. Combination of claim 16 or 17, wherein the nucleic acid of the second component comprises or consists of nucleotides selected from DNA nucleotides, RNA nucleotides, PNA nucleotides, and/or LNA nucleotides, or analogs or derivatives of any of these.

19. Combination of any one of claims 16 to 18, wherein the nucleic acid of the second component comprises at least one modified nucleotide and/or at least one nucleotide analogue or nucleotide derivative.

20. Combination of claim 19, wherein the at least one modified nucleotide and/or at least one nucleotide analogues is selected from a backbone modified nucleotide, a sugar modified nucleotide and/or a base modified nucleotide, or any combination thereof.

21. Combination of any one of claim 19 or 20, wherein the least one modified nucleotide and/or the at least one nucleotide analog is selected from 1-methyladenosine, 2-methyladenosine, N6-methyladenosine, 2′-O-methyladenosine, 2-methylthio-N6-methyladenosine, N6-isopentenyladenosine, 2-methylthio-N6-isopentenyladenosine, N6-threonylcarbamoyladenosine, 2-methylthio-N6-threonyl carbamoyladenosine, N6-methyl-N6-threonylcarbamoyladenosine, N6-hydroxynorvalylcarbamoyladenosine, 2-methylthio-N6-hydroxynorvalyl carbamoyladenosine, inosine, 3-methylcytidine, 2′-O-methylcytidine, 2-thiocytidine, N4-acetylcytidine, lysidine, 1-methylguanosine, 7-methylguanosine, 2′-O-methylguanosine, queuosine, epoxyqueuosine, 7-cyano-7-deazaguanosine, 7-aminomethyl-7-deazaguanosine, pseudouridine, dihydrouridine, 5-methyluridine, 2′-O-methyluridine, 2-thiouridine, 4-thiouridine, 5-methyl-2-thiouridine, 3-(3-amino-3-carboxypropyl)uridine′, 5-hydroxyuridine, 5-methoxyuridine, uridine 5-oxyacetic acid, uridine 5-oxyacetic acid methyl ester, 5-aminomethyl-2-thiouridine, 5-methylaminomethyluridine, 5-methylaminomethyl-2-thiouridine, 5-methylaminomethyl-2-selenouridine, 5-carboxymethylaminomethyluridine, 5-carboxymethylaminomethyl- 2′-O-methyluridine, 5-carboxymethylaminomethyl-2-thiouridine, 5-(isopentenylaminomethyl)uridine, 5-(isopentenylaminomethyl)- 2-thiouridine, or 5-(isopentenylaminomethyl)- 2-O-methyluridine.

22. Combination of any one of claims 19 to 21, wherein the at least one modified nucleotide is a sugar modified nucleotide, preferably a 2′ ribose modified RNA nucleotide.

23. Combination of claim 22, wherein the 2′ ribose modified RNA nucleotide is a 2′-O-methylated RNA nucleotide.

24. Combination of claim 23, wherein the 2′-O-methylated RNA nucleotide is selected from 2′-O-methylated guanosine (Gm), a 2′-O-methylated uracil (Um), a 2′-O-methylated adenosine (Am), a 2′-O-methylated cytosine (Cm), or a 2′-O-methylated analogue of any of these nucleotides.

25. Combination of any one of claims 16 to 24, wherein the nucleic acid of the second component comprises at least one or more trinucleotide M-X-Y motifs,
wherein M is selected from Gm, Um, or Am, preferably wherein M is Gm;
wherein X is selected from G, A, or U, preferably wherein X is G; and
wherein Y is selected from G, A, U, C, or dihydrouridine, preferably wherein Y is C.

26. Combination of any one of claims 16 to 25, wherein the nucleic acid of the second component comprises or consists of a nucleic acid sequence according to formula I:
N_{W}-M-X—Y—N_{Z} (Formula I)
wherein N is independently selected from G, A, U, C, Gm, Am, Um, Cm, or a modified nucleotide;
wherein W is 0 or an integer of 1 to 15;
wherein Z is 0 or an integer of 1 to 15;
wherein M, X, and Y are selected as defined in claim 25.

27. Combination of any one of claims 16 to 26, wherein the nucleic acid of the second component comprises or consists of at least 2, 3, 4, 5, 6, 7, 8, 9, 10 or more nucleic acid sequences according to formula I, wherein each of the at least 2, 3, 4, 5, 6, 7, 8, 9, 10 or more nucleic acid sequences according to formula I are identical or independently selected from each other.

28. Combination of any one of claims 16 to 27, wherein the nucleic acid of the second component contains a 5′ end that is devoid of a triphosphate group.

29. Combination of any one of claims 16 to 27, wherein the nucleic acid of the second component contains a triphosphate group at the 5′ end.

30. Combination of any one of claims 16 to 29, wherein the nucleic acid of the second component has a length of about 3 to about 50 nucleotides, about 5 to about 25 nucleotides, about 5 to about 15, or about 5 to about 10 nucleotides, preferably about 5 to about 15 nucleotides.

31. Combination of any one of claims 16 to 30, wherein the nucleic acid of the second component has a length of 5 nucleotides, 6 nucleotides, 7 nucleotides, 8 nucleotides, 9 nucleotides, 10 nucleotides, 11 nucleotides, 12 nucleotides, or 13 nucleotides, preferably 9 nucleotides.

32. Combination of any one of claims 16 to 31, wherein the nucleic acid of the second component is a single stranded oligonucleotide.

33. Combination of claim 32, wherein the single stranded oligonucleotide is a single stranded RNA oligonucleotide.

34. Combination of any one of claims 16 to 33, wherein the nucleic acid of the second component, comprises or consists of a nucleic acid sequence derived from a bacterial tRNA, preferably a bacterial tRNA^{Tyr}.

35. Combination of claim 34, wherein the nucleic acid sequence is or is derived from a bacterial tRNA^{Tyr}, preferably from the D-Loop of a bacterial tRNA^{Tyr}, most preferably the D-Loop of Escherichia coli tRNA^{Tyr}.

36. Combination of any one of claims 16 to 35, wherein the nucleic acid of the second component comprises or consists of a nucleic acid sequence identical or at least 70%, 80%, 85%, 86%, 87%, 88%, 89%, 90%, 91%, 92%, 93%, 94%, 95%, 96%, 97%, 98%, or 99% identical to a nucleic acid sequence selected from the group consisting of SEQ ID NOs: 85-212, or fragments of any of these sequences.

37. Combination of claim 36, wherein the nucleic acid of the second component comprises or consists of a nucleic acid sequence identical or at least 70%, 80%, 85%, 86%, 87%, 88%, 89%, 90%, 91%, 92%, 93%, 94%, 95%, 96%, 97%, 98%, or 99% identical to a nucleic acid sequence selected from the group consisting of SEQ ID NOs: 85-87, 149-212, or fragments of any of these sequences, preferably wherein the nucleic acid of the second component comprises or consists of a nucleic acid sequence identical or at least 70%, 80%, 85%, 86%, 87%, 88%, 89%, 90%, 91%, 92%, 93%, 94%, 95%, 96%, 97%, 98%, or 99% identical to a nucleic acid sequence according to 5′-GAG CGmG CCA-3′ (SEQ ID NO: 85), or a fragment thereof.

38. Combination of any one of the preceding claims, wherein the at least one therapeutic RNA of the first component is selected from a coding RNA, a non-coding RNA, a circular RNA (circRNA), an RNA oligonucleotide, a small interfering RNA (siRNA), a small hairpin RNA (shRNA), an antisense RNA (asRNA), a CRISPR/Cas9 guide RNAs, an mRNA, a riboswitch, a ribozyme, an RNA aptamer, a ribosomal RNA (rRNA), a transfer RNA (tRNA), a viral RNA (vRNA), a retroviral RNA, a small nuclear RNA (snRNA), a self-replicating RNA, a replicon RNA, a small nucleolar RNA (snoRNA), a microRNA (miRNA), and a Piwi-interacting RNA (piRNA).

39. Combination of any of the preceding claims, wherein the at least one therapeutic RNA of the first component is an in vitro transcribed RNA.

40. Combination of claim 39, wherein the in vitro transcribed RNA is obtainable by RNA in vitro transcription using a sequence optimized nucleotide mixture.

41. Combination of any of the preceding claims, wherein at least one therapeutic RNA of the first component is a purified RNA.

42. Combination of claim 41, wherein the purified RNA is purified by RP-HPLC and/or TFF and/or Oligo d(T) purification.

43. Combination of any one of the preceding claims, wherein the at least one therapeutic RNA of the first component is a coding RNA.

44. Combination of claim 43, wherein the coding RNA is selected from an mRNA, a self-replicating RNA, a circular RNA, a viral RNA, or a replicon RNA.

45. Combination of any one of the preceding claims, wherein the at least one therapeutic RNA of the first component is an mRNA.

46. Combination of any one of claims 43 to 45, wherein the coding RNA or the mRNA comprises at least one coding sequence encoding at least one peptide or protein.

47. Combination of claim 46, wherein the expression of the encoded at least one peptide or protein of the coding RNA or the mRNA is increased or prolonged by the combination with the at least one antagonist of at least one RNA sensing receptor of the second component upon administration into cells, a tissue or an organism compared to the expression of the encoded at least one peptide or protein of the coding RNA or the mRNA without combination with the at least one antagonist of at least one RNA sensing pattern recognition receptor of the second component.

48. Combination of claim 46 to 47, wherein the at least one peptide or protein is or is derived from a therapeutic peptide or protein.

49. Combination of claim 48, wherein the therapeutic peptide or protein is or is derived from an antibody, an intrabody, a receptor, a receptor agonist, a receptor antagonist, a binding protein, a CRISPR-associated endonuclease, a chaperone, a transporter protein, an ion channel, a membrane protein, a secreted protein, a transcription factor, an enzyme, a peptide or protein hormone, a growth factor, a structural protein, a cytoplasmic protein, a cytoskeletal protein, a viral antigen, a bacterial antigen, a protozoan antigen, an allergen, a tumor antigen, or fragments, variants, or combinations of any of these.

50. Combination of any one of claims 46 to 49, wherein the at least one coding sequence is a codon modified coding sequence, wherein the amino acid sequence encoded by the at least one codon modified coding sequence is preferably not being modified compared to the amino acid sequence encoded by the corresponding wild type coding sequence.

51. Combination of claim 50, wherein the at least one codon modified coding sequence is selected from C maximized coding sequence, CAI maximized coding sequence, human codon usage adapted coding sequence, G/C content modified coding sequence, and G/C optimized coding sequence, or any combination thereof.

52. Combination of any one of the preceding claims, wherein the at least one therapeutic RNA of the first component, preferably the mRNA, comprises a 5′-cap structure.

53. Combination of claim 52, wherein the 5′-cap structure is a cap0, cap1, cap2, a modified cap0 or a modified cap1 structure.

54. Combination of claim 53, wherein the 5′-cap structure is a cap1 structure.

55. Combination of claim 54, wherein the cap1 structure is obtainable by co-transcriptional capping using a trinucleotide cap1 analog.

56. Combination any one of the preceding claims, wherein about 70%, 75%, 80%, 85%, 90%, 95% of the therapeutic RNA (species) of the first component comprises a cap1 structure as determined using a capping detection assay.

57. Combination of any one of the preceding claims, wherein the at least one therapeutic RNA of the first component comprises at least one modified nucleotide or a modified nucleotide analogue.

58. Combination of claim 57, wherein the at least one modified nucleotide is selected from pseudouridine (y), N1-methylpseudouridine (mlL), 5-methylcytosine, and/or 5-methoxyuridine.

59. Combination of any one of the preceding claims, wherein the at least one therapeutic RNA of the first component, preferably the mRNA, comprises at least one poly(A) sequence, and/or at least one poly(C) sequence, and/or at least one histone stem-loop sequence/structure.

60. Combination of claim 59, wherein the poly(A) sequence is located at the 3′ terminus of the therapeutic RNA, and/or wherein the 3′ terminus of the RNA consists of a poly(A) sequence terminating with an A nucleotide.

61. Combination of any one of the preceding claims, wherein the at least one therapeutic RNA of the first component, preferably the mRNA, comprises at least one heterologous 5′-UTR and/or at least one heterologous 3′-UTR.

62. Combination of claim 61, wherein the at least one heterologous 3′-UTR comprises a nucleic acid sequence derived from a 3′-UTR of a gene selected from PSMB3, ALB7, alpha-globin, CASP1, COX6B1, GNAS, NDUFA1 and RPS9, or from a homolog, a fragment or a variant of any one of these genes.

63. Combination of claim 61, wherein the at least one heterologous 5′-UTR comprises a nucleic acid sequence derived from a 5′-UTR of a gene selected from HSD17B4, RPL32, ASAH1, ATP5A1, MP68, NDUFA4, NOSIP, RPL31, SLC7A3, TUBB4B and UBQLN2, or from a homolog, a fragment or variant of any one of these genes.

64. Combination of any one of the preceding claims, wherein the at least one antagonist of the second component, preferably the nucleic acid, and/or the at least one therapeutic RNA of the first component is complexed or associated with or at least partially complexed or partially associated with one or more cationic or polycationic compound, preferably cationic or polycationic polymer, cationic or polycationic polysaccharide, cationic or polycationic lipid, cationic or polycationic protein, or cationic or polycationic peptide, or any combinations thereof.

65. Combination of claim 64, wherein the one or more cationic or polycationic peptides are selected from SEQ ID NO: 39 to 43, or any combinations thereof.

66. Combination of claim 64, wherein the cationic or polycationic polymer is a polyethylene glycol/peptide polymer comprising HO-PEG5000-S-(S—CHHHHHHRRRRHHHHHHC-S-)7-S-PEG5000-OH (SEQ ID NO: 42 of the peptide monomer) and/or a polyethylene glycol/peptide polymer comprising HO-PEG5000-S-(S-CGHHHHHRRRRHHHHHGC-S-)4-S-PEG5000-OH (SEQ ID NO: 43 of the peptide monomer).

67. Combination of claim 65 or 66, additionally comprising a lipid and/or a lipidoid.

68. Combination of any of the proceeding claims, wherein the at least one antagonist of the second component, preferably the nucleic acid, and/or the at least one therapeutic RNA of the first component is complexed, partially complexed, encapsulated, partially encapsulated, or associated with one or more lipids, thereby forming liposomes, lipid nanoparticles (LNP), lipoplexes, and/or nanoliposomes, preferably lipid nanoparticles (LNP).

69. Combination of claim 68, wherein the LNP comprises
(i) at least one cationic lipid, preferably lipid 111-3;
(ii) at least one neutral lipid, preferably 1,2-distearoyl-sn-glycero-3-phosphocholine (DSPC);
(iii) at least one steroid or steroid analogue, preferably cholesterol; and
(iv) at least one PEG-lipid, preferably a PEGylated lipid of formula (IVa),
preferably wherein (i) to (iv) are in a molar ratio of about 20-60% cationic lipid; 5-25% neutral lipid; 25-55% sterol; 0.5-15% PEG-lipid.

70. Pharmaceutical composition comprising or consisting of a combination as defined in any one of claims 1 to 69, and optionally at least one pharmaceutically acceptable carrier.

71. Pharmaceutical composition of claim 70 wherein the at least one therapeutic RNA and the at least one antagonist are formulated separately.

72. Pharmaceutical composition of claim 71 wherein the at least one therapeutic RNA and the at least one antagonist are co-formulated to increase the probability that they are both present in one particle to ensure that the at least one therapeutic RNA and the at least one antagonist are uptaken by the same cell.

73. Pharmaceutical composition of any one of claims 70 to 72, wherein the molar ratio of the at least one antagonist, preferably the nucleic acid, to the at least one therapeutic RNA ranges from about 1:1, to about 100:1, or ranges from about 20:1, to about 80:1.

74. Pharmaceutical composition of any one of claims 70 to 73, wherein the weight to weight ratio of the at least one antagonist, preferably the nucleic acid, to the at least one therapeutic RNA ranges from about 1:1, to about 1:30, or ranges from about 1:2, to about 1:10.

75. Pharmaceutical composition of any one of claims 70 to 74, wherein administration of the composition to a cell, tissue, or organism results in essentially the same or at least a comparable activity of the therapeutic RNA as compared to administration of a corresponding therapeutic RNA only.

76. Pharmaceutical composition of any one of claims 70 to 74, wherein administration of the composition to a cell, tissue, or organism results in increased activity of the therapeutic RNA for example as compared to administration of a corresponding therapeutic RNA only

77. Pharmaceutical composition of claim 75 or 76, wherein activity of the therapeutic RNA is expression of an encoded peptide or protein, preferably protein expression.

78. Pharmaceutical composition of any one of claims 70 to 77, wherein administration of the composition to a cell, tissue, or organism results in a reduced (innate) immune stimulation as compared to administration of the corresponding therapeutic RNA only.

79. Kit or kit of parts comprising at least one first and at least one second component as defined in any one of claims 1 to 69, and/or at least one pharmaceutical composition as defined in any one of claims 70 to 78, optionally comprising a liquid vehicle for solubilising, and, optionally, technical instructions providing information on administration and/or dosage of the components.

80. Combination of any one of claims 1 to 69, pharmaceutical composition of any one of claims 70 to 78, or kit or kit of parts of claim 79 for use as a medicament.

81. Combination of any one of claims 1 to 69, pharmaceutical composition of any one of claims 70 to 78, or kit or kit of parts of claim 79 for use in a chronic medical treatment.

82. Medical use of claim 81, wherein in the chronic medical treatment administration of the combination, the composition, the kit or kit of parts, is performed more than once, for example once or more than once a day, once or more than once a week, once or more than once a month.

83. Combination of any one of claims 1 to 69, pharmaceutical composition of any one of claims 70 to 78, or kit or kit of parts of claim 79 for use in the treatment or prophylaxis of an infection, preferably a virus infection, a bacterial infection, or a protozoan infection.

84. Combination of any one of claims 1 to 69, pharmaceutical composition of any one of claims 70 to 78, or kit or kit of parts of claim 79 for use in the treatment or prophylaxis of a tumour disease, or of a disorder related to such tumour disease.

85. Combination of any one of claims 1 to 69, pharmaceutical composition of any one of claims 70 to 78, or kit or kit of parts of claim 79 for use in the treatment or prophylaxis of a genetic disorder or condition.

86. Combination of any one of claims 1 to 69, pharmaceutical composition of any one of claims 70 to 78, or kit or kit of parts of claim 79 for use in the treatment or prophylaxis of a protein or enzyme deficiency.

87. A method of treating or preventing a disorder, disease, or condition, wherein the method comprises applying or administering to a subject in need thereof the combination as defined in any one of claims 1 to 69, the pharmaceutical composition as defined in any one of claims 70 to 78, or the kit or kit of parts as defined in claim 79.

88. Method of claim 87, wherein administration of the first component and the second component is essentially simultaneous.

89. Method of claim 87, wherein administration of the first component and the second component is sequential.

90. Method of any one of claims 86 to 89, wherein administration of the combination, the pharmaceutical composition, the kit or kit of parts, is performed more than once, for example once or more than once a day, once or more than once a week, once or more than once a month.

91. A method of any one of claims 86 to 90, wherein the administration or applying is subcutaneous, intravenous, intramuscular, intra-articular, intra-synovial, intranasal, oral, intrasternal, intrathecal, intrahepatic, intralesional, intracranial, transdermal, intradermal, intrapulmonal, intraperitoneal, intracardial, intraarterial, intraocular, intravitreal, subretinal, or intratumoral.

92. Method of any one of claims 86 to 91, wherein the subject in need is a mammalian subject, preferably a human subject.

93. Method of reducing the (innate) immune stimulation of a therapeutic RNA wherein the method comprises applying or administering to a subject in need thereof the combination as defined in any one of claims 1 to 69, the pharmaceutical composition as defined in any one of claims 70 to 78, or the kit or kit of parts as defined in claim 79.

94. Method of increasing and/or prolonging the expression of a peptide or protein encoded by a (coding) therapeutic RNA wherein the method comprises applying or administering to a subject in need thereof the combination as defined in any one of claims 1 to 69, the pharmaceutical composition as defined in any one of claims 70 to 78, or the kit or kit of parts as defined in claim 79.


SEQUENCE LISTING (212 sequences)

SEQ ID NO: 1 - DNA, 24 bp, Artificial Sequence
  histone stem-loop sequence
caaaggctcttttcagagccacca

SEQ ID NO: 2 - RNA, 24 nt, Artificial Sequence
  histone stem-loop sequence mRNA
caaaggcucuuuucagagccacca

SEQ ID NO: 3 - DNA, 13 bp, Artificial Sequence
  Kozak
gccgccaccatgg

SEQ ID NO: 4 - RNA, 13 nt, Artificial Sequence
  Kozak mRNA
gccgccaccaugg

SEQ ID NO: 5 - RNA, 6 nt, Artificial Sequence
  mRNA 5′-end
gggaga

SEQ ID NO: 6 - RNA, 6 nt, Artificial Sequence
  mRNA 5′-end
aggaga

SEQ ID NO: 7 - RNA, 128 nt, Artificial Sequence
  mRNA 3′-end A64-N5-C30-histoneSL-N5
aaaaaaaaaaaaaaaaaaaaaaaaaaaaaaaaaaaaaaaaaaaaaaaaaaaaaaaaaaaaaaaaugcaucccccccccccccccccccccccccccccccaaaggcucuuuucagagccaccagaauu

SEQ ID NO: 8 - RNA, 124 nt, Artificial Sequence
  mRNA 3′-end histoneSL-A100
caaaggcucuuuucagagccaccaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 9 - RNA, 100 nt, Artificial Sequence
  mRNA 3′-end A100
aaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 10 - RNA, 134 nt, Artificial Sequence
  mRNA 3′-end N6-A64-N5-C30-histoneSL-N5
auuaauaaaaaaaaaaaaaaaaaaaaaaaaaaaaaaaaaaaaaaaaaaaaaaaaaaaaaaaaaaaaaaaaugcaucccccccccccccccccccccccccccccccaaaggcucuuuucagagccaccagaauu

SEQ ID NO: 11 - RNA, 134 nt, Artificial Sequence
  mRNA 3′-end N6-A64-N5-C30-histoneSL-N5
agaucuaaaaaaaaaaaaaaaaaaaaaaaaaaaaaaaaaaaaaaaaaaaaaaaaaaaaaaaaaaaaaaaaugcaucccccccccccccccccccccccccccccccaaaggcucuuuucagagccaccagaauu

SEQ ID NO: 12 - RNA, 99 nt, Artificial Sequence
  mRNA 3′-end N6-histoneSL-A64-N5
auuaaucaaaggcucuuuucagagccaccaaaaaaaaaaaaaaaaaaaaaaaaaaaaaaaaaaaaaaaaaaaaaaaaaaaaaaaaaaaaaaaaagaauu

SEQ ID NO: 13 - RNA, 99 nt, Artificial Sequence
  mRNA 3′-end N6-histoneSL-A64-N5
agaucucaaaggcucuuuucagagccaccaaaaaaaaaaaaaaaaaaaaaaaaaaaaaaaaaaaaaaaaaaaaaaaaaaaaaaaaaaaaaaaaagaauu

SEQ ID NO: 14 - RNA, 110 nt, Artificial Sequence
  mRNA 3′-end N12-A64-N5-histoneSL-N5
auuaauagaucuaaaaaaaaaaaaaaaaaaaaaaaaaaaaaaaaaaaaaaaaaaaaaaaaaaaaaaaaaaaaaaaaugcaucaaaggcucuuuucagagccaccagaauu

SEQ ID NO: 15 - RNA, 110 nt, Artificial Sequence; identical to SEQ ID NO: 14
  mRNA 3′-end N12-A64-N5-histoneSL-N5

SEQ ID NO: 16 - RNA, 130 nt, Artificial Sequence
  mRNA 3′-end N6-histoneSL-A100
auuaaucaaaggcucuuuucagagccaccaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 17 - RNA, 130 nt, Artificial Sequence
  mRNA 3′-end N6-histoneSL-A100
agaucucaaaggcucuuuucagagccaccaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 18 - RNA, 106 nt, Artificial Sequence
  mRNA 3′-end N6-A100
auuaauaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 19 - RNA, 106 nt, Artificial Sequence
  mRNA 3′-end N6-A100
agaucuaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 20 - RNA, 99 nt, Artificial Sequence
  mRNA 3′-end A75-histoneSL
aaaaaaaaaaaaaaaaaaaaaaaaaaaaaaaaaaaaaaaaaaaaaaaaaaaaaaaaaaaaaaaaaaaaaaaaaaacaaaggcucuuuucagagccacca

SEQ ID NO: 21 - RNA, 102 nt, Artificial Sequence
  mRNA 3′-end U3-A75-histoneSL
uuuaaaaaaaaaaaaaaaaaaaaaaaaaaaaaaaaaaaaaaaaaaaaaaaaaaaaaaaaaaaaaaaaaaaaaaaaaaacaaaggcucuuuucagagccacca

SEQ ID NO: 22 - RNA, 178 nt, Artificial Sequence
  mRNA 3′-end A154-histoneSL
aaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaacaaaggcucuuuucagagccacca

SEQ ID NO: 23 - RNA, 181 nt, Artificial Sequence
  mRNA 3′-end U3-A154-histoneSL
uuuaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaacaaaggcucuuuucagagccacca

SEQ ID NO: 24 - RNA, 101 nt, Artificial Sequence
  mRNA 3′-end N2-A75-histoneSL
agaaaaaaaaaaaaaaaaaaaaaaaaaaaaaaaaaaaaaaaaaaaaaaaaaaaaaaaaaaaaaaaaaaaaaaaaaaacaaaggcucuuuucagagccacca

SEQ ID NO: 25 - RNA, 99 nt, Artificial Sequence
  mRNA 3′-end histoneSL-A75
caaaggcucuuuucagagccaccaaaaaaaaaaaaaaaaaaaaaaaaaaaaaaaaaaaaaaaaaaaaaaaaaaaaaaaaaaaaaaaaaaaaaaaaaaaa

SEQ ID NO: 26 - RNA, 170 nt, Artificial Sequence
  mRNA 3′-end histoneSL-A146
caaaggcucuuuucagagccaccaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 27 - RNA, 136 nt, Artificial Sequence
  mRNA 3′-end N8-A64-N5-C30-histoneSL-N5
agauuaauaaaaaaaaaaaaaaaaaaaaaaaaaaaaaaaaaaaaaaaaaaaaaaaaaaaaaaaaaaaaaaaaugcaucccccccccccccccccccccccccccccccaaaggcucuuuucagagccaccagaauu

SEQ ID NO: 28 - RNA, 131 nt, Artificial Sequence
  mRNA 3′-end N8-A64-N5-C30-histoneSL
agauuaauaaaaaaaaaaaaaaaaaaaaaaaaaaaaaaaaaaaaaaaaaaaaaaaaaaaaaaaaaaaaaaaaugcaucccccccccccccccccccccccccccccccaaaggcucuuuucagagccacca

SEQ ID NO: 29 - RNA, 105 nt, Artificial Sequence
  mRNA 3′-end N8-A64-N5-C4-histoneSL
agauuaauaaaaaaaaaaaaaaaaaaaaaaaaaaaaaaaaaaaaaaaaaaaaaaaaaaaaaaaaaaaaaaaaugcaucccccaaaggcucuuuucagagccacca

SEQ ID NO: 30 - RNA, 115 nt, Artificial Sequence
  mRNA 3′-end N8-A64-N5-C14-histoneSL
agauuaauaaaaaaaaaaaaaaaaaaaaaaaaaaaaaaaaaaaaaaaaaaaaaaaaaaaaaaaaaaaaaaaaugcaucccccccccccccccaaaggcucuuuucagagccacca

SEQ ID NO: 31 - RNA, 129 nt, Artificial Sequence
  mRNA 3′-end N6-A64-N5-C30-histoneSL
agaucuaaaaaaaaaaaaaaaaaaaaaaaaaaaaaaaaaaaaaaaaaaaaaaaaaaaaaaaaaaaaaaaaugcaucccccccccccccccccccccccccccccccaaaggcucuuuucagagccacca

SEQ ID NO: 32 - RNA, 103 nt, Artificial Sequence
  mRNA 3′-end N6-A64-N5-C4-histoneSL
agaucuaaaaaaaaaaaaaaaaaaaaaaaaaaaaaaaaaaaaaaaaaaaaaaaaaaaaaaaaaaaaaaaaugcaucccccaaaggcucuuuucagagccacca

SEQ ID NO: 33 - RNA, 113 nt, Artificial Sequence
  mRNA 3′-end N6-A64-N5-C14-histoneSL
agaucuaaaaaaaaaaaaaaaaaaaaaaaaaaaaaaaaaaaaaaaaaaaaaaaaaaaaaaaaaaaaaaaaugcaucccccccccccccccaaaggcucuuuucagagccacca

SEQ ID NO: 34 - RNA, 99 nt, Artificial Sequence
  mRNA 3′-end N6-A64-histoneSL-N5
agaucuaaaaaaaaaaaaaaaaaaaaaaaaaaaaaaaaaaaaaaaaaaaaaaaaaaaaaaaaaaaaaaaacaaaggcucuuuucagagccaccagaauu

SEQ ID NO: 35 - RNA, 123 nt, Artificial Sequence
  mRNA 3′-end A64-N5-C30-histoneSL
aaaaaaaaaaaaaaaaaaaaaaaaaaaaaaaaaaaaaaaaaaaaaaaaaaaaaaaaaaaaaaaaugcaucccccccccccccccccccccccccccccccaaaggcucuuuucagagccacca

SEQ ID NO: 36 - RNA, 97 nt, Artificial Sequence
  mRNA 3′-end A64-N5-C4-histoneSL
aaaaaaaaaaaaaaaaaaaaaaaaaaaaaaaaaaaaaaaaaaaaaaaaaaaaaaaaaaaaaaaaugcaucccccaaaggcucuuuucagagccacca

SEQ ID NO: 37 - RNA, 107 nt, Artificial Sequence
  mRNA 3′-end A64-N5-C14-histoneSL
aaaaaaaaaaaaaaaaaaaaaaaaaaaaaaaaaaaaaaaaaaaaaaaaaaaaaaaaaaaaaaaaugcaucccccccccccccccaaaggcucuuuucagagccacca

SEQ ID NO: 38 - RNA, 93 nt, Artificial Sequence
  mRNA 3′-end A64-histoneSL-N5
aaaaaaaaaaaaaaaaaaaaaaaaaaaaaaaaaaaaaaaaaaaaaaaaaaaaaaaaaaaaaaaacaaaggcucuuuucagagccaccagaauu

SEQ ID NO: 39 - protein, 14 aa, Artificial Sequence
  carrier peptide 1
CRRRRRRRRRRRRC

SEQ ID NO: 40 - protein, 13 aa, Artificial Sequence
  carrier peptide 2
CRRRRRRRRRRRR

SEQ ID NO: 41 - protein, 14 aa, Artificial Sequence
  carrier peptide 3
WRRRRRRRRRRRRC

SEQ ID NO: 42 - protein, 18 aa, Artificial Sequence
  carrier peptide 4
CHHHHHHRRRRHHHHHHC

SEQ ID NO: 43 - protein, 18 aa, Artificial Sequence
  carrier peptide 5
CGHHHHHRRRRHHHHHGC

SEQ ID NO: 44 - DNA, 62 bp, Artificial Sequence
  HSD17B4 5′-UTR
gtcccgcagtcggcgtccagcggctctgcttgttcgtgtgtgtgtcgttgcaggccttattc

SEQ ID NO: 45 - RNA, 62 nt, Artificial Sequence
  HSD17B4 5′-UTR
gucccgcagucggcguccagcggcucugcuuguucgugugugugucguugcaggccuuauuc

SEQ ID NO: 46 - DNA, 42 bp, Artificial Sequence
  ASAH1 5′-UTR
gcctctgctggagtccggggagtggcgttggctgctagagcg

SEQ ID NO: 47 - RNA, 42 nt, Artificial Sequence
  ASAH1 5′-UTR
gccucugcuggaguccggggaguggcguuggcugcuagagcg

SEQ ID NO: 48 - DNA, 75 bp, Artificial Sequence
  ATP5A1 5′-UTR
gcggctcggccattttgtcccagtcagtccggaggctgcggctgcagaagtaccgcctgcggagtaactgcaaag

SEQ ID NO: 49 - RNA, 75 nt, Artificial Sequence
  ATP5A1 5′-UTR
gcggcucggccauuuugucccagucaguccggaggcugcggcugcagaaguaccgccugcggaguaacugcaaag

SEQ ID NO: 50 - DNA, 54 bp, Artificial Sequence
  Mp68_(2010107E04Rik) 5′-UTR
ctttcccattctgtagcagaatttggtgttgcctgtggtcttggtcccgcggag

SEQ ID NO: 51 - RNA, 54 nt, Artificial Sequence
  Mp68_(2010107E04Rik) 5′-UTR
cuuucccauucuguagcagaauuugguguugccuguggucuuggucccgcggag

SEQ ID NO: 52 - DNA, 81 bp, Artificial Sequence
  Ndufa4 5′-UTR
gtccgctcagccaggttgcagaagcggcttagcgtgtgtcctaatcttctctctgcgtgtaggtaggcctgtgccgcaaac

SEQ ID NO: 53 - RNA, 81 nt, Artificial Sequence
  Ndufa4 5′-UTR
guccgcucagccagguugcagaagcggcuuagcguguguccuaaucuucucucugcguguagguaggccugugccgcaaac

SEQ ID NO: 54 - DNA, 108 bp, Artificial Sequence
  Nosip 5′-UTR
ctcctgtcgggcggaagtaggaggagtagagtttaaaaacagtactctttttccggttcgggacgtagttgaagcaacgacaagccggataaccgctcttgagacagg

SEQ ID NO: 55 - RNA, 108 nt, Artificial Sequence
  Nosip 5′-UTR
cuccugucgggcggaaguaggaggaguagaguuuaaaaacaguacucuuuuuccgguucgggacguaguugaagcaacgacaagccggauaaccgcucuugagacagg

SEQ ID NO: 56 - DNA, 84 bp, Artificial Sequence
  Rpl31 5′-UTR
cccgtgacccggaagttgtacggctacgcgactttccctcccacaaaccctcgcgcccttcctttcctacttgggcccggcaga

SEQ ID NO: 57 - RNA, 84 nt, Artificial Sequence
  Rpl31 5′-UTR
cccgugacccggaaguuguacggcuacgcgacuuucccucccacaaacccucgcgcccuuccuuuccuacuugggcccggcaga

SEQ ID NO: 58 - DNA, 159 bp, Artificial Sequence
  Slc7a3 5′-UTR
gggcgcttggcttgcaaggaccctgagctgcggcattgaagcacacccaacccaactcgactgaagtcagcctcactgaaccggatctgagaatcttctctctctgggcttgccagggctctccgaacctagctagcatcctcttcaattccaactaga

SEQ ID NO: 59 - RNA, 159 nt, Artificial Sequence
  Slc7a3 5′-UTR
gggcgcuuggcuugcaaggacccugagcugcggcauugaagcacacccaacccaacucgacugaagucagccucacugaaccggaucugagaaucuucucucucugggcuugccagggcucuccgaaccuagcuagcauccucuucaauuccaacuaga

SEQ ID NO: 60 - DNA, 102 bp, Artificial Sequence
  TUBB4B 5′-UTR
atataagcgttggcggagcgtcggttgtagcactctgcgcgcccgctcttctgctgctgtttgtctacttcctcctgcttccccgccgccgccgccgccatc

SEQ ID NO: 61 - RNA, 102 nt, Artificial Sequence
  TUBB4B 5′-UTR
auauaagcguuggcggagcgucgguuguagcacucugcgcgcccgcucuucugcugcuguuugucuacuuccuccugcuuccccgccgccgccgccgccauc

SEQ ID NO: 62 - DNA, 222 bp, Artificial Sequence
  Ubqln2 5′-UTR
cggagacggcctgcaggacctgctctctcagccctcagccgaggcctacgccgagccgagtgcgcagccgacgaccgggaggagccgcagccttcaactctgaggtactgtgatccgcgctgcccgccgggccgccccagtccgctgctgcggcacctccttccctcgcgccctcttcgctcgccagcgccttccctgtgagcctgcgtcaccgcggccgcc

SEQ ID NO: 63 - RNA, 222 nt, Artificial Sequence
  Ubqln2 5′-UTR
cggagacggccugcaggaccugcucucucagcccucagccgaggccuacgccgagccgagugcgcagccgacgaccgggaggagccgcagccuucaacucugagguacugugauccgcgcugcccgccgggccgccccaguccgcugcugcggcaccuccuucccucgcgcccucuucgcucgccagcgccuucccugugagccugcgucaccgcggccgcc

SEQ ID NO: 64 - DNA, 42 bp, Artificial Sequence
  RPL32 (32L4, 32L3) 5′-UTR
ggcgctgcctacggaggtggcagccatctccttctcggcatc

SEQ ID NO: 65 - RNA, 42 nt, Artificial Sequence
  RPL32 (32L4, 32L3) 5′-UTR
ggcgcugccuacggagguggcagccaucuccuucucggcauc

SEQ ID NO: 66 - DNA, 57 bp, Artificial Sequence
  PSMB3 3′-UTR
ccctgttcccagagcccacttttttttctttttttgaaataaaatagcctgtctttc

SEQ ID NO: 67 - RNA, 57 nt, Artificial Sequence
  PSMB3 3′-UTR
cccuguucccagagcccacuuuuuuuucuuuuuuugaaauaaaauagccugucuuuc

SEQ ID NO: 68 - DNA, 121 bp, Artificial Sequence
  CASP1 3′-UTR
aataaggaaactgtatgaatgtctgtgggcaggaagtgaagagatccttctgtaaaggtttttggaattatgtctgctgaataataaacttttttgaaataataaatctggtagaaaaatg

SEQ ID NO: 69 - RNA, 121 nt, Artificial Sequence
  CASP1 3′-UTR
aauaaggaaacuguaugaaugucugugggcaggaagugaagagauccuucuguaaagguuuuuggaauuaugucugcugaauaauaaacuuuuuugaaauaauaaaucugguagaaaaaug

SEQ ID NO: 70 - DNA, 137 bp, Artificial Sequence
  COX6B1 3′-UTR
actggctgcatctccctttcctctgtcctccatccttctcccaggatggtgaagggggacctggtacccagtgatccccaccccaggatcctaaatcatgacttacctgctaataaaaactcattggaaaagtgaga

SEQ ID NO: 71 - RNA, 137 nt, Artificial Sequence
  COX6B1 3′-UTR
acuggcugcaucucccuuuccucuguccuccauccuucucccaggauggugaagggggaccugguacccagugauccccaccccaggauccuaaaucaugacuuaccugcuaauaaaaacucauuggaaaagugaga

SEQ ID NO: 72 - DNA, 353 bp, Artificial Sequence
  Gnas 3′-UTR
gaagggaacacccaaatttaattcagccttaagcacaattaattaagagtgaaacgtaattgtacaagcagttggtcacccaccatagggcatgatcaacaccgcaacctttcctttttcccccagtgattctgaaaaacccctcttcccttcagcttgcttagatgttccaaatttagtaagcttaaggcggcctacagaagaaaaagaaaaaaaaggccacaaaagttccctctcactttcagtaaataaaataaaagcagcaacagaaataaagaaataaatgaaattcaaaatgaaataaatattgtgttgtgcagcattaaaaaatcaataaaaattaaaaatgagca

SEQ ID NO: 73 - RNA, 353 nt, Artificial Sequence
  Gnas 3′-UTR
gaagggaacacccaaauuuaauucagccuuaagcacaauuaauuaagagugaaacguaauuguacaagcaguuggucacccaccauagggcaugaucaacaccgcaaccuuuccuuuuucccccagugauucugaaaaaccccucuucccuucagcuugcuuagauguuccaaauuuaguaagcuuaaggcggccuacagaagaaaaagaaaaaaaaggccacaaaaguucccucucacuuucaguaaauaaaauaaaagcagcaacagaaauaaagaaauaaaugaaauucaaaaugaaauaaauauuguguugugcagcauuaaaaaaucaauaaaaauuaaaaaugagca

SEQ ID NO: 74 - DNA, 133 bp, Artificial Sequence
  Ndufa1 3′-UTR
ggaagcattttcctggctgattaaaagaaattactcagctatggtcatctgttcctgttagaaggctatgcagcatattatatactatgcgcatgttatgaaatgcataataaaaaattttaaaaaatctaaa

SEQ ID NO: 75 - RNA, 133 nt, Artificial Sequence
  Ndufa1 3′-UTR
ggaagcauuuuccuggcugauuaaaagaaauuacucagcuauggucaucuguuccuguuagaaggcuaugcagcauauuauauacuaugcgcauguuaugaaaugcauaauaaaaaauuuuaaaaaaucuaaa

SEQ ID NO: 76 - DNA, 70 bp, Artificial Sequence
  RPS9 3′-UTR
gtccacctgtccctcctgggctgctggattgtctcgttttcctgccaaataaacaggatcagcgctttac

SEQ ID NO: 77 - RNA, 70 nt, Artificial Sequence
  RPS9 3′-UTR
guccaccugucccuccugggcugcuggauugucucguuuuccugccaaauaaacaggaucagcgcuuuac

SEQ ID NO: 78 - DNA, 187 bp, Artificial Sequence
  ALB7 3′-UTR
gcatcacatttaaaagcatctcagcctaccatgagaataagagaaagaaaatgaagatcaatagcttattcatctctttttctttttcgttggtgtaaagccaacaccctgtctaaaaaacataaatttctttaatcattttgcctcttttctctgtgcttcaattaataaaaaatggaaagaacct

SEQ ID NO: 79 - RNA, 187 nt, Artificial Sequence
  ALB7 3′-UTR
gcaucacauuuaaaagcaucucagccuaccaugagaauaagagaaagaaaaugaagaucaauagcuuauucaucucuuuuucuuuuucguugguguaaagccaacacccugucuaaaaaacauaaauuucuuuaaucauuuugccucuuuucucugugcuucaauuaauaaaaaauggaaagaaccu

SEQ ID NO: 80 - DNA, 44 bp, Artificial Sequence
  muag 3′-UTR
gcccgatgggcctcccaacgggccctcctcccctccttgcaccg

SEQ ID NO: 81 - RNA, 44 nt, Artificial Sequence
  muag 3′-UTR
gcccgaugggccucccaacgggcccuccuccccuccuugcaccg

SEQ ID NO: 82 - RNA, 2035 nt, Artificial Sequence
  PpLuc
ggggcgcugccuacggagguggcagccaucuccuucucggcaucaagcuugaggauggaggacgccaagaacaucaagaagggcccggcgcccuucuacccgcuggaggacgggaccgccggcgagcagcuccacaaggccaugaagcgguacgcccuggugccgggcacgaucgccuucaccgacgcccacaucgaggucgacaucaccuacgcggaguacuucgagaugagcgugcgccuggccgaggccaugaagcgguacggccugaacaccaaccaccggaucguggugugcucggagaacagccugcaguucuucaugccggugcugggcgcccucuucaucggcguggccgucgccccggcgaacgacaucuacaacgagcgggagcugcugaacagcauggggaucagccagccgaccgugguguucgugagcaagaagggccugcagaagauccugaacgugcagaagaagcugcccaucauccagaagaucaucaucauggacagcaagaccgacuaccagggcuuccagucgauguacacguucgugaccagccaccucccgccgggcuucaacgaguacgacuucgucccggagagcuucgaccgggacaagaccaucgcccugaucaugaacagcagcggcagcaccggccugccgaaggggguggcccugccgcaccggaccgccugcgugcgcuucucgcacgcccgggaccccaucuucggcaaccagaucaucccggacaccgccauccugagcguggugccguuccaccacggcuucggcauguucacgacccugggcuaccucaucugcggcuuccgggugguccugauguaccgguucgaggaggagcuguuccugcggagccugcaggacuacaagauccagagcgcgcugcucgugccgacccuguucagcuucuucgccaagagcacccugaucgacaaguacgaccugucgaaccugcacgagaucgccagcgggggcgccccgcugagcaaggaggugggcgaggccguggccaagcgguuccaccucccgggcauccgccagggcuacggccugaccgagaccacgagcgcgauccugaucacccccgagggggacgacaagccgggcgccgugggcaagguggucccguucuucgaggccaaggugguggaccuggacaccggcaagacccugggcgugaaccagcggggcgagcugugcgugcgggggccgaugaucaugagcggcuacgugaacaacccggaggccaccaacgcccucaucgacaaggacggcuggcugcacagcggcgacaucgccuacugggacgaggacgagcacuucuucaucgucgaccggcugaagucgcugaucaaguacaagggcuaccagguggcgccggccgagcuggagagcauccugcuccagcaccccaacaucuucgacgccggcguggccgggcugccggacgacgacgccggcgagcugccggccgcggugguggugcuggagcacggcaagaccaugacggagaaggagaucgucgacuacguggccagccaggugaccaccgccaagaagcugcggggcggcgugguguucguggacgaggucccgaagggccugaccgggaagcucgacgcccggaagauccgcgagauccugaucaaggccaagaagggcggcaagaucgccguguaagacuagugcaucacauuuaaaagcaucucagccuaccaugagaauaagagaaagaaaaugaagaucaauagcuuauucaucucuuuuucuuuuucguugguguaaagccaacacccugucuaaaaaacauaaauuucuuuaaucauuuugccucuuuucucugugcuucaauuaauaaaaaauggaaagaaccuagaucuaaaaaaaaaaaaaaaaaaaaaaaaaaaaaaaaaaaaaaaaaaaaaaaaaaaaaaaaaaaaaaaaugcaucccccccccccccccccccccccccccccccaaaggcucuuuucagagccaccagaauu

SEQ ID NO: 83 - RNA, 1929 nt, Artificial Sequence
  PpLuc
aggagagucccgcagucggcguccagcggcucugcuuguucgugugugugucguugcaggccuuauucaagcuuaccauggaggacgccaagaacaucaagaagggcccggcgcccuucuacccgcuggaggacgggaccgccggcgagcagcuccacaaggccaugaagcgguacgcccuggugccgggcacgaucgccuucaccgacgcccacaucgaggucgacaucaccuacgcggaguacuucgagaugagcgugcgccuggccgaggccaugaagcgguacggccugaacaccaaccaccggaucguggugugcucggagaacagccugcaguucuucaugccggugcugggcgcccucuucaucggcguggccgucgccccggcgaacgacaucuacaacgagcgggagcugcugaacagcauggggaucagccagccgaccgugguguucgugagcaagaagggccugcagaagauccugaacgugcagaagaagcugcccaucauccagaagaucaucaucauggacagcaagaccgacuaccagggcuuccagucgauguacacguucgugaccagccaccucccgccgggcuucaacgaguacgacuucgucccggagagcuucgaccgggacaagaccaucgcccugaucaugaacagcagcggcagcaccggccugccgaaggggguggcccugccgcaccggaccgccugcgugcgcuucucgcacgcccgggaccccaucuucggcaaccagaucaucccggacaccgccauccugagcguggugccguuccaccacggcuucggcauguucacgacccugggcuaccucaucugcggcuuccgggugguccugauguaccgguucgaggaggagcuguuccugcggagccugcaggacuacaagauccagagcgcgcugcucgugccgacccuguucagcuucuucgccaagagcacccugaucgacaaguacgaccugucgaaccugcacgagaucgccagcgggggcgccccgcugagcaaggaggugggcgaggccguggccaagcgguuccaccucccgggcauccgccagggcuacggccugaccgagaccacgagcgcgauccugaucacccccgagggggacgacaagccgggcgccgugggcaagguggucccguucuucgaggccaaggugguggaccuggacaccggcaagacccugggcgugaaccagcggggcgagcugugcgugcgggggccgaugaucaugagcggcuacgugaacaacccggaggccaccaacgcccucaucgacaaggacggcuggcugcacagcggcgacaucgccuacugggacgaggacgagcacuucuucaucgucgaccggcugaagucgcugaucaaguacaagggcuaccagguggcgccggccgagcuggagagcauccugcuccagcaccccaacaucuucgacgccggcguggccgggcugccggacgacgacgccggcgagcugccggccgcggugguggugcuggagcacggcaagaccaugacggagaaggagaucgucgacuacguggccagccaggugaccaccgccaagaagcugcggggcggcgugguguucguggacgaggucccgaagggccugaccgggaagcucgacgcccggaagauccgcgagauccugaucaaggccaagaagggcggcaagaucgccgugugaggacuagucccuguucccagagcccacuuuuuuuucuuuuuuugaaauaaaauagccugucuuucagaucuaaaaaaaaaaaaaaaaaaaaaaaaaaaaaaaaaaaaaaaaaaaaaaaaaaaaaaaaaaaaaaaaugcaucccccccccccccccccccccccccccccccaaaggcucuuuucagagccaccagaauu

SEQ ID NO: 84 - RNA, 547 nt, Artificial Sequence
  Immune stimulatory RNA
gggagaaagcucaagcuuauccaaguaggcuggucaccuguacaacguagccgguauuuuuuuuuuuuuuuuuuuuuugaccgucucaagguccaaguuagucugccuauaaaggugcggauccacagcugaugaaagacuugugcgguacgguuaaucuccccuuuuuuuuuuuuuuuuuuuuuaguaaaugcgucuacugaauccagcgaugaugcuggcccagaucuucgaccacaagugcauauaguagucaucgagggucgccuuuuuuuuuuuuuuuuuuuuuuuggcccaguucugagacuucgcuagagacuacaguuacagcugcaguaguaaccacugcggcuauugcaggaaaucccguucagguuuuuuuuuuuuuuuuuuuuuccgcucacuaugauuaagaaccagguggagugucacugcucucgaggucucacgagagcgcucgauacaguccuuggaagaaucuuuuuuuuuuuuuuuuuuuuuugugcgacgaucacagagaacuucuauucaugcaggucugcucuag

SEQ ID NO: 85 - RNA, 9 nt, Artificial Sequence
  9mer oligonucleotide
  modified_base (5)..(5): n can be any nucleotide, wherein the nucleotide is 2-O-methylated, preferably a 2-O-methylated guanosine
gagcngcca

SEQ ID NO: 86 - RNA, 7 nt, Artificial Sequence
  7mer oligonucleotide
  modified_base (4)..(4): n can be any nucleotide, wherein the nucleotide is 2-O-methylated, preferably a 2-O-methylated guanosine
agcngcc

SEQ ID NO: 87 - RNA, 5 nt, Artificial Sequence
  5mer oligonucleotide
  modified_base (3)..(3): n can be any nucleotide, wherein the nucleotide is 2-O-methylated, preferably a 2-O-methylated guanosine
gcngc

SEQ ID NO: 88 - RNA, 26 nt, Artificial Sequence
  26mer oligonucleotide
  modified_base (16)..(16): n can be any nucleotide, wherein the nucleotide is 2-O-methylated, preferably a 2-O-methylated guanosine
gugggguucccgagcngccaaaggga

SEQ ID NO: 89 - RNA, 26 nt, Artificial Sequence
  26mer oligonucleotide 17 A
  modified_base (16)..(16): n can be any nucleotide, wherein the nucleotide is 2-O-methylated, preferably a 2-O-methylated guanosine
gugggguucccgagangccaaaggga

SEQ ID NO: 90 - RNA, 26 nt, Artificial Sequence
  26mer oligonucleotide 17 G
  modified_base (16)..(16): n can be any nucleotide, wherein the nucleotide is 2-O-methylated, preferably a 2-O-methylated guanosine
gugggguucccgaggngccaaaggga

SEQ ID NO: 91 - RNA, 26 nt, Artificial Sequence
  26mer oligonucleotide 17 U
  modified_base (16)..(16): n can be any nucleotide, wherein the nucleotide is 2-O-methylated, preferably a 2-O-methylated guanosine
gugggguucccgagungccaaaggga

SEQ ID NO: 92 - RNA, 26 nt, Artificial Sequence
  26mer oligonucleotide 17 A
  modified_base (16)..(16): n can be any nucleotide, wherein the nucleotide is 2-O-methylated, preferably a 2-O-methylated adenosine
gugggguucccgagcngccaaaggga

SEQ ID NO: 93 - RNA, 26 nt, Artificial Sequence
  26mer oligonucleotide 17 C
  modified_base (16)..(16): n can be any nucleotide, wherein the nucleotide is 2-O-methylated, preferably a 2-O-methylated cytosine
gugggguucccgagcngccaaaggga

SEQ ID NO: 94 - RNA, 26 nt, Artificial Sequence
  26mer oligonucleotide 17 U
  modified_base (16)..(16): n can be any nucleotide, wherein the nucleotide is 2-O-methylated, preferably a 2-O-methylated uracil
gugggguucccgagcngccaaaggga

SEQ ID NO: 95 - RNA, 26 nt, Artificial Sequence
  26mer oligonucleotide 19 A
  modified_base (16)..(16): n can be any nucleotide, wherein the nucleotide is 2-O-methylated, preferably a 2-O-methylated guanosine
gugggguucccgagcnaccaaaggga

SEQ ID NO: 96 - RNA, 26 nt, Artificial Sequence
  26mer oligonucleotide 19 C
  modified_base (16)..(16): n can be any nucleotide, wherein the nucleotide is 2-O-methylated, preferably a 2-O-methylated guanosine
gugggguucccgagcncccaaaggga

SEQ ID NO: 97 - RNA, 26 nt, Artificial Sequence
  26mer oligonucleotide 19 U
  modified_base (16)..(16): n can be any nucleotide, wherein the nucleotide is 2-O-methylated, preferably a 2-O-methylated guanosine
gugggguucccgagcnuccaaaggga

SEQ ID NO: 98 - RNA, 26 nt, Artificial Sequence
  26mer oligonucleotide 20 A
  modified_base (16)..(16): n can be any nucleotide, wherein the nucleotide is 2-O-methylated, preferably a 2-O-methylated guanosine
gugggguucccgagcngacaaaggga

SEQ ID NO: 99 - RNA, 26 nt, Artificial Sequence
  26mer oligonucleotide 20 G
  modified_base (16)..(16): n can be any nucleotide, wherein the nucleotide is 2-O-methylated, preferably a 2-O-methylated guanosine
gugggguucccgagcnggcaaaggga

SEQ ID NO: 100 - RNA, 26 nt, Artificial Sequence
  26mer oligonucleotide 20 U
  modified_base (16)..(16): n can be any nucleotide, wherein the nucleotide is 2-O-methylated, preferably a 2-O-methylated guanosine
gugggguucccgagcngucaaaggga

SEQ ID NO: 101 - RNA, 21 nt, Artificial Sequence
  gal-mU
  modified_base (2)..(2): n can be any nucleotide, wherein the nucleotide is 2-O-methylated, preferably a 2-O-methylated uracil
  modified_base (10)..(10): n can be any nucleotide, wherein the nucleotide is 2-O-methylated, preferably a 2-O-methylated uracil
  modified_base (17)..(19): n can be any nucleotide, wherein the nucleotide is 2-O-methylated, preferably a 2-O-methylated uracil
cnacacaaancagcgannnuu

SEQ ID NO: 102 - RNA, 21 nt, Artificial Sequence
  gal-mC
  modified_base (1)..(1): n can be any nucleotide, wherein the nucleotide is 2-O-methylated, preferably a 2-O-methylated cytosine
  modified_base (4)..(4): n can be any nucleotide, wherein the nucleotide is 2-O-methylated, preferably a 2-O-methylated cytosine
  modified_base (6)..(6): n can be any nucleotide, wherein the nucleotide is 2-O-methylated, preferably a 2-O-methylated cytosine
  modified_base (11)..(11): n can be any nucleotide, wherein the nucleotide is 2-O-methylated, preferably a 2-O-methylated cytosine
  modified_base (14)..(14): n can be any nucleotide, wherein the nucleotide is 2-O-methylated, preferably a 2-O-methylated cytosine
nuananaaaunagngauuuuu

SEQ ID NO: 103 - RNA, 21 nt, Artificial Sequence
  gal-mG
  modified_base (13)..(13): n can be any nucleotide, wherein the nucleotide is 2-O-methylated, preferably a 2-O-methylated guanosine
  modified_base (15)..(15): n can be any nucleotide, wherein the nucleotide is 2-O-methylated, preferably a 2-O-methylated guanosine
cuacacaaaucancnauuuuu

SEQ ID NO: 104 - RNA, 21 nt, Artificial Sequence
  gal-mA
  modified_base (3)..(3): n can be any nucleotide, wherein the nucleotide is 2-O-methylated, preferably a 2-O-methylated adenosine
  modified_base (5)..(5): n can be any nucleotide, wherein the nucleotide is 2-O-methylated, preferably a 2-O-methylated adenosine
  modified_base (7)..(9): n can be any nucleotide, wherein the nucleotide is 2-O-methylated, preferably a 2-O-methylated adenosine
  modified_base (12)..(12): n can be any nucleotide, wherein the nucleotide is 2-O-methylated, preferably a 2-O-methylated adenosine
  modified_base (16)..(16): n can be any nucleotide, wherein the nucleotide is 2-O-methylated, preferably a 2-O-methylated adenosine
cuncncnnnucngcgnuuuuu

SEQ ID NO: 105 - RNA, 21 nt, Artificial Sequence
  NP-mU
  modified_base (4)..(4): n can be any nucleotide, wherein the nucleotide is 2-O-methylated, preferably a 2-O-methylated uracil
  modified_base (6)..(7): n can be any nucleotide, wherein the nucleotide is 2-O-methylated, preferably a 2-O-methylated uracil
  modified_base (9)..(11): n can be any nucleotide, wherein the nucleotide is 2-O-methylated, preferably a 2-O-methylated uracil
  modified_base (13)..(14): n can be any nucleotide, wherein the nucleotide is 2-O-methylated, preferably a 2-O-methylated uracil
ggancnnannncnncggaguu

SEQ ID NO: 106 - RNA, 21 nt, Artificial Sequence
  NP-mC
  modified_base (5)..(5): n can be any nucleotide, wherein the nucleotide is 2-O-methylated, preferably a 2-O-methylated cytosine
  modified_base (12)..(12): n can be any nucleotide, wherein the nucleotide is 2-O-methylated, preferably a 2-O-methylated cytosine
  modified_base (15)..(15): n can be any nucleotide, wherein the nucleotide is 2-O-methylated, preferably a 2-O-methylated cytosine
ggaunuuauuunuunggaguu

SEQ ID NO: 107 - RNA, 21 nt, Artificial Sequence
  Luc-mU
  modified_base (3)..(4): n can be any nucleotide, wherein the nucleotide is 2-O-methylated, preferably a 2-O-methylated uracil
  modified_base (6)..(6): n can be any nucleotide, wherein the nucleotide is 2-O-methylated, preferably a 2-O-methylated uracil
  modified_base (8)..(8): n can be any nucleotide, wherein the nucleotide is 2-O-methylated, preferably a 2-O-methylated uracil
  modified_base (13)..(14): n can be any nucleotide, wherein the nucleotide is 2-O-methylated, preferably a 2-O-methylated uracil
  modified_base (16)..(16): n can be any nucleotide, wherein the nucleotide is 2-O-methylated, preferably a 2-O-methylated uracil
  modified_base (18)..(18): n can be any nucleotide, wherein the nucleotide is 2-O-methylated, preferably a 2-O-methylated uracil
gannangnccggnnangnauu

SEQ ID NO: 108 - DNA, 20 bp, Artificial Sequence
  ODN 2216 (PO)
gggggacgatcgtcgggggg

SEQ ID NO: 109 - DNA, 25 bp, Artificial Sequence
  ODN M362 (PO)
tcgtcgtcgttcgaacgacgttgat

SEQ ID NO: 110 - DNA, 16 bp, Artificial Sequence
  ODN 6295 (PO)
taacgttgaggggcat

SEQ ID NO: 111 - DNA, 20 bp, Artificial Sequence
  ODN 1826 (PO)
tccatgacgttcctgacgtt

SEQ ID NO: 112 - RNA, 18 nt, Artificial Sequence
  IRS-954 (DV-1079)
ugcuccuggagggguugu

SEQ ID NO: 113 - RNA, 18 nt, Artificial Sequence
  IRO-5
cuaucugacguucucugu

SEQ ID NO: 114 - RNA, 15 nt, Artificial Sequence
  IRS 2088
uccuggcggggaagu

SEQ ID NO: 115 - RNA, 15 nt, Artificial Sequence
  IRS 869
uccuggagggguugu

SEQ ID NO: 116 - RNA, 15 nt, Artificial Sequence
  INH-ODN-2114
uccuggaggggaagu

SEQ ID NO: 117 - RNA, 15 nt, Artificial Sequence
  INH-ODN 4024
uccuggaugggaagu

SEQ ID NO: 118 - RNA, 12 nt, Artificial Sequence
  INH-ODN 4084-F
ccuggaugggaa

SEQ ID NO: 119 - RNA, 20 nt, Artificial Sequence
  IRS-661
ugcuugcaagcuugcaagca

SEQ ID NO: 120 - RNA, 18 nt, Artificial Sequence
  IRS-954
ugcuccuggagggguugu

SEQ ID NO: 121 - RNA, 15 nt, Artificial Sequence
  INH-ODN-24888
uccuggcggggaagu

SEQ ID NO: 122 - RNA, 15 nt, Artificial Sequence
  IHN-ODN 2088
uccuggcggggaagu

SEQ ID NO: 123 - RNA, 24 nt, Artificial Sequence
  ODN A151
uuaggguuaggguuaggguuaggg

SEQ ID NO: 124 - RNA, 21 nt, Artificial Sequence
  G-ODN
cuccuauuggggguuuccuau

SEQ ID NO: 125 - RNA, 24 nt, Artificial Sequence
  ODN INH-1
ccuggaugggaauucccauccagg

SEQ ID NO: 126 - RNA, 24 nt, Artificial Sequence
  ODN INH-18
ccuggaugggaacuuaccgcugca

SEQ ID NO: 127 - RNA, 24 nt, Artificial Sequence
  INH-4
uucccauccaggccuggaugggaa

SEQ ID NO: 128 - RNA, 24 nt, Artificial Sequence
  INH-13
cuuaccgcugcaccuggaugggaa

SEQ ID NO: 129 - RNA, 24 nt, Artificial Sequence
  (pS-) ST-ODN
ucgucguuuugucguuuugucguu

SEQ ID NO: 130 - RNA, 15 nt, Artificial Sequence
  INH-ODN 21 14
uccuggaggggaagu

SEQ ID NO: 131 - RNA, 15 nt, Artificial Sequence
  Endosomal TLR Targets TLR7
uccuggagggguugu

SEQ ID NO: 132 - RNA, 20 nt, Artificial Sequence
  Endosomal TLR Targets TLR9
ugcuugcaagcuugcaagca

SEQ ID NO: 133 - RNA, 18 nt, Artificial Sequence
  Endosomal TLR Targets TLR7 and 9
ugcuccuggagggguugu

SEQ ID NO: 134 - RNA, 12 nt, Artificial Sequence
  modified ORN 1
  modified_base (2)..(2): n can be any nucleotide, wherein the nucleotide is 2-O-methylated, preferably a 2-O-methylated adenosine
gnuacuuaccug

SEQ ID NO: 135 - RNA, 16 nt, Artificial Sequence
  modified ORN 2
  modified_base (8)..(8): n can be any nucleotide, wherein the nucleotide is 2-O-methylated, preferably a 2-O-methylated guanosine
ccgagccnaaggcacc

SEQ ID NO: 136 - RNA, 12 nt, Artificial Sequence
  modified ORN 3
  modified_base (1)..(1): n can be any nucleotide, wherein the nucleotide is 2-O-methylated, preferably a 2-O-methylated guanosine
  modified_base (2)..(2): n can be any nucleotide, wherein the nucleotide is 2-O-methylated, preferably a 2-O-methylated adenosine
nnuacuuaccug

SEQ ID NO: 137 - RNA, 12 nt, Artificial Sequence
  modified ORN 4
  modified_base (1)..(1): n can be any nucleotide, wherein the nucleotide is 2-O-methylated, preferably a 2-O-methylated guanosine
nauacuuaccug

SEQ ID NO: 138 - RNA, 16 nt, Artificial Sequence
  modified ORN 5
  modified_base (4)..(4): n can be any nucleotide, wherein the nucleotide is 2-O-methylated, preferably a 2-O-methylated adenosine
ccgngccgauuguacc

SEQ ID NO: 139 - RNA, 16 nt, Artificial Sequence
  modified ORN 6
  modified_base (9)..(9): n can be any nucleotide, wherein the nucleotide is 2-O-methylated, preferably a 2-O-methylated adenosine
ccgagccgnuuguacc

SEQ ID NO: 140 - RNA, 16 nt, Artificial Sequence
  modified ORN 7
  modified_base (10)..(10): n can be any nucleotide, wherein the nucleotide is 2-O-methylated, preferably a 2-O-methylated uracil
ccgagccganuguacc

SEQ ID NO: 141 - RNA, 16 nt, Artificial Sequence
  modified ORN 8
  modified_base (11)..(11): n can be any nucleotide, wherein the nucleotide is 2-O-methylated, preferably a 2-O-methylated uracil
ccgagccgaunguacc

SEQ ID NO: 142 - RNA, 16 nt, Artificial Sequence
  modified ORN 9
  modified_base (12)..(12): n can be any nucleotide, wherein the nucleotide is 2-O-methylated, preferably a 2-O-methylated guanosine
ccgagccgauunuacc

SEQ ID NO: 143 - RNA, 16 nt, Artificial Sequence
  modified ORN 10
  modified_base (8)..(8): n can be any nucleotide, wherein the nucleotide is 2-O-methylated, preferably a 2-O-methylated guanosine
ccgagccnauuguacc

SEQ ID NO: 144 - RNA, 16 nt, Artificial Sequence
  modified ORN 11
  modified_base (15)..(15): n can be any nucleotide, wherein the nucleotide is 2-O-methylated, preferably a 2-O-methylated cytosine
ccgagccgauuguanc

SEQ ID NO: 145 - RNA, 16 nt, Artificial Sequence
  modified ORN 12
  modified_base (15)..(15): n can be any nucleotide, wherein the nucleotide is 2-O-methylated, preferably a 2-O-methylated cytosine
ccgagccgauuguanc

SEQ ID NO: 146 - RNA, 16 nt, Artificial Sequence
  modified ORN 13
  modified_base (7)..(7): n can be any nucleotide, wherein the nucleotide is 2-O-methylated, preferably a 2-O-methylated cytosine
ccgagcngauuguacc

SEQ ID NO: 147 - RNA, 16 nt, Artificial Sequence
  modified ORN 14
  modified_base (8)..(8): n can be any nucleotide, wherein the nucleotide is 2-O-methylated, preferably a 2-O-methylated guanosine
ccgagccncuuguccc

SEQ ID NO: 148 - RNA, 16 nt, Artificial Sequence
  modified ORN 15
  modified_base (13)..(13): n can be any nucleotide, wherein the nucleotide is 2-O-methylated, preferably a 2-O-methylated uracil
ccgagccgcuugnccc

SEQ ID NO: 149 - RNA, 9 nt, Artificial Sequence
  9mer oligonucleotide Gm
  modified_base (5)..(5): n can be any nucleotide, wherein the nucleotide is 2-O-methylated, preferably a 2-O-methylated guanosine
gagangcca

SEQ ID NO: 150 - RNA, 9 nt, Artificial Sequence
  9mer oligonucleotide Gm
  modified_base (5)..(5): n can be any nucleotide, wherein the nucleotide is 2-O-methylated, preferably a 2-O-methylated guanosine
gaggngcca

SEQ ID NO: 151 - RNA, 9 nt, Artificial Sequence
  9mer oligonucleotide Am
  modified_base (5)..(5): n can be any nucleotide, wherein the nucleotide is 2-O-methylated, preferably a 2-O-methylated adenosine
gagungcca

SEQ ID NO: 152 - RNA, 9 nt, Artificial Sequence
  9mer oligonucleotide Cm
  modified_base (5)..(5): n can be any nucleotide, wherein the nucleotide is 2-O-methylated, preferably a 2-O-methylated cytosine
gagcngcca

SEQ ID NO: 153 - RNA, 9 nt, Artificial Sequence
  9mer oligonucleotide Um
  modified_base (5)..(5): n can be any nucleotide, wherein the nucleotide is 2-O-methylated, preferably a 2-O-methylated uracil
gagcngcca

SEQ ID NO: 154 - RNA, 9 nt, Artificial Sequence
  9mer oligonucleotide Gm
  modified_base (5)..(5): n can be any nucleotide, wherein the nucleotide is 2-O-methylated, preferably a 2-O-methylated guanosine
gagcnacca

SEQ ID NO: 155 - RNA, 9 nt, Artificial Sequence
  9mer oligonucleotide Gm
  modified_base (5)..(5): n can be any nucleotide, wherein the nucleotide is 2-O-methylated, preferably a 2-O-methylated guanosine
gagcnccca

SEQ ID NO: 156 - RNA, 9 nt, Artificial Sequence
  9mer oligonucleotide Gm
  modified_base (5)..(5): n can be any nucleotide, wherein the nucleotide is 2-O-methylated, preferably a 2-O-methylated guanosine
gagcnucca

SEQ ID NO: 157 - RNA, 9 nt, Artificial Sequence
  9mer oligonucleotide Gm
  modified_base (5)..(5): n can be any nucleotide, wherein the nucleotide is 2-O-methylated, preferably a 2-O-methylated guanosine
gagcngaca

SEQ ID NO: 158 - RNA, 9 nt, Artificial Sequence
  9mer oligonucleotide Gm
  modified_base (5)..(5): n can be any nucleotide, wherein the nucleotide is 2-O-methylated, preferably a 2-O-methylated guanosine
gagcnggca

SEQ ID NO: 159 - RNA, 9 nt, Artificial Sequence
  9mer oligonucleotide Gm
  modified_base (5)..(5): n can be any nucleotide, wherein the nucleotide is 2-O-methylated, preferably a 2-O-methylated guanosine
gagcnguca

SEQ ID NO: 160 - RNA, 9 nt, Artificial Sequence
  9mer oligonucleotide Gm
  modified_base (6)..(6): n can be any nucleotide, wherein the nucleotide is 2-O-methylated, preferably a 2-O-methylated guanosine
gagcgncca

SEQ ID NO: 161 - RNA, 9 nt, Artificial Sequence
  9mer oligonucleotide Gm
  modified_base (7)..(7): n can be any nucleotide, wherein the nucleotide is 2-O-methylated, preferably a 2-O-methylated guanosine
gagcggnca

SEQ ID NO: 162 - RNA, 9 nt, Artificial Sequence
  9mer oligonucleotide Gm
  modified_base (8)..(8): n can be any nucleotide, wherein the nucleotide is 2-O-methylated, preferably a 2-O-methylated guanosine
gagcggcna

SEQ ID NO: 163 - RNA, 9 nt, Artificial Sequence
  9mer oligonucleotide Gm
  modified_base (4)..(4): n can be any nucleotide, wherein the nucleotide is 2-O-methylated, preferably a 2-O-methylated guanosine
gagnggcca

SEQ ID NO: 164 - RNA, 9 nt, Artificial Sequence
  9mer oligonucleotide Gm
  modified_base (3)..(3): n can be any nucleotide, wherein the nucleotide is 2-O-methylated, preferably a 2-O-methylated guanosine
gancggcca

SEQ ID NO: 165 - RNA, 9 nt, Artificial Sequence
  9mer oligonucleotide Gm
  modified_base (2)..(2): n can be any nucleotide, wherein the nucleotide is 2-O-methylated, preferably a 2-O-methylated guanosine
gngcggcca

SEQ ID NO: 166 - DNA, 18 bp, Artificial Sequence
  IRS-954 (DV-1079)
tgctcctggaggggttgt

SEQ ID NO: 167 - DNA, 18 bp, Artificial Sequence
  IRO-5
ctatctgacgttctctgt

SEQ ID NO: 168 - DNA, 15 bp, Artificial Sequence
  IRS 2088
tcctggcggggaagt

SEQ ID NO: 169 - DNA, 15 bp, Artificial Sequence
  IRS 869
tcctggaggggttgt

SEQ ID NO: 170 - DNA, 15 bp, Artificial Sequence
  INH-ODN-2114
tcctggaggggaagt

SEQ ID NO: 171 - DNA, 15 bp, Artificial Sequence
  INH-ODN 4024
tcctggatgggaagt

SEQ ID NO: 172 - DNA, 12 bp, Artificial Sequence
  INH-ODN 4084-F
cctggatgggaa

SEQ ID NO: 173 - DNA, 20 bp, Artificial Sequence
  IRS-661
tgcttgcaagcttgcaagca

SEQ ID NO: 174 - DNA, 18 bp, Artificial Sequence
  IRS-954
tgctcctggaggggttgt

SEQ ID NO: 175 - DNA, 15 bp, Artificial Sequence
  INH-ODN-24888
tcctggcggggaagt

SEQ ID NO: 176 - DNA, 15 bp, Artificial Sequence
  IHN-ODN 2088
tcctggcggggaagt

SEQ ID NO: 177 - DNA, 24 bp, Artificial Sequence
  ODN A151
ttagggttagggttagggttaggg

SEQ ID NO: 178 - DNA, 21 bp, Artificial Sequence
  G-ODN
ctcctattgggggtttcctat

SEQ ID NO: 179 - DNA, 24 bp, Artificial Sequence
  ODN INH-1
cctggatgggaattcccatccagg

SEQ ID NO: 180 - DNA, 24 bp, Artificial Sequence
  ODN INH-18
cctggatgggaacttaccgctgca

SEQ ID NO: 181 - DNA, 24 bp, Artificial Sequence
  INH-4
ttcccatccaggcctggatgggaa

SEQ ID NO: 182 - DNA, 24 bp, Artificial Sequence
  INH-13
cttaccgctgcacctggatgggaa

SEQ ID NO: 183 - DNA, 24 bp, Artificial Sequence
  (pS-) ST-ODN
tcgtcgttttgtcgttttgtcgtt

SEQ ID NO: 184 - DNA, 15 bp, Artificial Sequence
  INH-ODN 21 14
tcctggaggggaagt

SEQ ID NO: 185 - DNA, 133 bp, Artificial Sequence
  Ndufa1(G51C) 3′-UTR
ggaagcattttcctggctgattaaaagaaattactcagctatggtcatctcttcctgttagaaggctatgcagcatattatatactatgcgcatgttatgaaatgcataataaaaaattttaaaaaatctaaa

SEQ ID NO: 186 - RNA, 133 nt, Artificial Sequence
  Ndufa1(G51C) 3′-UTR
ggaagcauuuuccuggcugauuaaaagaaauuacucagcuauggucaucucuuccuguuagaaggcuaugcagcauauuauauacuaugcgcauguuaugaaaugcauaauaaaaaauuuuaaaaaaucuaaa

SEQ ID NO: 187 - RNA, 9 nt, Artificial Sequence
  9mer oligonucleotide Gm PTO (Gm18 variant 1)
  modified_base (1)..(9): wherein all internucleotidelinkages are phosphothioate linkages
  modified_base (5)..(5): n can be any nucleotide, wherein the nucleotide is 2-O-methylated, preferably a 2-O-methylated guanosine
gagcngcca

SEQ ID NO: 188 - RNA, 9 nt, Artificial Sequence
  9mer oligonucleotide Gm (Gm18 variant 3)
  modified_base (3)..(3): n can be any nucleotide, wherein the nucleotide is 2-O-methylated, preferably a 2-O-methylated guanosine
gcngccaaa

SEQ ID NO: 189 - RNA, 9 nt, Artificial Sequence
  9mer oligonucleotide Gm PTO (Gm18 variant 4)
  modified_base (1)..(9): wherein all internucleotidelinkages are phosphothioate linkages
  modified_base (3)..(3): n can be any nucleotide, wherein the nucleotide is 2-O-methylated, preferably a 2-O-methylated guanosine
gcngccaaa

SEQ ID NO: 190 - RNA, 9 nt, Artificial Sequence
  9mer oligonucleotide Gm
  modified_base (7)..(7): n can be any nucleotide, wherein the nucleotide is 2-O-methylated, preferably a 2-O-methylated guanosine
ccgagcngc

SEQ ID NO: 191 - RNA, 9 nt, Artificial Sequence
  9mer oligonucleotide Gm, m6a
  modified_base (2)..(2): wherein n is N6-methyladenosine
  modified_base (5)..(5): n can be any nucleotide, wherein the nucleotide is 2-O-methylated, preferably a 2-O-methylated guanosine
  modified_base (9)..(9): wherein n is N6-methyladenosine
gngcngccn

SEQ ID NO: 192 - RNA, 12 nt, Artificial Sequence
  12mer oligonucleotide Gm, ac4c
  modified_base (5)..(5): wherein n is 4-acetylcytidine
  modified_base (6)..(6): n can be any nucleotide, wherein the nucleotide is 2-O-methylated, preferably a 2-O-methylated guanosine
  modified_base (9)..(9): wherein n is 4-acetylcytidine
  modified_base (11)..(11): wherein n is 4-acetylcytidine
gagcnngcncna

SEQ ID NO: 193 - DNA, 15 bp, Artificial Sequence
  ODN 2088 PTO (DNA oligo 1)
  modified_base (1)..(15): wherein all internucleotidelinkages are phosphothioate linkages
tcctggcggggaagt

SEQ ID NO: 194 - DNA, 15 bp, Artificial Sequence
  ODN 2088 control (ODN 20959) PTO (DNA oligo 2)
  modified_base (1)..(15): wherein all internucleotidelinkages are phosphothioate linkages
taatggcggggaagt

SEQ ID NO: 195 - DNA, 15 bp, Artificial Sequence
  ODN 2088 control (ODN 2087) PTO
  modified_base (1)..(15): wherein all internucleotidelinkages are phosphothioate linkages
tcctgagcttgaagt

SEQ ID NO: 196 - DNA, 15 bp, Artificial Sequence
  ODN 2088 control (ODN 20958) PTO
  modified_base (1)..(15): wherein all internucleotidelinkages are phosphothioate linkages
tcctaacaaaaaaat

SEQ ID NO: 197 - DNA, 18 bp, Artificial Sequence
  IRS-954 (DV-1079) PTO
  modified_base (1)..(18): wherein all internucleotidelinkages are phosphothioate linkages
tgctcctggaggggttgt

SEQ ID NO: 198 - DNA, 20 bp, Artificial Sequence
  IRS-661 PTO
  modified_base (1)..(20): wherein all internucleotidelinkages are phosphothioate linkages
tgcttgcaagcttgcaagca

SEQ ID NO: 199 - DNA, 15 bp, Artificial Sequence
  INH-ODN-24888 PTO Em PTO
  modified_base (1)..(15): wherein all internucleotidelinkages are phosphothioate linkages
  modified_base (8)..(8): wherein n is 7-deaza-2′-O-methyl-guanine
tcctggcngggaagt

SEQ ID NO: 200 - DNA, 18 bp, Artificial Sequence
  IRO-5
  modified_base (7)..(7): n can be any nucleotide, wherein the nucleotide is 2-O-methylated, preferably a 2-O-methylated guanosine
  modified_base (8)..(8): n can be any nucleotide, wherein the nucleotide is 2-O-methylated, preferably a 2-O-methylated adenosine
ctatctnncgttctctgt

SEQ ID NO: 201 - RNA, 17 nt, Artificial Sequence
  SM-MePS PTO (RNA oligo 1)
  modified_base (1)..(17): wherein all internucleotidelinkages are phosphothioate linkages
  modified_base (1)..(1): n can be any nucleotide, wherein the nucleotide is 2-O-methylated, preferably a 2-O-methylated adenosine
  modified_base (2)..(2): n can be any nucleotide, wherein the nucleotide is 2-O-methylated, preferably a 2-O-methylated uracil
  modified_base (4)..(4): n can be any nucleotide, wherein the nucleotide is 2-O-methylated, preferably a 2-O-methylated adenosine
  modified_base (5)..(5): n can be any nucleotide, wherein the nucleotide is 2-O-methylated, preferably a 2-O-methylated uracil
  modified_base (9)..(10): n can be any nucleotide, wherein the nucleotide is 2-O-methylated, preferably a 2-O-methylated uracil
  modified_base (14)..(14): n can be any nucleotide, wherein the nucleotide is 2-O-methylated, preferably a 2-O-methylated adenosine
  modified_base (15)..(15): n can be any nucleotide, wherein the nucleotide is 2-O-methylated, preferably a 2-O-methylated uracil
nnannuuunnggunnuu

SEQ ID NO: 202 - RNA, 12 nt, Artificial Sequence
  12mer oligonucleotide Um PTO
  modified_base (1)..(12): wherein all internucleotidelinkages are phosphothioate linkages
  modified_base (3)..(3): n can be any nucleotide, wherein the nucleotide is 2-O-methylated, preferably a 2-O-methylated uracil
ganacuuaccug

SEQ ID NO: 203 - RNA, 18 nt, Artificial Sequence
  18mer oligonucleotide Um, Gm, Cm, Am (RNA oligo 5)
  modified_base (1)..(1): n can be any nucleotide, wherein the nucleotide is 2-O-methylated, preferably a 2-O-methylated uracil
  modified_base (2)..(2): n can be any nucleotide, wherein the nucleotide is 2-O-methylated, preferably a 2-O-methylated guanosine
  modified_base (3)..(3): n can be any nucleotide, wherein the nucleotide is 2-O-methylated, preferably a 2-O-methylated cytosine
  modified_base (4)..(4): n can be any nucleotide, wherein the nucleotide is 2-O-methylated, preferably a 2-O-methylated uracil
  modified_base (5)..(6): n can be any nucleotide, wherein the nucleotide is 2-O-methylated, preferably a 2-O-methylated cytosine
  modified_base (7)..(7): n can be any nucleotide, wherein the nucleotide is 2-O-methylated, preferably a 2-O-methylated uracil
  modified_base (8)..(9): n can be any nucleotide, wherein the nucleotide is 2-O-methylated, preferably a 2-O-methylated guanosine
  modified_base (10)..(10): n can be any nucleotide, wherein the nucleotide is 2-O-methylated, preferably a 2-O-methylated adenosine
  modified_base (11)..(14): n can be any nucleotide, wherein the nucleotide is 2-O-methylated, preferably a 2-O-methylated guanosine
  modified_base (15)..(16): n can be any nucleotide, wherein the nucleotide is 2-O-methylated, preferably a 2-O-methylated uracil
  modified_base (17)..(17): n can be any nucleotide, wherein the nucleotide is 2-O-methylated, preferably a 2-O-methylated guanosine
nnnnnnnnnnnnnnnnnu

SEQ ID NO: 204 - RNA, 21 nt, Artificial Sequence
  2OMe 2 xG(S) (RNA oligo 3)
  modified_base (6)..(6): n can be any nucleotide, wherein the nucleotide is 2-O-methylated, preferably a 2-O-methylated guanosine
  modified_base (8)..(8): n can be any nucleotide, wherein the nucleotide is 2-O-methylated, preferably a 2-O-methylated guanosine
uugaununuuuagucgcuauu

SEQ ID NO: 205 - RNA, 27 nt, Artificial Sequence
  27mer oligonucleotide Gm (RNA oligo 4)
  modified_base (17)..(17): n can be any nucleotide, wherein the nucleotide is 2-O-methylated, preferably a 2-O-methylated guanosine
ggugggguucccgagcngccaaaggga

SEQ ID NO: 206 - RNA, 10 nt, Artificial Sequence
  10-mer (Um)
  modified_base (3)..(3): n can be any nucleotide, wherein the nucleotide is 2-O-methylated, preferably a 2-O-methylated uracil
  modified_base (5)..(5): n can be any nucleotide, wherein the nucleotide is 2-O-methylated, preferably a 2-O-methylated uracil
  modified_base (8)..(10): n can be any nucleotide, wherein the nucleotide is 2-O-methylated, preferably a 2-O-methylated uracil
ggncnacnnn

SEQ ID NO: 207 - RNA, 21 nt, Artificial Sequence
  (mU)21
  modified_base (1)..(21): n can be any nucleotide, wherein the nucleotide is 2-O-methylated, preferably a 2-O-methylated uracil
nnnnnnnnnnnnnnnnnnnnn

SEQ ID NO: 208 - RNA, 15 nt, Artificial Sequence
  (mU)15
  modified_base (1)..(15): n can be any nucleotide, wherein the nucleotide is 2-O-methylated, preferably a 2-O-methylated uracil
nnnnnnnnnnnnnnn

SEQ ID NO: 209 - RNA, 10 nt, Artificial Sequence
  (mU)10
  modified_base (1)..(10): n can be any nucleotide, wherein the nucleotide is 2-O-methylated, preferably a 2-O-methylated uracil
nnnnnnnnnn

SEQ ID NO: 210 - RNA, 21 nt, Artificial Sequence
  gal-mU
  modified_base (2)..(2): n can be any nucleotide, wherein the nucleotide is 2-O-methylated, preferably a 2-O-methylated uracil
  modified_base (10)..(10): n can be any nucleotide, wherein the nucleotide is 2-O-methylated, preferably a 2-O-methylated uracil
  modified_base (17)..(19): n can be any nucleotide, wherein the nucleotide is 2-O-methylated, preferably a 2-O-methylated uracil
cnacacaaancagcgannnuu

SEQ ID NO: 211 - RNA, 21 nt, Artificial Sequence
  gal-mC
  modified_base (1)..(1): n can be any nucleotide, wherein the nucleotide is 2-O-methylated, preferably a 2-O-methylated cytosine
  modified_base (4)..(4): n can be any nucleotide, wherein the nucleotide is 2-O-methylated, preferably a 2-O-methylated cytosine
  modified_base (6)..(6): n can be any nucleotide, wherein the nucleotide is 2-O-methylated, preferably a 2-O-methylated cytosine
  modified_base (11)..(11): n can be any nucleotide, wherein the nucleotide is 2-O-methylated, preferably a 2-O-methylated cytosine
  modified_base (14)..(14): n can be any nucleotide, wherein the nucleotide is 2-O-methylated, preferably a 2-O-methylated cytosine
nuananaaaunagngauuuuu

SEQ ID NO: 212 - RNA, 21 nt, Artificial Sequence
  gal-mA
  modified_base (3)..(3): n can be any nucleotide, wherein the nucleotide is 2-O-methylated, preferably a 2-O-methylated adenosine
  modified_base (5)..(5): n can be any nucleotide, wherein the nucleotide is 2-O-methylated, preferably a 2-O-methylated adenosine
  modified_base (7)..(9): n can be any nucleotide, wherein the nucleotide is 2-O-methylated, preferably a 2-O-methylated adenosine
  modified_base (12)..(12): n can be any nucleotide, wherein the nucleotide is 2-O-methylated, preferably a 2-O-methylated adenosine
  modified_base (16)..(16): n can be any nucleotide, wherein the nucleotide is 2-O-methylated, preferably a 2-O-methylated adenosine
cuncncnnnucngcgnuuuuu